(19) United States
(12) Patent Application Publication
(10) Pub. No.: US 2024/0173539 A1
(43) Pub. Date: May 30, 2024

(54) INTRAVASCULAR BLOOD PUMP

(71) Applicant: PROCYRION, INC., Houston, TX (US)

(72) Inventors: Ronald G. EARLES, Houston, TX (US); Omar M. BENAVIDES, Houston, TX (US); Chistopher A. DURST, Houston, TX (US); Jason J. HEURING, Houston, TX (US)

(21) Appl. No.: 18/525,169

(22) Filed: Nov. 30, 2023

Related U.S. Application Data
(63) Continuation of application No. PCT/US2022/031627, filed on May 31, 2022.
(60) Provisional application No. 63/196,078, filed on Jun. 2, 2021.

(51) Int. Cl. A61M 60/104; A61M 60/216; A61M 60/408; A61M 60/422; A61M 60/818

(52) U.S. Cl. CPC A61M 60/104; A61M 60/216; A61M 60/408; A61M 60/422; A61M 60/818

(57) ABSTRACT

A blood flow assist system can include a blood pump and an elongate power lead having a distal end portion connected to the blood pump and a proximal end portion opposite the distal end portion. The power lead can include a lumen extending distally from the proximal end portion of the power lead along a longitudinal axis of the blood flow assist system and one or more electrical contacts disposed on an outer surface of the lead at the proximal end portion of the elongate power lead. The power lead can include a recess extending into the proximal portion of the power lead in a direction transverse to the longitudinal axis, the recess configured to receive a locking pin to releasable connect the power lead to an external device.

### CROSS-REFERENCE TO RELATED APPLICATIONS

This application claims priority to U.S. Provisional Patent Application No. 63/196,078, filed Jun. 2, 2021, the entire contents of which are incorporated by reference herein in their entirety and for all purposes.

### BACKGROUND

### Field

The field relates to percutaneous medical devices and, in particular, to intravascular device such as intravascular blood pumps.

### Description of the Related Art

In the field of cardiac assist devices and mechanical circulatory support, implanted blood pumps are placed in direct communication with a heart chamber or in a blood vessel (e.g., in an aorta) and are then used to support the heart in pumping blood out of the heart chamber or in moving blood through the blood vessel (e.g. to enhance perfusion to the kidneys or other organs). Some blood pumps are intravascular blood pumps and are designed or adapted to draw in or discharge blood within blood vessels.

### SUMMARY

In various embodiments, a blood flow assist system is disclosed. The blood flow assist system can include a blood pump and an elongate power lead having a distal end portion connected to the blood pump and a proximal end portion opposite the distal end portion. The power lead can include a lumen extending distally from the proximal end portion of the power lead along a longitudinal axis of the blood flow assist system.

In some embodiments, one or more electrical contacts are disposed on an outer surface of the lead at the proximal end portion of the elongate power lead. In some embodiments, a recess extends into the proximal portion of the power lead in a direction transverse to the longitudinal axis, the recess configured to receive a locking pin to releasably connect the power lead to an external device.

In some embodiments, the lumen is an inner lumen and further comprising a plurality of outer lumens disposed around the inner lumen, the plurality of outer lumens extending along the longitudinal axis. In some embodiments, the system can include a plurality of elongate conductors, each elongate conductor of the plurality of elongate conductors extending through a corresponding outer lumen of the plurality of outer lumens. In some embodiments, the one or more electrical contacts comprises a plurality of electrical contacts spaced apart along the longitudinal axis on the outer surface of the lead, wherein each elongate conductor of the plurality of elongate conductors is electrically connected to a corresponding electrical contact of the plurality of electrical contacts. In some embodiments, each electrical contact of the plurality of electrical contacts comprises a ring, wherein adjacent electrical contacts are spaced apart by an insulating material. In some embodiments, the elongate lead comprises an insulating material along an outer surface thereof, the insulating material comprising polyurethane. In some embodiments, the one or more electrical contacts are disposed distal the recess. In some embodiments, the recess is disposed distal a proximal end of the elongate power lead. In some embodiments, a thickness of the elongate power lead is not uniform along a length of the elongate power lead. In some embodiments, the blood flow assist system can include a delivery system to deliver the blood pump to a target location in a patient. The delivery system can include a proximal handle having a lumen therethrough, the lumen sized and shaped to receive the proximal end portion of the elongate power lead, the proximal handle comprising a lead retention device connectable to the elongate power lead, the proximal handle further comprising a lead release assembly configured to release the elongate power lead from the lead retention device; a delivery catheter extending distally from the proximal handle; a transfer stop disposed about the delivery catheter distal the proximal handle, the transfer stop comprising a transfer stop lock having a locked configuration and an unlocked configuration, the delivery catheter slidable relative to the transfer stop in the unlocked configuration and slidably locked relative to the transfer stop in the locked configuration; and a distal handle disposed about the delivery catheter distal the transfer stop, the distal handle comprising a handle lock having a locked configuration and an unlocked configuration, the delivery catheter slidable relative to the distal handle in the unlocked configuration and slidably locked relative to the distal handle in the locked configuration, the distal handle comprising a cavity configured to house the at least a distal portion of the blood pump. In some embodiments, the blood flow assist system can include a retrieval system configured to remove the blood pump from a patient. The retrieval system can include a retrieval dilator having a retrieval dilator hub and a clamping member at a proximal portion of the retrieval dilator, the retrieval dilator having a lumen sized and shaped to receive the elongate power lead therethrough, the clamping member having a clamped configuration in which the clamping member clamps against the elongate power lead and an unclamped configuration in which the elongate power lead is slidable relative to the clamping member; a retrieval sheath having a retrieval sheath hub at a proximal portion of the retrieval sheath, the retrieval sheath having a lumen sized and shaped to receive the retrieval dilator therethrough; and a retrieval handle having a lumen sized and shaped to receive the retrieval dilator therethrough such that the retrieval dilator extends through the retrieval handle and the retrieval sheath during a retrieval procedure, the retrieval handle having a distal connector configured to connect to the retrieval sheath hub and a proximal connector configured to connect to the retrieval dilator hub.

In another embodiment, a percutaneous medical system is disclosed. The percutaneous medical system can include a medical device and an elongate lead having a distal end portion connected to the medical device and a proximal end portion opposite the distal end portion. The lead can include a lumen extending distally from the proximal end portion of the lead along a longitudinal axis of the system. The lead can include a recess extending into the proximal end portion of the lead in a direction transverse to the longitudinal axis, the recess configured to receive a locking pin to releasable connect the lead to an external device.

In some embodiments, the elongate lead comprises an elongate power lead configured to convey current to the medical device. In some embodiments, the system can include one or more electrical contacts disposed on an outer surface of the lead at the proximal end portion of the elongate power lead. In some embodiments, the system can include a plurality of outer lumens disposed around the lumen, the plurality of outer lumens extending along the longitudinal axis. In some embodiments, the system can include a plurality of elongate conductors, each elongate conductor of the plurality of elongate conductors extending through a corresponding outer lumen of the plurality of outer lumens. In some embodiments, each elongate conductor of the plurality of elongate conductors is electrically connected to a corresponding electrical contact on exposed on an outer surface of the lead. In some embodiments, each electrical contact comprises a ring, wherein adjacent electrical contacts are spaced apart by an insulating material. In some embodiments, the elongate lead comprises an insulating material along an outer surface thereof, the insulating material comprising polyurethane. In some embodiments, a thickness of the elongate lead is not uniform along a length of the elongate lead.

In another embodiment, a blood flow assist system is disclosed. The blood flow assist system can include a blood pump and an elongate power lead having a distal end portion connected to the blood pump and a proximal end portion opposite the distal end portion, the power lead comprising one or more elongate conductors extending through the elongate power lead along a longitudinal axis of the blood flow assist system, the one or more elongate conductors connected to the blood pump to convey current to the blood pump. The system can include a handle disposed proximal the blood pump, the handle configured to connect to the elongate power lead.

In some embodiments, the elongate power lead comprises one or more electrical contacts electrically connected to a corresponding elongate conductor of the one or more elongate conductors. In some embodiments, the handle is releasably connectable to the elongate power lead, and wherein the one or more electrical contacts are configured to connect to an external control system following release of the elongate power lead from the handle. In some embodiments, the handle comprises an electrical port configured to electrically connect to the one or more electrical contacts with the handle connected to the elongate power lead. In some embodiments, the system can include one or more lumens extending distally from the proximal end portion of the power lead along the longitudinal axis, the one or more elongate conductors extending through a corresponding lumen of the one or more lumens. In some embodiments, the one or more lumens comprises a central lumen and a plurality of outer lumens disposed about the central lumen, the one or more elongate conductors comprising a plurality of elongate conductors, each of the one or more elongate conductors extending through a corresponding outer lumen of the plurality of outer lumens. In some embodiments, the one or more electrical contacts are disposed on an outer surface of the lead at the proximal end portion of the elongate power lead. In some embodiments, each of the one or more electrical contacts comprises a ring. In some embodiments, the system can include the elongate power lead comprises a recess extending into the proximal portion of the power lead in a direction transverse to the longitudinal axis, the recess configured to receive a locking pin of the handle to releasable connect the power lead to the handle. In some embodiments, the elongate power lead comprises an insulating material along an outer surface thereof, the insulating material comprising polyurethane. In some embodiments, a thickness of the elongate power lead is not uniform along a length of the elongate power lead.

In another embodiment, an elongate power lead can include a distal end portion configured to connect to a blood pump and a proximal end portion opposite the distal end portion; a lumen extending distally from the proximal end portion of the power lead along a longitudinal axis; one or more electrical contacts disposed on an outer surface of the lead at the proximal end portion of the elongate power lead; and a recess extending into the proximal portion of the power lead in a direction transverse to the longitudinal axis, the recess configured to receive a locking pin to releasably connect the power lead to an external device.

In some embodiments, the lumen is an inner lumen and further comprising a plurality of outer lumens disposed around the inner lumen, the plurality of outer lumens extending along the longitudinal axis. In some embodiments, the elongate power lead can include a plurality of elongate conductors, each elongate conductor of the plurality of elongate conductors extending through a corresponding outer lumen of the plurality of outer lumens. In some embodiments, the one or more electrical contacts comprises a plurality of electrical contacts spaced apart along the longitudinal axis on the outer surface of the lead, wherein each elongate conductor of the plurality of elongate conductors is electrically connected to a corresponding electrical contact of the plurality of electrical contacts. In some embodiments, each electrical contact of the plurality of electrical contacts comprises a ring, wherein adjacent electrical contacts are spaced apart by an insulating material. In some embodiments, the elongate lead comprises an insulating material along an outer surface thereof, the insulating material comprising polyurethane. In some embodiments, the one or more electrical contacts are disposed distal the recess. In some embodiments, the recess is disposed distal a proximal end of the elongate power lead. In some embodiments, a thickness of the elongate power lead is not uniform along a length of the elongate power lead.

In another embodiment, an elongate lead can include: a distal end portion configured to connect to a medical device and a proximal end portion opposite the distal end portion; a lumen extending distally from the proximal end portion of the lead along a longitudinal axis; and a recess extending into the proximal end portion of the lead in a direction transverse to the longitudinal axis, the recess configured to receive a locking pin to releasable connect the lead to an external device.

In some embodiments, the elongate lead comprises an elongate power lead configured to convey current to the medical device. In some embodiments, the elongate power lead can include one or more electrical contacts disposed on an outer surface of the lead at the proximal end portion of the elongate power lead. In some embodiments, the elongate power lead can include a plurality of outer lumens disposed around the lumen, the plurality of outer lumens extending along the longitudinal axis. In some embodiments, the elongate power lead can include a plurality of elongate conductors, each elongate conductor of the plurality of elongate conductors extending through a corresponding outer lumen of the plurality of outer lumens. In some embodiments, each elongate conductor of the plurality of elongate conductors is electrically connected to a corresponding electrical contact on exposed on an outer surface of the lead. In some embodiments, each electrical contact comprises a ring, wherein adjacent electrical contacts are spaced apart by an insulating material. In some embodiments, the elongate lead comprises an insulating material along an outer surface thereof, the insulating material comprising polyurethane. In some embodiments, a thickness of the elongate lead is not uniform along a length of the elongate lead.

In another embodiment, a delivery system for an intravascular blood pump is disclosed. The delivery system can include a proximal handle having a lumen therethrough, the lumen sized and shaped to receive a proximal end portion of a power lead connected to the blood pump, the proximal handle comprising a lead retention device connectable to the power lead, the proximal handle further comprising a lead release assembly configured to release the power lead from the lead retention device; a delivery catheter extending distally from the proximal handle; a transfer stop disposed about the delivery catheter distal the proximal handle, the transfer stop comprising a transfer stop lock having a locked configuration and an unlocked configuration, the delivery catheter slidable relative to the transfer stop in the unlocked configuration and slidably locked relative to the transfer stop in the locked configuration; and a distal handle disposed about the delivery catheter distal the transfer stop, the distal handle comprising a handle lock having a locked configuration and an unlocked configuration, the delivery catheter slidable relative to the distal handle in the unlocked configuration and slidably locked relative to the distal handle in the locked configuration, the distal handle comprising a cavity configured to house the at least a distal portion of an intravascular blood pump.

In some embodiments, the proximal handle comprises an electrical port configured to electrically connect the power lead to an external control system. In some embodiments, the proximal handle comprises a fluid port configured to deliver fluid to the lumen of the proximal handle.

In some embodiments, an intravascular blood pump can comprise the delivery system. The intravascular blood pump can include a delivery sheath having a delivery sheath hub connectable to a distal connector of the distal handle, the delivery sheath comprising a lumen in communication with a distal hypotube of the distal handle when the delivery sheath is connected to the distal handle. In some embodiments, at least a distal portion of the blood pump is disposed in a cavity of the distal handle, the inner catheter slidable through the distal handle to push the blood pump through a vasculature of the patient. In some embodiments, the blood pump comprises an impeller at least partially disposed in a shroud and a plurality of self-expanding struts extending from the shroud.

In another embodiment, a delivery system for a percutaneous blood pump is disclosed. The delivery system can include a proximal handle connected to an elongate lead of the intravascular blood pump; a delivery catheter extending distally from the proximal handle, the lead extending through a lumen of the delivery catheter; and a distal handle disposed about the delivery catheter distal the proximal handle, the distal handle comprising a cavity in which at least a distal portion of the percutaneous blood pump is disposed, the lead connected to a proximal portion of the intravascular blood pump.

In some embodiments, the distal handle comprises a handle lock having a locked configuration and an unlocked configuration, the delivery catheter slidable relative to the distal handle in the unlocked configuration and slidably locked relative to the distal handle in the locked configuration. In some embodiments, the system can include a transfer stop disposed about the delivery catheter between the distal handle and the proximal handle, the transfer stop comprising a transfer stop lock having a locked configuration and an unlocked configuration, the delivery catheter slidable relative to the transfer stop in the unlocked configuration and slidably locked relative to the transfer stop in the locked configuration. In some embodiments, the lead comprises a power lead configured to convey current to the blood pump, the proximal handle comprising an electrical port configured to electrically connect the power lead to an external control system. In some embodiments, the proximal handle comprises a fluid port configured to deliver fluid to a lumen of the proximal handle. In some embodiments, the system can include a metallic cap on a distal end of the delivery catheter.

In some embodiments, a percutaneous blood pump comprises the delivery system. The percutaneous blood pump can include a delivery sheath having a delivery sheath hub connectable to a distal connector of the distal handle, the delivery sheath comprising a lumen in communication with a distal hypotube of the distal handle when the delivery sheath is connected to the distal handle. In some embodiments, the blood pump comprises an impeller at least partially disposed in a shroud and a plurality of self-expanding struts extending from the shroud.

In another embodiment, a delivery system for a percutanous medical device is disclosed. The delivery system can include a delivery catheter; a transfer stop disposed about the delivery catheter, the transfer stop comprising a transfer stop lock having a locked configuration and an unlocked configuration, the delivery catheter slidable relative to the transfer stop in the unlocked configuration and slidably locked relative to the transfer stop in the locked configuration; and a distal handle disposed about the delivery catheter distal the transfer stop, the distal handle comprising a handle lock having a locked configuration and an unlocked configuration, the delivery catheter slidable relative to the distal handle in the unlocked configuration and slidably locked relative to the distal handle in the locked configuration.

In some embodiments, the system can include a proximal handle disposed proximal the distal handle and the transfer stop, the delivery catheter extending distally from the proximal handle. In some embodiments, the proximal handle includes a housing body having a lumen therethrough, the lumen sized and shaped to receive a proximal end portion of an elongate lead of the medical device. In some embodiments, the proximal handle comprises a lead retention device connected to the elongate lead, the proximal handle further comprising a lead release assembly configured to release the lead from the lead retention device. In some embodiments, the elongate lead comprises a power lead configured to convey current to the medical device. In some embodiments, the proximal handle comprises an electrical port configured to electrically connect the power lead to an external control system. In some embodiments, the proximal handle comprises a fluid port configured to deliver fluid to the lumen. In some embodiments, the lead retention device comprises a locking pin insertable into a recess of the lead. In some embodiments, the lead release assembly comprises a first actuator configured to remove the locking pin from the recess. In some embodiments, the lead release assembly further comprises a second actuator configured to release the lead from the housing body. In some embodiments, the system can include a metallic cap on a distal end of the delivery catheter.

In some embodiments, a percutaneous medical device can comprise the delivery system. The percutaneous medical device can include a delivery sheath having a delivery sheath hub connectable to a distal connector of the distal handle, the delivery sheath comprising a lumen in communication with a distal hypotube of the distal handle when the delivery sheath is connected to the distal handle. In some embodiments, the percutaneous medical device can include a blood pump deliverable to a target location in a body of a patient. In some embodiments, at least a distal portion of the blood pump is disposed in a cavity of the distal handle, the inner catheter slidable through the distal handle to push the blood pump through a vasculature of the patient. In some embodiments, the blood pump is connected to an elongate power lead extending proximally from the blood pump through the inner catheter. In some embodiments, the blood pump comprises an impeller at least partially disposed in a shroud and a plurality of self-expanding struts extending from the shroud.

In another embodiment, a delivery system for a percutaneous medical device is disclosed. The delivery system can include a proximal handle; a delivery catheter extending distally from the proximal handle; and a distal handle disposed about the delivery catheter distal the proximal handle, the distal handle comprising a handle lock having a locked configuration and an unlocked configuration, the delivery catheter slidable relative to the distal handle in the unlocked configuration and slidably locked relative to the distal handle in the locked configuration.

In some embodiments, the system can include a transfer stop disposed about the delivery catheter proximal the distal handle, the transfer stop comprising a transfer stop lock having a locked configuration and an unlocked configuration, the delivery catheter slidable relative to the transfer stop in the unlocked configuration and slidably locked relative to the transfer stop in the locked configuration. In some embodiments, the proximal handle includes a housing body having a lumen therethrough, the lumen sized and shaped to receive a proximal end portion of an elongate lead of the medical device. In some embodiments, the proximal handle comprises a lead retention device connected to the elongate lead, the proximal handle further comprising a lead release assembly configured to release the lead from the lead retention device. In some embodiments, the elongate lead comprises a power lead configured to convey current to the medical device. In some embodiments, the system can include an electrical port configured to electrically connect the power lead to an external control system. In some embodiments, the system can include a fluid port configured to deliver fluid to the lumen. In some embodiments, the lead retention device comprises a locking pin insertable into a recess of the lead. In some embodiments, the lead release assembly comprises a first actuator configured to remove the locking pin from the recess. In some embodiments, the lead release assembly further comprises a second actuator configured to release the lead from the housing body. In some embodiments, the system can include a metallic cap on a distal end of the delivery catheter. In some embodiments, the system can include a delivery sheath having a delivery sheath hub connectable to a distal connector of the distal handle, the delivery sheath comprising a lumen in communication with a distal hypotube of the distal handle when the delivery sheath is connected to the distal handle.

In some embodiments, a percutaneous medical device can include the delivery system. The percutaneous medical device can include a blood pump deliverable to a target location in a body of a patient. In some embodiments, at least a distal portion of the blood pump is disposed in a cavity of the distal handle, the inner catheter slidable through the distal handle to push the blood pump through a vasculature of the patient. In some embodiments, the blood pump is connected to an elongate power lead extending proximally from the blood pump through the inner catheter, the elongate power lead releasable connected to the proximal handle. In some embodiments, the blood pump comprises an impeller at least partially disposed in a shroud and a plurality of self-expanding struts extending from the shroud.

In another embodiment, a handle for a percutaneous medical device delivery system is disclosed. The handle can include a housing body having a lumen therethrough, the lumen sized and shaped to receive a proximal end portion of an elongate lead of a medical device; a lead retention device releasably connectable to the lead; and a lead release assembly configured to release the lead from the lead retention device.

In some embodiments, the elongate lead comprises a power lead configured to convey current to the medical device. In some embodiments, the handle can include an electrical port configured to electrically connect the power lead to an external control system. In some embodiments, the handle can include a fluid port configured to deliver fluid to the lumen. In some embodiments, the handle can include a delivery catheter extending distally from the housing body. In some embodiments, the lead retention device comprises a locking pin insertable into a recess of the lead. In some embodiments, the lead release assembly comprises a first actuator configured to remove the locking pin from the recess. In some embodiments, the first actuator comprises a lead unlock button slidable relative to the locking pin such that, when the lead unlock button is moved to an unlocked position, a biased spring moves the locking pin out of the recess. In some embodiments, the lead release assembly further comprises a second actuator configured to release the lead from the housing body. In some embodiments, the handle can include a plunger to which the locking pin is coupled, the plunger comprising a threaded shank extending proximally relative to the locking pin, the second actuator comprising a receiver that receives the threaded shank. In some embodiments, the second actuator comprises a tab connected to the received, the tab having a tooth that engages with the threaded shank. In some embodiments, the second actuator comprises a release knob, the receiver extending from the release knob such that rotation of the release knob pulls the threaded shank within the receiver to separate the elongate lead from the handle. In some embodiments, the handle can include a hollow shaft extending through the receiver and the threaded shank to the lead. In some embodiments, the plunger comprises at least one electrical terminal disposed distal the lead retention device. In some embodiments, the electrical terminal comprises a ring-shaped terminal sized to be disposed about a contact of the lead. In some embodiments, the handle can include an electrical board connected to the terminal by a trace, the board connected to an electrical port that provides electrical communication to an external control system. In some embodiments, the handle can include a fluid manifold distal the plunger, the fluid manifold connected to a fluid port configured to deliver fluid to the lumen of the housing body. In some embodiments, the handle can include a seal disposed in the plunger to seal against proximally-flowing fluid. In some embodiments, the handle can include one or more vent holes to provide fluid communication with an interior area to convey a sterilizing gas thereto. In some embodiments, the one or more vent holes comprises a first vent hole and a second vent hole disposed distal the first vent hole. In some embodiments, the first and second vent holes are disposed through a hollow shaft extending within the housing body, the first and second vent holes spaced apart by a distance such that, when a plunger of the proximal handle is separated from the lead, the first vent hole is covered by a portion of the plunger.

In another embodiment, a method for delivering an intravascular blood pump to a target location in a descending aorta is disclosed. The method can include inserting a delivery sheath into the descending aorta; connecting a distal handle to a delivery sheath hub at a proximal end of the delivery sheath; advancing a transfer stop distally to mate with the distal handle, the transfer stop slidably locked relative to a delivery catheter such that advancing the transfer stop slides the delivery catheter distally through the distal handle to push the blood pump out of the distal handle into a distal portion of the delivery sheath disposed within the descending aorta; unlocking the transfer stop such that the delivery catheter is slidable relative to the transfer stop; and retracting the distal handle together with the transfer stop until the transfer stop mates with a proximal handle, the retracting causing the delivery sheath to retract proximally relative to the blood pump to deploy the blood pump at the target location in the descending aorta.

In some embodiments, the method can include, after the retracting, locking the distal handle and the transfer stop such that the delivery catheter is slidably locked relative to the distal handle and the transfer stop. In some embodiments, the method can include releasing an elongate power lead connected to the blood pump from the proximal handle; and retracting the distal handle, the transfer stop, and the proximal handle proximally to remove the delivery sheath from the descending aorta. In some embodiments, the method can include electrically connecting an elongate power lead of the blood pump to an external control system through an electrical port of the proximal handle.

In another embodiment, a method for delivering a percutaneous blood pump to a target location in a blood vessel or body cavity is disclosed. The method can include: inserting a delivery sheath into the blood vessel; connecting a distal handle to a delivery sheath hub at a proximal end of the delivery sheath; advancing a transfer stop and a delivery catheter distally toward the distal handle to advance the blood pump to a distal portion of the delivery sheath within the blood vessel; and retracting the distal handle together with the transfer stop to cause the delivery sheath to retract proximally relative to the blood pump to deploy the blood pump at the target location in the blood vessel or body cavity.

In some embodiments, the method can include, before advancing the transfer stop and the delivery catheter distally, unlocking the distal handle such that the delivery catheter is slidable relative to the distal handle. In some embodiments, the method can include, before retracting the distal handle, unlocking the transfer stop such that the delivery catheter is slidable relative to the transfer stop. In some embodiments, advancing the transfer stop and the distal catheter comprises advancing the transfer stop distally until the transfer stop mates with the distal handle. In some embodiments, retracting the distal handle with the transfer stop comprises retracting the distal handle together with the transfer stop until the transfer stop mates with a proximal handle. In some embodiments, the method can include further retracting the distal handle, the transfer stop, and the proximal handle proximally to remove the delivery sheath from the body cavity or body lumen. In some embodiments, the method can include, before further retracting the distal handle, the transfer stop, and the proximal handle proximally, locking the distal handle and the transfer stop such that the delivery catheter is slidably locked relative to the distal handle and the transfer stop. In some embodiments, the percutaneous blood pump comprises an impeller disposed in a shroud and a plurality of struts extending from the shroud, wherein retracting the distal handle together with the transfer stop causes the delivery sheath to expose the blood pump in the blood vessel, the plurality of struts self-expanding upon the exposure.

In another embodiment, a method for delivering a percutaneous medical device to a target location in a body cavity or body lumen is disclosed. The method can include: inserting a delivery sheath into the body cavity or body lumen, a proximal end of the delivery sheath connectable to a distal handle; advancing a delivery catheter distally through the distal handle to position the medical device at a distal portion of the delivery sheath within the body cavity or body lumen; and retracting the distal handle proximally relative to the delivery catheter to retract the delivery sheath relative to the medical device to deploy the medical device at the target location.

In some embodiments, the method can include, after inserting the delivery sheath, connecting the distal handle to a delivery sheath hub at the proximal end of the delivery sheath. In some embodiments, the method can include, before advancing the delivery catheter distally, unlocking the distal handle such that the delivery catheter is slidable relative to the distal handle. In some embodiments, advancing the delivery catheter comprises advancing a transfer stop distally towards the distal handle with the transfer stop slidably locked relative to the delivery catheter. In some embodiments, the method can include, before retracting the distal handle, unlocking the transfer stop such that the delivery catheter is slidable relative to the transfer stop. In some embodiments, advancing the transfer stop comprises advancing the transfer stop distally until the transfer stop mates with the distal handle. In some embodiments, retracting the distal handle comprises retracting the distal handle together with the transfer stop until the transfer stop mates with a proximal handle. In some embodiments, the method can include further retracting the distal handle proximally to remove the delivery sheath from the body cavity or body lumen. In some embodiments, further retracting comprises retracting the distal handle, the transfer stop, and the proximal handle proximally. In some embodiments, the method can include, before further retracting the distal handle proximally, locking the distal handle and the transfer stop such that the delivery catheter is slidably locked relative to the distal handle and the transfer stop. In some embodiments, the method can include, before further retracting, releasing an elongate lead connected to the medical device from the proximal handle. In some embodiments, the method can include, before the further retracting, advancing a stiffening member to the medical device through the lumen of the lead. In some embodiments further retracting comprises anchoring the stiffening member while the distal handle is further retracted proximally. In some embodiments, inserting the delivery sheath comprises inserting the delivery sheath with an introducer dilator extending through the delivery sheath over a guidewire and into the body cavity or body lumen. In some embodiments, the medical device comprises a blood pump having an impeller disposed in a shroud and a plurality of struts extending from the shroud, wherein retracting the distal handle causes the delivery sheath to expose the blood pump in the body cavity or body lumen, the plurality of struts self-expanding upon the exposure. In some embodiments, the method can include electrically connecting an elongate power lead to a control system, the elongate power lead connected to the blood pump. In some embodiments, the method can include supplying power to the elongate power lead to impart rotation to the impeller to pump blood. In some embodiments, the method can include positioning the blood pump within a descending aorta such that an outlet of the pump is disposed at an elevation of the patient that is superior relative to an LI vertebral body. In some embodiments, the method can include positioning the blood pump within the descending aorta such that the outlet of the pump is disposed at an elevation of the descending aorta corresponding to an elevation between the LI vertebral body and a T10 vertebral body.

In another embodiment, a retrieval system for an intravascular blood pump is disclosed. The retrieval system can include: a retrieval dilator having a retrieval dilator hub and a clamping member at a proximal portion of the retrieval dilator, the retrieval dilator having a lumen sized and shaped to receive an elongate power lead of the intravascular blood pump therethrough, the clamping member having a clamped configuration in which the clamping member clamps against the elongate power lead and an unclamped configuration in which the elongate power lead is slidable relative to the clamping member; a retrieval sheath having a retrieval sheath hub at a proximal portion of the retrieval sheath, the retrieval sheath having a lumen sized and shaped to receive the retrieval dilator therethrough; and a retrieval handle having a lumen sized and shaped to receive the retrieval dilator therethrough such that the retrieval dilator extends through the retrieval handle and the retrieval sheath during a retrieval procedure, the retrieval handle having a distal connector configured to connect to the retrieval sheath hub and a proximal connector configured to connect to the retrieval dilator hub.

In some embodiments, the system can include a lead attachment device configured to attach to the elongate power lead during the retrieval procedure. In some embodiments, the lead attachment device comprises an elongate stiffening element and a locking element coupled to or formed with the elongate stiffening element, the elongate stiffening element and the locking element configured to be inserted into a lumen of the elongate power lead. In some embodiments, the system can include a guide rod, the lead attachment device connected to the guide rod. In some embodiments, the lead attachment device comprises a cuff sized to receive the elongate power lead therein. In some embodiments, the lead attachment device comprises one or a plurality of crimps configured to clamp over the cuff and the elongate power lead. In some embodiments, the system can include a support catheter slidable over the elongate power lead, the retrieval dilator slidable over the support catheter during the retrieval procedure.

In another embodiment, a retrieval system for a percutaneous blood pump is disclosed. The retrieval system can include a retrieval dilator having a clamping member at a proximal portion of the retrieval dilator, the retrieval dilator having a lumen sized and shaped to receive an elongate lead of the percutaneous blood pump therethrough, the clamping member having a clamped configuration in which the clamping member clamps against the elongate lead and an unclamped configuration in which the elongate lead is slidable relative to the clamping member; a retrieval sheath having a retrieval sheath hub at a proximal portion of the retrieval sheath, the retrieval sheath having a lumen sized and shaped to receive the retrieval dilator therethrough; and a retrieval handle having a lumen sized and shaped to receive the retrieval dilator therethrough.

In some embodiments, the retrieval dilator has a retrieval dilator hub, wherein the retrieval sheath has a retrieval sheath hub at a proximal portion of the retrieval sheath, the retrieval handle having a distal connector configured to connect to the retrieval sheath hub and a proximal connector configured to connect to the retrieval dilator hub. In some embodiments, the system can include a lead attachment device configured to attach to the elongate lead during a retrieval procedure. In some embodiments, the lead attachment device comprises an elongate stiffening element and a locking element coupled to or formed with the elongate stiffening element, the elongate stiffening element and the locking element configured to be inserted into a lumen of the elongate lead. In some embodiments, the system can include a guide rod, the lead attachment device connected to the guide rod. In some embodiments, the lead attachment device comprises a cuff sized to receive the elongate lead therein. In some embodiments, the lead attachment device comprises one or a plurality of crimps configured to clamp over the cuff and the elongate lead. In some embodiments, the system can include a support catheter slidable over the elongate lead, the retrieval dilator slidable over the support catheter during the retrieval procedure.

In another embodiment, a retrieval system for a percutaneous medical device is disclosed. The retrieval system can include a retrieval dilator having a clamping member at a proximal portion of the retrieval dilator, the retrieval dilator having a lumen sized and shaped to receive an elongate lead of the medical device therethrough, the clamping member having a clamped configuration in which the clamping member clamps against the elongate lead and an unclamped configuration in which the elongate lead is slidable relative to the clamping member; and a retrieval sheath having a lumen sized and shaped to receive the retrieval dilator therethrough.

In some embodiments, the system can include a retrieval handle having a lumen sized and shaped to receive the retrieval dilator therethrough. In some embodiments, the retrieval dilator has a retrieval dilator hub, wherein the retrieval sheath has a retrieval sheath hub at a proximal portion of the retrieval sheath, the retrieval handle having a distal connector configured to connect to the retrieval sheath hub and a proximal connector configured to connect to the retrieval dilator hub. In some embodiments, the system can include a lead attachment device configured to attach to the elongate lead during a retrieval procedure. In some embodiments, the lead attachment device comprises an elongate stiffening element and a locking element coupled to or formed with the elongate stiffening element, the elongate stiffening element and the locking element configured to be inserted into a lumen of the elongate lead. In some embodiments, the system can include a guide rod, the lead attachment device connected to the guide rod. In some embodiments, the lead attachment device comprises a cuff sized to receive the elongate lead therein. In some embodiments, the lead attachment device comprises one or a plurality of crimps configured to clamp over the cuff and the elongate lead. In some embodiments, the system can include a support catheter slidable over the elongate lead, the retrieval dilator slidable over the support catheter during the retrieval procedure.

In another embodiment, a method of retrieving an intravascular blood pump from a target location in a descending aorta is disclosed. The method can include connecting a retrieval handle with a retrieval sheath; inserting a retrieval dilator into the retrieval handle and through the retrieval sheath; advancing a stiffening member through a lumen of an elongate power lead of the intravascular blood pump; attaching a lead attachment device to the elongate power lead; anchoring the lead attachment device; advancing the retrieval dilator and the retrieval sheath over the elongate power lead to a location proximal the intravascular blood pump; clamping a clamping member of the retrieval dilator to the elongate power lead; disconnecting the retrieval dilator from the retrieval handle; advancing the retrieval sheath distally over the retrieval dilator; retracting the retrieval dilator to retract the intravascular blood pump into the retrieval sheath; and further retracting the retrieval dilator to retract the intravascular blood pump into the retrieval handle.

In some embodiments, attaching the lead attachment device to the elongate power lead comprises inserting a locking element into the elongate power lead to attach to the elongate power lead. In some embodiments, advancing the stiffening member comprises inserting an elongate stiffening element into the elongate power lead, the locking element coupled to or formed with the stiffening element. In some embodiments, anchoring the lead attachment device comprises anchoring the elongate stiffening element. In some embodiments, attaching the lead attachment device to the elongate power lead comprises inserting a cuff over the elongate power lead. In some embodiments, the method can include attaching the lead attachment device further comprises clamping a crimp on the cuff and the power lead. In some embodiments, the method can include inserting a guide rod into a support catheter, the cuff connected to the guide rod, wherein anchoring the lead attachment device comprises anchoring the guide rod. In some embodiments, the method can include advancing a support catheter over the power lead, wherein advancing the retrieval dilator and the retrieval sheath over the elongate power lead comprises advancing the retrieval dilator and the retrieval sheath over the support catheter. In some embodiments, the method can include disconnecting the retrieval handle from the retrieval sheath and delivering a second intravascular blood pump to the target location using the retrieval sheath. In some embodiments, the method can include, before attaching the lead attachment device to the elongate power lead, cutting a proximal portion of the elongate power lead to remove a connector from the elongate power lead.

In another embodiment, a method of retrieving a percutaneous blood pump from a target location in a patient is disclosed. The method can include: attaching a lead attachment device to an elongate lead of the blood pump; anchoring the lead attachment device; advancing a retrieval dilator and a retrieval sheath over the elongate lead; clamping a clamping member of the retrieval dilator to the elongate lead; retracting the retrieval dilator to retract the blood pump into the retrieval sheath; and further retracting the retrieval dilator to retract the blood pump into a retrieval handle coupled with the retrieval sheath.

In some embodiments, the method can include inserting the retrieval dilator into the retrieval handle and through the retrieval sheath; and connecting the retrieval dilator to the retrieval handle. In some embodiments, the method can include, before the retracting, disconnecting the retrieval dilator from the retrieval handle. In some embodiments, attaching the lead attachment device to the elongate lead comprises inserting a locking element into the elongate lead to attach to the elongate lead. In some embodiments, advancing the stiffening member comprises inserting an elongate stiffening element into the elongate lead, the locking element coupled to or formed with the stiffening element. In some embodiments, anchoring the lead attachment device comprises anchoring the elongate stiffening element. In some embodiments, attaching the lead attachment device to the elongate lead comprises inserting a cuff over the elongate lead. In some embodiments, attaching the lead attachment device further comprises clamping a crimp on the cuff and the lead. In some embodiments, the method can include inserting a guide rod into a support catheter, the cuff connected to the guide rod, wherein anchoring the lead attachment device comprises anchoring the guide rod. In some embodiments, the method can include advancing a support catheter over the lead, wherein advancing the retrieval dilator and the retrieval sheath over the lead comprises advancing the retrieval dilator and the retrieval sheath over the support catheter. In some embodiments, the method can include disconnecting the retrieval handle from the retrieval sheath and delivering a second percutaneous blood pump to the target location using the retrieval sheath. In some embodiments, the method can include, before attaching the lead attachment device to the elongate lead, cutting a proximal portion of the elongate lead to remove a connector from the elongate lead.

In another embodiment, a method of retrieving a percutaneous medical device from a target location in a patient is disclosed. The method can include: advancing a retrieval dilator and a retrieval sheath over an elongate lead of the medical device; clamping a clamping member of the retrieval dilator to the elongate lead; and retracting the retrieval dilator to retract the medical device into the retrieval sheath.

In some embodiments, the method can include attaching a lead attachment device to an elongate lead of the medical device and anchoring the lead attachment device. In some embodiments, the method can include inserting the retrieval dilator into a retrieval handle and through the retrieval sheath; and connecting the retrieval dilator to the retrieval handle. In some embodiments, the method can include further retracting the retrieval dilator to retract the medical device into the retrieval handle. In some embodiments, the method can include, before the retracting, disconnecting the retrieval dilator from the retrieval handle. In some embodiments, attaching the lead attachment device to the elongate lead comprises inserting a locking element into the elongate lead to attach to the elongate lead. In some embodiments, advancing the stiffening member comprises inserting an elongate stiffening element into the elongate lead, the locking element coupled to or formed with the stiffening element. In some embodiments, anchoring the lead attachment device comprises anchoring the elongate stiffening element. In some embodiments, attaching the lead attachment device to the elongate lead comprises inserting a cuff over the elongate lead. In some embodiments, attaching the lead attachment device further comprises clamping a crimp on the cuff and the lead. In some embodiments, the method can include inserting a guide rod into a support catheter, the cuff connected to the guide rod, wherein anchoring the lead attachment device comprises anchoring the guide rod. In some embodiments, the method can include advancing a support catheter over the lead, wherein advancing the retrieval dilator and the retrieval sheath over the lead comprises advancing the retrieval dilator and the retrieval sheath over the support catheter. In some embodiments, the method can include disconnecting the retrieval handle from the retrieval sheath and delivering a second medical device to the target location using the retrieval sheath. In some embodiments, the method can include, before attaching the lead attachment device to the elongate lead, cutting a proximal portion of the elongate lead to remove a connector from the elongate lead.

In another embodiment, a blood flow assist system is disclosed. The blood flow assist system can include an impeller disposed in a pump housing of a pump; and a support structure comprising a plurality of struts coupled with the pump housing, each strut having a contact element at a distal portion thereof, the contact element configured to at least intermittently contact a blood vessel wall to maintain spacing of the pump housing from a blood vessel wall in which the pump housing is disposed, the contact element comprising a contact surface having a contoured profile to facilitate contact with the blood vessel wall.

In some embodiments, the contoured profile comprises an opening in the contact surface. In some embodiments, the contoured profile comprises a plurality of fingers spaced apart by the opening. In some embodiments, the contoured profile comprises a projection extending outwardly from the contact surface.

In another embodiment, a blood flow assist system can include: an impeller disposed in a pump housing of a pump; and a support structure comprising a plurality of struts coupled with the pump housing, each strut having a contact element at a distal portion thereof and an elongate member extending between the contact element and the pump housing along an axis of the strut, the elongate member having a cross-section taken perpendicular to the axis of the strut that varies rotationally along a length of the strut.

In some embodiments, the elongate member is twisted about the axis of the strut.

In another embodiment, a blood flow assist system can include an impeller disposed in a pump housing of a pump, the pump comprising a longitudinal axis; and a support structure comprising a plurality of struts coupled with the pump housing, each strut comprising a plurality of segments integrally formed and connected with one another, the plurality of segments comprising a first segment extending distally and radially outwardly relative to the longitudinal axis and a second segment extending proximally and radially outwardly from a distal end of the first segment, the second segment configured to at least intermittently engage a blood vessel wall.

In some embodiments, the first and second segments meet at a joint, wherein, in a collapsed configuration, the joint of each strut is disposed radially inward within a sheath.

In another embodiment, a blood flow assist system can include: an impeller disposed in a pump housing of a pump, the pump comprising a longitudinal axis; and a support structure comprising a plurality of struts coupled with the pump housing, each strut having a contact element at a distal end thereof, the contact element configured to at least intermittently contact a wall of a blood vessel, the support structure having an expanded diameter in an expanded configuration, the expanded diameter less than a diameter of the blood vessel.

In some embodiments, each strut extends radially and distally outward from the pump housing.

In another embodiment, a blood flow assist system can include an impeller disposed in a pump housing of a pump, the pump comprising a longitudinal axis; and a support structure comprising a strut coupled with the pump housing, the strut having a contact element at a distal end thereof, the contact element configured to at least intermittently contact a wall of a blood vessel, the strut being at least partially revolved about the longitudinal axis.

In some embodiments, the strut is disposed about the longitudinal axis in a helical profile. In some embodiments, the strut comprises a coiled spring.

In another embodiment, a blood flow assist system can include an impeller disposed in a pump housing of a pump, the pump comprising a longitudinal axis; a support structure comprising a first plurality of struts coupled with and extending proximally from the pump housing and a second plurality of struts coupled with and extending distally from the pump housing, each strut of the first and second pluralities of struts having a contact element at a distal end thereof, the contact element configured to at least intermittently contact a wall of a blood vessel; and a retrieval feature coupled with the support structure to facilitate collapse of at least the first plurality of struts.

In some embodiments, the retrieval feature comprises a slip ring disposed about the first plurality of struts and a snare feature coupled to or formed with the slip ring.

In another embodiment, a blood flow assist system can include an impeller disposed in a pump housing of a pump; and a support structure comprising a plurality of struts coupled with the pump housing, each strut having a contact element at a distal end thereof, the contact element configured to at least intermittently contact a wall of a blood vessel, the plurality of struts comprising a first strut and a second strut extending distally from the pump housing, the support structure further comprising a brace distal the pump housing, the brace extending between and mechanically connected to the first strut and the second strut.

In some embodiments, the brace comprises a first distally-extending segment extending from the first strut and a second distally-extending segment extending from the second strut, the first and second distally-extending segments joined at a connection location. In some embodiments, the brace comprises an arch brace having a curved profile extending between the first and second struts.

In another embodiment, a blood flow assist system can include: an impeller assembly comprising a rotor assembly and an impeller coupled with the rotor assembly, the rotor assembly comprising a concave bearing surface; and a drive unit proximal the impeller assembly, the drive unit comprising a drive magnet and a drive bearing between the drive magnet and the impeller assembly, the drive bearing comprising a convex bearing surface shaped to fit within the concave bearing surface, the convex bearing surface comprising a plurality of distally-projecting segments extending from a base of the drive bearing, the plurality of distally-projecting segments spaced apart circumferentially to define at least one channel between adjacent segments, the drive bearing comprising a curved or ramped surface angled distally from the base and defining a portion of the at least one channel.

In some embodiments, the distally-extending projections extend distal the curved or ramped surface. In some embodiments, a distal end of the drive unit is disposed distal of a proximal end of the rotor assembly. In some embodiments, the rotor assembly comprises an impeller shaft and a rotor magnet coupled to the impeller shaft, the impeller disposed on the impeller shaft. In some embodiments, the impeller assembly comprises a second impeller disposed on the impeller shaft spaced apart proximally from the impeller along the impeller shaft. In some embodiments, the system can include a flange extending non-parallel from a proximal end portion of the impeller shaft, the second impeller comprising a plurality of vanes disposed on a generally proximally-facing surface of the flange. In some embodiments, the blood flow assist system comprises a percutaneous pump configured for percutaneous insertion to a treatment location within a body of a patient. In some embodiments, the system can include a motor mechanically coupled with the drive magnet and a power wire connected to the motor, the power wire extending proximally from the motor.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1A is a schematic perspective, partially-exploded view of a blood flow assist system, according to various embodiments.

FIG. 1B is a schematic perspective view of a pump at a distal portion of the blood flow assist system of FIG. 1A.

FIG. 1C is a schematic perspective, partially-exploded view of the pump of FIG. 1B.

FIG. 1D is a schematic side sectional view of a motor housing according to various embodiments.

FIG. 1E is a schematic perspective view of a motor and a motor mount support.

FIG. 1F is a schematic perspective view of a distal end of a power lead having lumens shaped to received conductors that are configured to supply power to the motor.

FIG. 1G is a schematic perspective view of a proximal end portion of the power lead.

FIG. 1H is a schematic side view of the pump disposed in a collapsed configuration in a delivery sheath.

FIG. 1I is a schematic perspective view of a retrieval feature used to remove the pump, according to some embodiments.

FIG. 1J is a cross-sectional view of an alternative embodiment in which a drive shaft is coupled to a motor configured to be disposed outside the patient when the pump is in use.

FIG. 1K is a schematic side view of a connector at a proximal portion of a power lead according to various embodiments.

FIG. 1L is a schematic perspective view of a plurality of elongate conductors extending through the power lead.

FIG. 1M is a schematic side view of a proximal end portion of the power lead of FIG. 1K.

FIG. 1N is a schematic side sectional view of the proximal end portion shown in FIG. 1M.

FIG. 1O is a schematic perspective view of at least a portion of a percutaneous medical system, according to various embodiments.

FIG. 2A illustrates an introducer set, according to various embodiments.

FIG. 2B illustrates a proximal portion of a delivery sheath of the introducer set of FIG. 2A.

FIG. 2C is an image showing a guidewire after insertion into a descending aorta of a patient.

FIG. 2D is an image showing the delivery sheath and a dilator inserted into the descending aorta.

FIG. 2E is an image showing the delivery sheath after removal of the dilator and guidewire.

FIG. 2F illustrates a delivery system for a percutaneous medical device, e.g., an intravascular blood pump, in an unlocked configuration, according to various embodiments.

FIG. 2G shows a distal handle of the delivery system in a locked configuration.

FIG. 2H shows the delivery system of FIG. 2F in a locked configuration.

FIG. 2I illustrates a proximal end portion of the delivery sheath.

FIG. 2J illustrates the distal handle of FIGS. 2F and 2G and the proximal end portion of the delivery sheath prior to connection.

FIG. 2K illustrates the distal handle of FIGS. 2F and 2G connected to the proximal end portion of the delivery sheath.

FIG. 2L illustrates the distal handle of FIGS. 2F and 2G in the locked configuration.

FIG. 2M illustrates the distal handle of FIGS. 2F and 2G being moved to the unlocked configuration.

FIG. 2N illustrates a transfer stop of the delivery system of FIG. 2F before being advanced distally towards the distal handle.

FIG. 2O illustrates the transfer stop after being advanced distally to mate with the distal handle.

FIG. 2P is an image showing the position of a blood pump within the delivery sheath after performing the step shown in FIG. 2O.

FIG. 2Q shows the transfer stop being moved to an unlocked configuration.

FIG. 2R shows the distal handle and transfer stop being moved to mate with a proximal handle of the delivery system.

FIG. 2S is an image showing the blood pump deployed in the descending aorta after the delivery sheath is retracted proximally as illustrated in FIG. 2R.

FIG. 2T is an image showing the blood pump deployed in the descending aorta after the distal handle and transfer stop are retracted to the position shown in FIG. 2R to retract the delivery sheath.

FIG. 2U illustrates the insertion of a guidewire through the delivery system to the pump.

FIG. 2V illustrates unlocking of a power lead connected to the pump with the proximal handle.

FIG. 2W illustrates releasing the power lead with a proximal release knob.

FIG. 2X illustrates a technique for removing the delivery system from the body while leaving the pump in place.

FIG. 3A is a schematic side exploded view of the delivery system of FIG. 2F.

FIG. 3B is a schematic side sectional view of the distal handle.

FIG. 3C is a schematic side view of a distal portion of the delivery catheter.

FIG. 3D is a schematic side sectional view of the distal portion of the delivery catheter of FIG. 3C, taken along section 3D-3D.

FIG. 3E is a schematic side view of a portion of the delivery catheter.

FIG. 3F is a schematic perspective exploded view of the proximal handle.

FIG. 3G is a schematic side sectional view of the proximal handle of FIG. 3F.

FIG. 3H is an enlarged schematic side sectional view of the proximal handle of FIG. 3G.

FIG. 3I is a schematic side view of a locking pin assembly, according to various embodiments.

FIG. 3J is a top view of a plunger of the proximal handle, according to various embodiments.

FIG. 3K is a side view of the plunger of FIG. 3J.

FIGS. 3L and 3M are perspective views of a lead release actuator.

FIG. 3N is a schematic perspective exploded view of the distal handle.

FIG. 3O is a schematic perspective exploded view of the transfer stop.

FIG. 3P illustrates an example of the proximal handle that includes one or more first vent holes and one or more second vent holes.

FIG. 3Q illustrates the proximal handle of FIG. 3P after the lead has been released from the proximal handle.

FIG. 4A illustrates a retrieval system, according to various embodiments.

FIG. 4B illustrates a lead attachment device, according to one embodiment.

FIG. 4C illustrates an arrangement of components of the retrieval system of FIG. 4A prior to retrieval.

FIG. 4D illustrates the connection of a retrieval handle to a retrieval sheath hub.

FIG. 4E illustrates the insertion of a retrieval dilator into the retrieval handle.

FIG. 4F illustrates the connection of the retrieval handle to a retrieval dilator hub of the retrieval dilator.

FIG. 4G illustrates the insertion of a guide rod into a support catheter, with the lead attachment device extending from the support catheter.

FIG. 4H illustrates the insertion of a guidewire into a power lead that is connected to an intravascular pump.

FIG. 4I is an image showing the positioning of the guidewire within the power lead and advanced to the pump.

FIG. 4J illustrates a connection between the lead attachment device and the power lead.

FIG. 4K illustrates the connection between the lead attachment device and the power lead after crimping the lead with a plurality of crimps.

FIG. 4L is an image showing the advancement of the support catheter over the guidewire to a location proximal the pump.

FIG. 4M is an image showing the retrieval dilator and retrieval sheath advanced over the support catheter.

FIG. 4N is an image showing the retrieval sheath advanced to a location proximal the pump.

FIG. 4O illustrates a retrieval system, according to another embodiment.

FIG. 4P illustrates an arrangement of components of the retrieval system of FIG. 4O prior to retrieval.

FIG. 4Q is a schematic illustration of the lead attachment device shown in the embodiment of FIG. 4B.

FIG. 4R is a schematic illustration of the lead attachment device shown in the embodiment of FIG. 4O.

FIG. 4S is a schematic perspective exploded view of the retrieval handle, according to various embodiments.

FIG. 4T is a schematic side sectional view of the retrieval handle of FIG. 4S.

FIG. 5A is an image showing a front perspective view of a localization system, according to one embodiment.

FIG. 5B is a schematic side view of the localization system of FIG. 5A.

FIG. 5C is a schematic plan view of a laser cut pattern for the localization system of FIG. 5B.

FIG. 5D is a schematic side plan view of a strut having a dome- or spherical-shaped contact pad.

FIG. 5E is a schematic perspective view of a contact pad that pillows into a blood vessel wall, according to some embodiments.

FIG. 5F is a schematic front sectional view of the contact pad shown in FIG. 5E.

FIG. 5G is a schematic side sectional view of the contact pad shown in FIG. 5E.

FIG. 5H is a schematic plan view of a contact element of a strut that can provide for improved pillowing into a blood vessel wall, according to various embodiments.

FIG. 5I is a schematic plan view of a contact element of a strut that can provide for improved pillowing into a blood vessel wall, according to various embodiments.

FIG. 5J is a schematic plan view of a contact element of a strut that can provide for improved pillowing into a blood vessel wall, according to various embodiments.

FIG. 5K is a schematic perspective view of a shroud having a plurality of twisted struts extending therefrom.

FIG. 5L is an axial end view of the struts of FIG. 5K.

FIG. 6A is an image of a front perspective of a localization system according to another embodiment.

FIG. 6B is an image of a side view of the localization system of FIG. 6A.

FIG. 6C is a schematic side view of the localization system of FIGS. 6A-6B.

FIG. 6D is a schematic enlarged view of the second end of the strut of FIGS. 6A-6C.

FIGS. 6E and 6F are schematic plan views of the localization system in a laser cut pattern prior to assembly.

FIG. 6G illustrates a plan view of a distal end of the strut of FIG. 6D.

FIG. 6H is a schematic side view of a plurality of struts in expanded and collapsed configurations relative to a sheath.

FIG. 6I is a schematic side view of an example strut engaging a vessel wall.

FIG. 6J is a front view of the struts disposed within the sheath.

FIG. 6K illustrates a strut having a curved expanded profile.

FIGS. 7A-7E show a method of delivering and deploying a localization and positioning system that incorporates struts with contact elements, a tether, and propulsive force.

FIG. 8A is a schematic perspective view of a localization system in a collapsed configuration, according to another embodiment.

FIG. 8B is a schematic plan view of a laser cut design for the system of FIG. 8A.

FIG. 9 is a schematic side view of a plurality of struts according to various embodiments.

FIGS. 10A-10D illustrate an example of a first plurality of struts extending from the shroud and a second plurality of struts extending from the shroud, according to various embodiments.

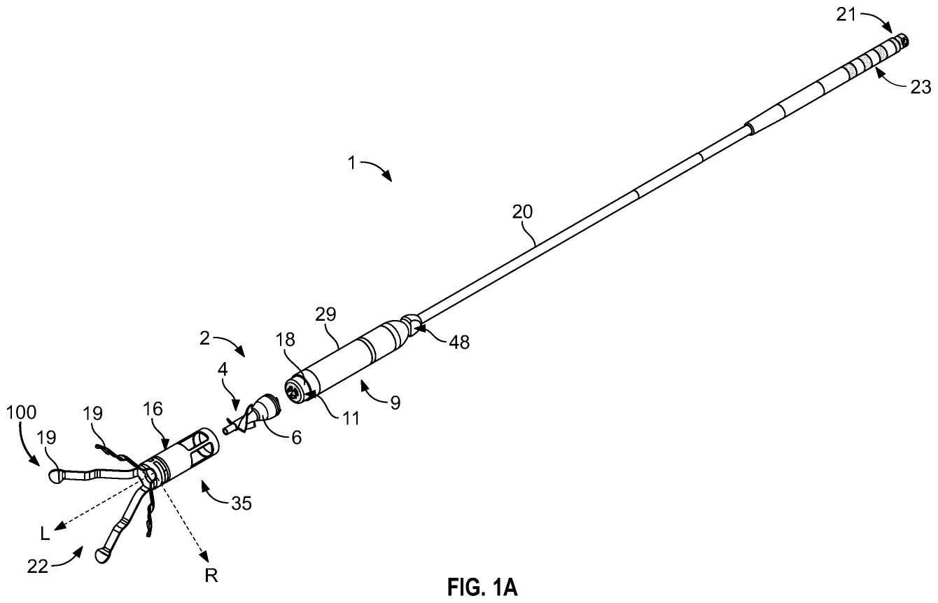

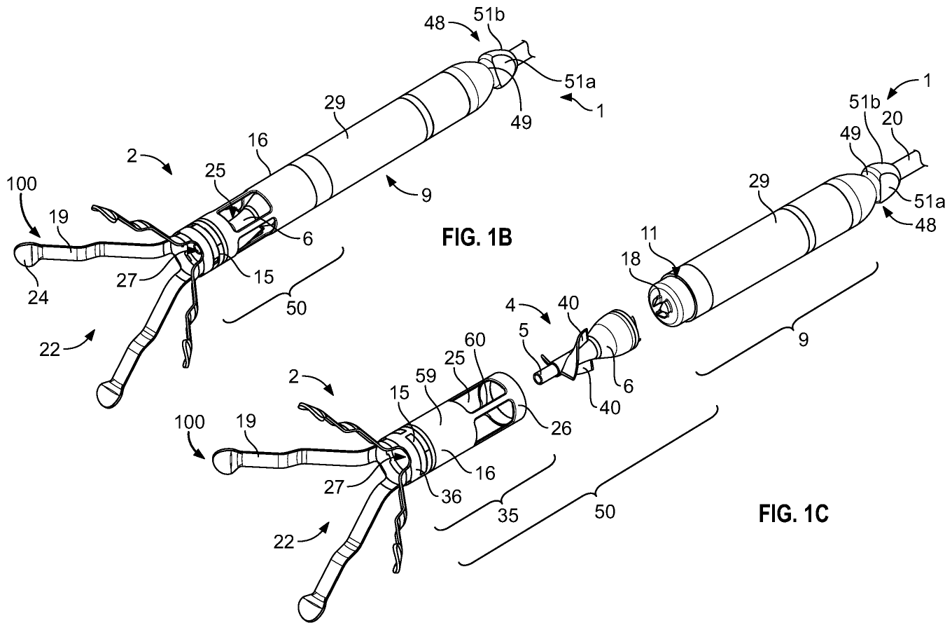

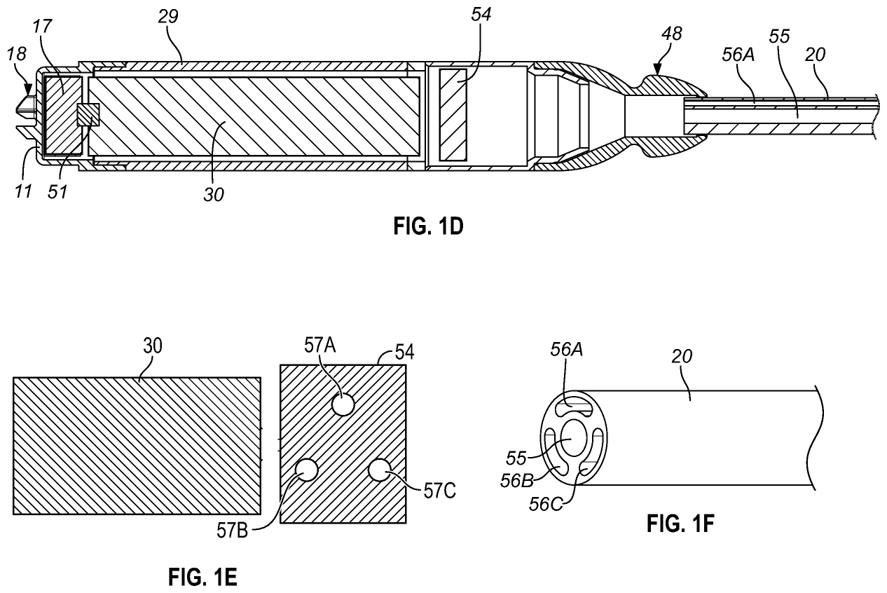

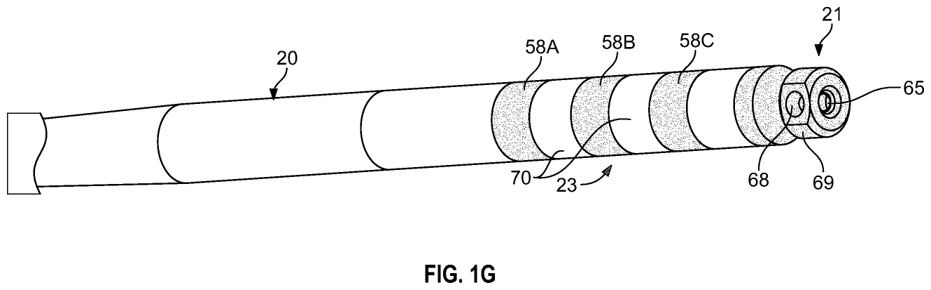

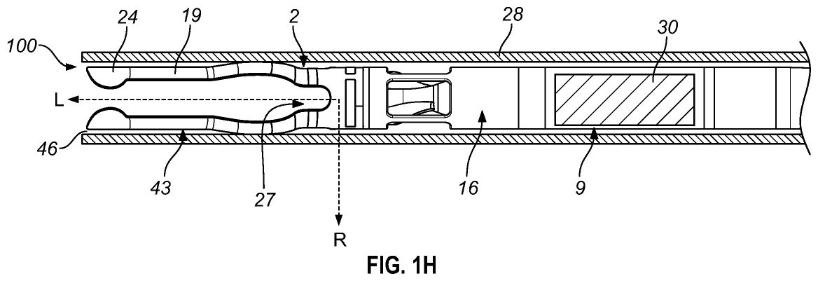

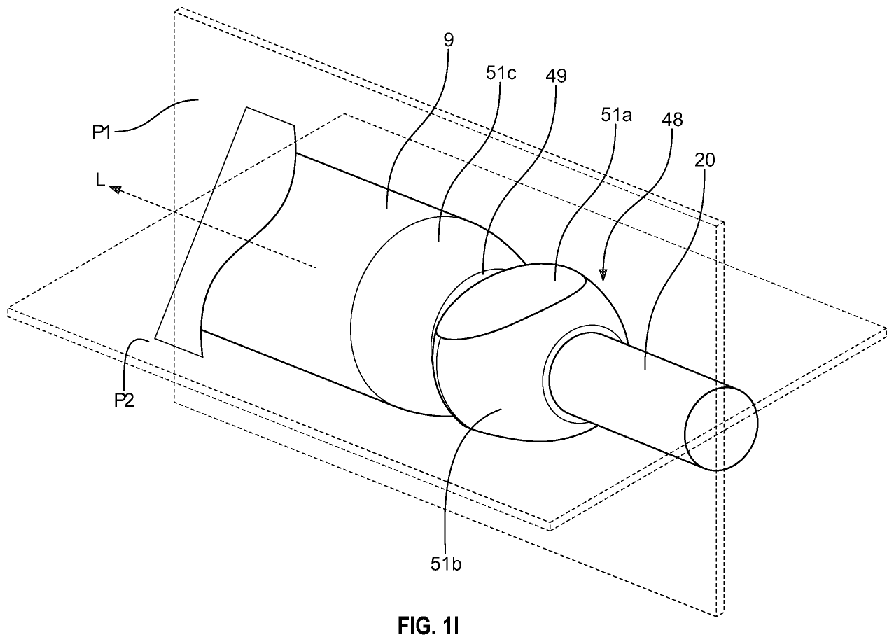

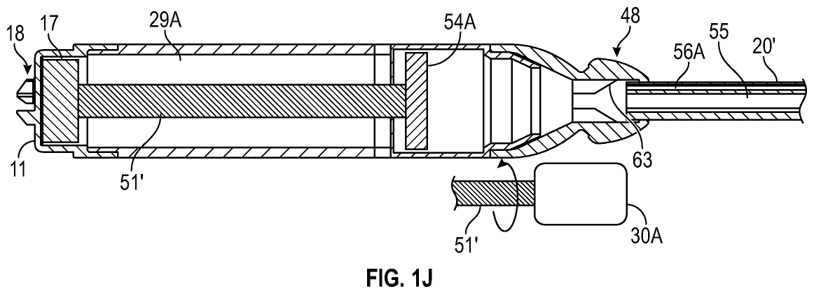

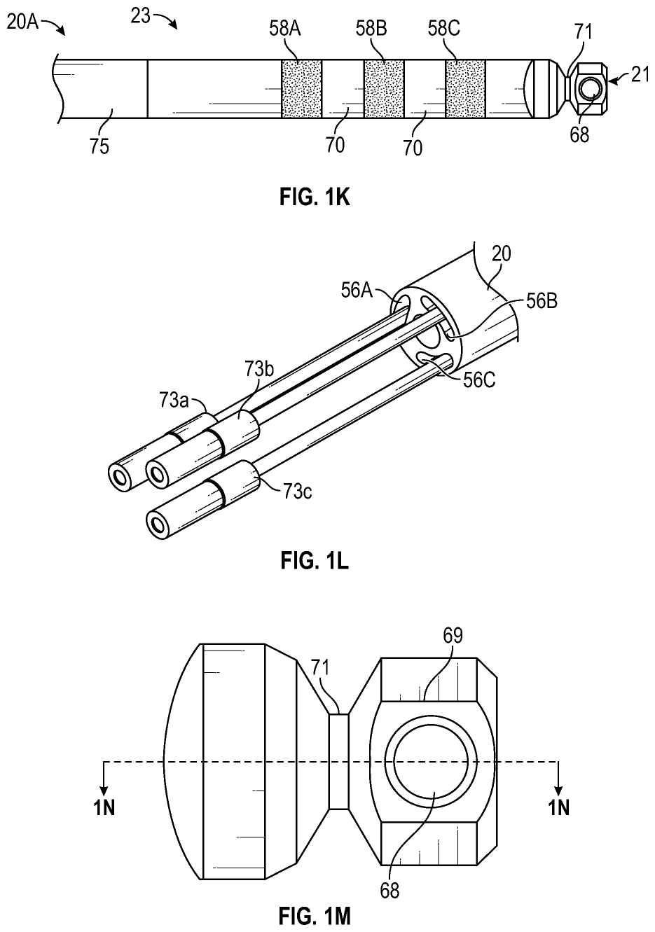

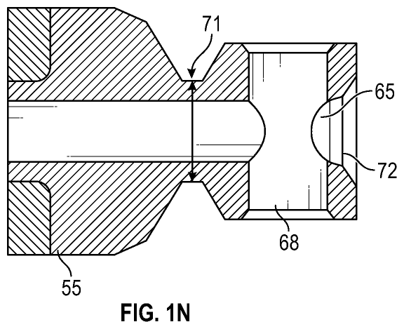

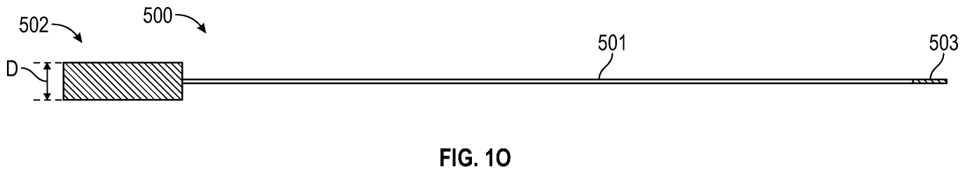

FIG. 1OE is a two-dimensional unwrapped view of the struts 10A-10D.

FIGS. 11A-11B illustrate a plurality of struts extending from the shroud, according to another embodiment.

FIGS. 12A-12B illustrates a support structure comprising a strut shaped in a curved or coiled shape.

FIGS. 13A-13C illustrate a pump having a shroud with a first plurality of struts extending proximally from the shroud and a second plurality of struts extending distally from the shroud.

FIGS. 14A-14B illustrate a pump having a shroud with a plurality of struts connected by brace members, according to various embodiments.

FIG. 15A is a schematic perspective view of a drive bearing according to various embodiments.

FIG. 15B is a front end view of the drive bearing of FIG. 15A.

FIG. 15C is a side view of the drive bearing of FIG. 15A.

FIG. 15D is a schematic front end view of a drive bearing according to another embodiment.

FIG. 15E is a schematic front end view of a drive bearing according to another embodiment.

FIGS. 15F and 15G illustrate additional examples of drive units according to various embodiments.

FIG. 16A is a schematic perspective view of an integrated rotor core comprising an impeller shaft with flow tube and a secondary impeller.

FIG. 16B is a schematic perspective view of a proximal portion of the integrated rotor core of FIG. 16A.

FIG. 16C is a sectional view taken along the longitudinal axis of the rotor core of FIG. 16B.

FIG. 16D is a schematic proximal end view of the integrated rotor core of FIG. 16C.

FIG. 17A is a schematic perspective, exploded view of a segmented cone bearing comprising a proximal portion of the integrated rotor core and the drive bearing.

FIG. 17B is a distal end sectional view of the secondary impeller and drive bearing.

### DETAILED DESCRIPTION

Refer now to the drawings wherein depicted elements are not necessarily shown to scale and wherein like or similar elements are designated by the same reference numeral through the several views.

Referring to the drawings in general, it will be understood that the illustrations are for the purpose of describing particular implementations of the disclosure and are not intended to be limiting thereto. While most of the terms used herein will be recognizable to those of ordinary skill in the art, it should be understood that when not explicitly defined, terms should be interpreted as adopting a meaning presently accepted by those of ordinary skill in the art.

Intravascular medical procedures allow numerous possibilities for therapy with many benefits and advantages over open procedures. Devices and methods used to access the vasculature and close the access point after therapy are known in the art in connection with the Seldinger technique. Intravascular procedures can be divided into those that take place in the venous system and those that take place in the arterial system. Procedures on the arterial side are made more challenging by the higher blood pressure and smaller vessel size (than corresponding veins). Intravascular procedures can also be divided into those that leave a device behind and those that do not. Stents are typical devices that are left behind. Devices that are placed in the vasculature in intravascular procedures may be passive (like stents) or active (powered devices like blood pumps). Active devices may have batteries or power leads that pass through the wall of the blood vessel. Once outside the blood vessel, such power leads may be connected to components implanted in the body or may pass through the skin to the outside of the body.

Intravascular procedures that place an active device in the arterial system and have a power lead passing through the wall of the artery are atypical medical devices. The challenges of small vessel size and high blood pressure should be accounted for in such procedures. One such device is a catheter-deployed blood pump, also known as a percutaneous mechanical circulatory support (pMCS) device. Intravascular pump systems disclosed herein can be used for temporary or long-term cardiac or renal support in patients indicated for heart failure, post-myocardial infarction, and other heart-related illnesses.

### I. Overview of Blood Flow Assist Systems

Various embodiments disclosed herein include systems and methods for placing, adjusting, and removing arterial devices (e.g., blood flow assist systems 1) with indwelling elongate leads (e.g., power leads 20 that pass through the artery wall). The system can further comprise a plurality of components: an introducer set (IS), a deployment or delivery system (DS), and a retrieval system (RS) for introducing a delivery sheath to the vasculature, deploying the arterial device, and retrieving the arterial device, respectively. The embodiments illustrated and described herein relate to a blood flow assist system, such as an intravascular blood pump, but it should be appreciated that various components may be used with other types of medical devices. For example, other types of medical devices may utilize any combination of the elongate lead(s) 20, the introducer set (IS), the deployment or delivery system (DS), and/or the retrieval system (RS). For example, other types of percutaneous medical devices may utilize the components and methods described herein.

Various embodiments disclosed herein relate to a blood flow assist system 1 configured to provide circulatory support to a patient, as illustrated in FIGS. 1A-1J. The system 1 can be sized for intravascular delivery to a treatment location within the circulatory system of the patient, e.g., to a location within the descending aorta of the patient. As shown in FIG. 1A, the system 1 can have a proximal end 21 with a connector 23 configured to connect to an external control system, e.g., a console (not shown). The connector 23 can provide electrical communication between the control system and an elongate lead (e.g., an elongate power lead 20) extending distally along a longitudinal axis L from the connector 23 and the proximal end 21. The connector 23 can be disposed at a proximal portion of the lead 20. The power lead 20 can comprise an elongate body that electrically and mechanically connects to a pump 2 at or near a distal end 22 of the blood flow assist system 1, with the distal end 22 spaced apart from the proximal end 21 (which can also serve as a proximal end of the lead 20) along the longitudinal axis L. As explained herein, the power lead 20 can also serve as a flexible tether configured to oppose loads applied in opposite directions at opposite ends of the power lead 20.

The pump 2 can comprise a pump head 50 including a pump housing 35 connected to a drive unit 9 that includes a motor housing 29. A retrieval feature 48 can be provided at a proximal end portion of the pump 2. In some embodiments, the retrieval feature can be coupled with the distal end of the power lead 20 between the power lead 20 and the motor housing 29. After a procedure, the clinician can remove the pump 2 from the patient by engaging a tool (e.g., a snare, a clamp, hook, etc.) with the retrieval feature 48 to pull the pump 2 from the patient. For example, the retrieval feature 48 can comprise a neck 49 (e.g., a reduced diameter section) at a proximal curved portion 51c of the motor housing 29 and an enlarged diameter section disposed proximal the neck 49. The enlarged diameter section can comprise a first curved portion 51a and a second curved portion 51b, as shown in FIGS. 1B, 1C, and 1I. The first and second curved portions 51a, 51b can comprise convex surfaces, e.g., convex ball portions. The first and second curved portions 51a, 51b can have different radii of curvature. For example, as shown in FIG. 1I, the first curved portion 51a can have a larger radius of curvature than the second curved portion 51b. The first curved portion 51a can be disposed on opposing sides of the retrieval feature 48 in some embodiments. The second curved portion 51b can be disposed around the first curved portion 51a and can have a radially-outward facing surface and a proximally-facing convex surface coupled to the distal end of the power lead 20. The neck 49 can have a first depth at a first circumferential position of the retrieval feature 48 and a second depth less than the first depth at a second circumferential position of the retrieval feature 48 spaced apart from the first circumferential position.

Beneficially, as shown in FIG. 1I, one or more first planes P1 extending parallel to the longitudinal axis L and intersecting the first curved portion 51a can have a first angle or taper between the proximal curved portion 51c of the motor housing 29 and the first curved portion 51a. One or more second planes P2 extending parallel to the longitudinal axis L and intersecting the second curved portion 51b can have a second angle or taper (which is different from the first angle or taper) between the proximal curved portion 51c of the motor housing 29 and the second curved portion 51b. The first angle or taper can provide a gradual, continuous (generally monotonically decreasing) geometric transition between the proximal curved portion 51c of the motor housing 29 and the power lead 20, which can provide for smooth blood flow and reduce the risk of thrombosis. The second curved portion 51b can serve as a lobe that extends radially outward, e.g., radially farther out than the first curved portion 51a. The second curved portion 51b can be used to engage with a retrieval device or snare to remove the pump 2 from the anatomy. Some cross sections through the longitudinal axis of the retrieval feature 48 can contain a substantial neck (e.g., a local minimum in the radius of curvature measured along its central axis) while other cross sections through the longitudinal axis of the retrieval feature 48 can contain an insubstantial local minimum or no local minimum. In the illustrated embodiment, there are two first curved portions 51a that can serve as a dual lobe retrieval feature. In other embodiments, more or fewer lobes can be provided to enable pump retrieval while ensuring smooth flow transitions between the motor housing 29 and power lead 20.

As shown in FIGS. 1B-1C, IE, and 1I, the neck 49 can be disposed between the curved portions 51a, 51b and a proximally-facing convex surface 51c of the motor housing 29. In the illustrated embodiment, the retrieval feature 48 can be coupled to or integrally formed with the motor housing 29. In other arrangements, the retrieval feature 48 can be disposed at other locations of the pump 2. As shown, the retrieval feature 48 can be symmetrical and continuously disposed about the longitudinal axis L. In other arrangements, the retrieval feature 48 can comprise a plurality of discrete surfaces spaced apart circumferentially and/or longitudinally. In the illustrated embodiments, the motor housing 29 (and motor) can be part of the pump 2 and disposed inside the vasculature of the patient in use. In other embodiments, however, the motor housing 29 (and motor) can be disposed outside the patient and a drive cable can connect to the impeller 6.

As shown in FIGS. 1A-1C, the drive unit 9 can be configured to impart rotation to an impeller assembly 4 disposed in the pump housing 35 of the pump head 50. As explained herein, the drive unit 9 can include a drive magnet 17 (see FIG. 1D) and a motor 30 (sec FIGS. 1D-1E) disposed in the motor housing 29 capped by a distal drive unit cover 11. The motor 30 is shown schematically in FIG. 1D. The drive unit cover 11 can be formed with or coupled to a drive bearing 18. The drive magnet 17 can magnetically couple with a corresponding driven or rotor magnet (not shown) of the impeller assembly 4 that is disposed proximal the impeller 6 within the shroud 16. The power lead 20 can extend from the treatment location to outside the body of the patient, and can provide electrical power (e.g., electrical current) and/or control to the motor 30. Accordingly, no spinning drive shaft extends outside the body of the patient in some embodiments. As explained herein, the power lead 20 can energize the motor 30, which can cause the drive magnet 17 to rotate about the longitudinal axis L, which can serve as or be aligned with or correspond to an axis of rotation. Rotation of the drive magnet 17 can impart rotation of the rotor magnet and a primary or first impeller 6 of the impeller assembly 4 about the longitudinal axis L. For example, as explained herein, the rotor magnet (which can be mechanically secure to an impeller shaft 5) can cause the impeller shaft 5 (which can serve as a flow tube) and the first impeller 6 to rotate to pump blood. In other embodiments, the drive unit 9 can comprise a stator or other stationary magnetic device. The stator or other magnetic device can be energized, e.g., with alternating current, to impart rotation to the rotor magnet. In the illustrated embodiments, the impeller 6 can have one or a plurality of blades 40 extending radially outward along a radial axis R that is radially transverse to the longitudinal axis L. For example, the first impeller 6 can have a plurality of (e.g., two) longitudinally-aligned blades 40 that extend radially outwardly from a common hub and that have a common length along the longitudinal axis L. The curvature and/or overall profile can be selected so as to improve flow rate and reduce shear stresses. Skilled artisans would appreciate that other designs for the first impeller 5 may be suitable.

As shown in FIGS. 1A-1C, the impeller assembly 4 can be disposed in a shroud 16. The impeller shaft 5 can be supported at a distal end by a sleeve bearing 15 connected to a distal portion of the shroud 16. A support structure such as a localization system 100 (discussed further below) can comprise a base portion 36 coupled with the sleeve bearing 15 and/or the shroud 16. In some embodiments, the base portion 36, the sleeve bearing 15, and/or the shroud 16 can be welded together. In other embodiments, the sleeve bearing 15 and/or the shroud 16 can be formed as one part. The base portion 36 of the support structure or localization system 100 (which can be part of or serve as a support structure), the sleeve bearing 15, and the shroud 16 can cooperate to at least partially define the pump housing 35, as shown in FIGS. 1A and 1C. The localization system 100 can comprise a plurality of self-expanding struts 19 having convex contact pads 24 configured to contact a blood vessel wall to maintain spacing of the pump housing 35 from the wall of the blood vessel in which the pump housing 35 is disposed. In FIGS. 1A-1C, the struts 19 of the localization system 100 are illustrated in an expanded, deployed configuration, in which the contact pads 24 extend radially outward to a position in which the contact pads 24 would contact a wall of a blood vessel within which the pump 2 is disposed to at least partially control position and/or orientation of the pump head 50 relative to the blood vessel wall, e.g., to anchor, the pump 2 during operation of the system 1.

A first fluid port 27 can be provided distal the impeller assembly 4 at a distal end of the pump housing 35. The shroud 16 can comprise a proximal ring 26 coupled with the motor housing 29 and a plurality of second fluid ports 25 formed in a proximal portion of the shroud 16 adjacent (e.g., immediately distal) the proximal ring 26. As shown in FIG. 1C, the second fluid ports 25 can comprise openings formed between axially-extending members 60 (also referred to as pillars) that extend along the longitudinal axis L (which may also serve as a longitudinal axis of the pump head 2 and/or pump housing 35) between the proximal ring 26 and a cylindrical section 59 of the shroud 16. In some embodiments, the axially-extending members 60 can be shaped or otherwise be configured to serve as vanes that can shape or direct the flow of blood through the second fluid ports 25. For example, in various embodiments, the axially-extending members 60 can be angled, tapered, or curved (e.g., in a helical pattern) to match the profile of the impeller blades 40 and/or to accelerate blood flow through the pump 2. In other embodiments, the axially-extending members 60 may not be angled to match the blades 40. In some embodiments, the first fluid port 27 can comprise an inlet port into which blood flows. In such embodiments, the impeller assembly 4 can draw blood into the first fluid port 27 and can expel the blood out of the pump 2 through the second fluid ports 25, which can serve as outlet ports. In other embodiments, however, the direction of blood flow may be reversed, in which case the second fluid ports 25 may serve as fluid inlets and the first fluid port 27 may serve as a fluid outlet.

As shown in FIGS. 1A-1D, the system 1 comprises the drive unit 9 with the motor 30 that can be sealed in the motor housing 29. The drive magnet 17 can be rotatable by the motor 30 by way of a motor shaft 51. The motor 30 can electrically connect to the power lead 20. The power lead 20 can serve as a flexible tether that comprises an elongate tension member configured to oppose loads applied in opposite directs at opposite ends of the power lead 20. In one embodiment the power lead 20 is hollow, as discussed further below. As shown in FIGS. 1D and 1F, the power lead 20 can comprise an insulating body having a central lumen 55 and a plurality of (e.g., three) outer lumens 56A-56C extending along a length of the power lead 20. One or more electrical conductors (such as the elongate conductors 73a-73c shown in FIG. 1L) can be disposed in the hollow elongate power lead 20 and can be configured to convey current to the motor 30 from a source, such as the external control system. For example, in some embodiments, the outer lumens 56A-56C can be sized and shaped to receive corresponding electrodes or electrical wires (not shown in FIGS. 1F-1G, but illustrated in the arrangement of FIG. 1L) to provide electrical power to the motor 30. For example, the lumens 56A-56C can receive wires configured to supply ground and drive voltage to corresponding windings on the motor. The electrodes or conductors can extend through corresponding openings 57A-57C of a motor mounting support 54 configured to support the motor 30.

The central lumen 55 can be sized and shaped to receive an elongate stiffening member or guidewire (not shown). In some embodiments, the stiffening member or guidewire can be inserted through a proximal opening 65 at the proximal end 21 (see FIG. 1G) into the central lumen 55 during delivery to help guide the pump 2 to the treatment location or maintain the pump 2 in a given location. In some embodiments, as explained in Section II, the stiffening member can be inserted into the proximal opening 65 and advanced through the central lumen 55 to bear against the pump 2 to facilitate removal of a pump delivery system. The stiffening member or guidewire can be easily inserted and removed when finished. As shown in FIG. 1G, the connector 23 near the proximal end 21 of the system 1 (e.g., at a proximal end portion of the lead 20) can have a plurality of electrical contacts 58A-58C electrically connected to the wires or conductors in the corresponding outer lumens 56A-56C. As shown, the contacts 58A-58C can be disposed on an outer surface of the lead 20. The contacts 58A-58C can comprise rings spaced apart by an insulating material 70 and can be configured to electrically connect to corresponding electrical components in the control system or console (not shown). It should be appreciated that, in some medical devices (e.g., in some percutaneous blood pumps or other types of devices), any of the lumens 55, 56A-56C can be used for other functions, such as, for example, the delivery of fluid to the target location and/or the removal of fluid from the target location. The lumens 55 and/or 56A-56C can be used to provide any suitable type of communication with the target location, including, e.g., one or more of mechanical communication (for example, by way of providing access for a guidewire, stiffening element, lead attachment device, actuating wire for a slip ring (such as the slip ring 131 of FIGS. 13A-13C), actuating wire to act on the struts, etc.), electrical communication (for example, by way of one or more elongate conductor(s)), and/or fluid communication (serving as, for example, a fluid delivery or outlet lumen).

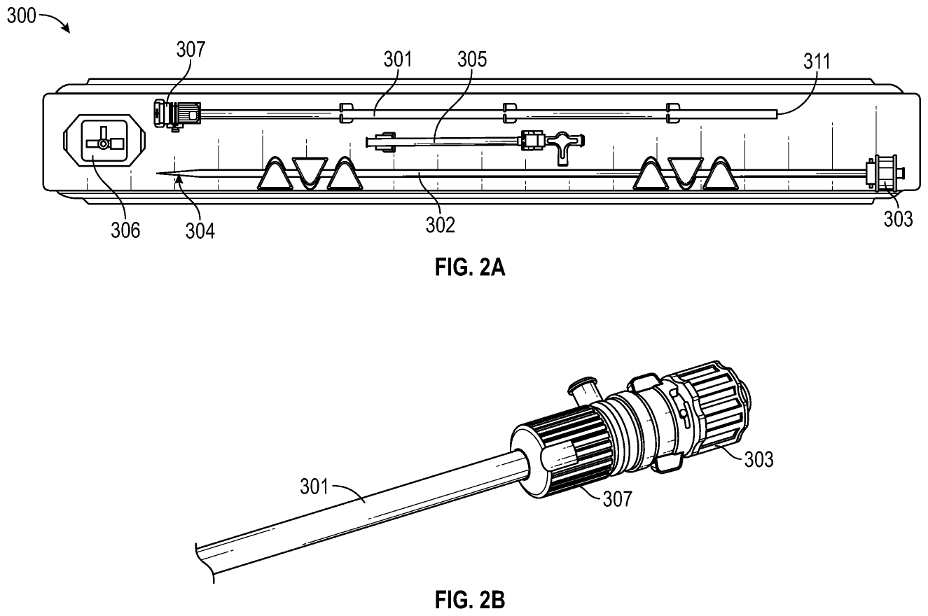

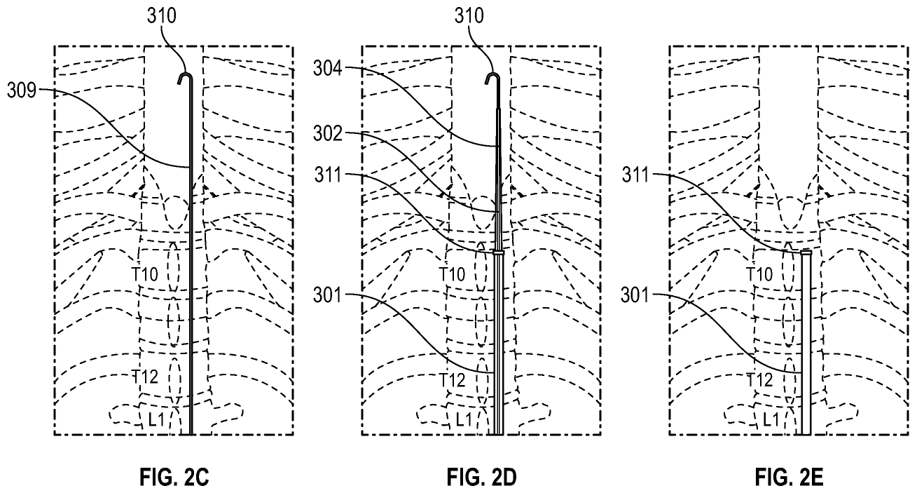

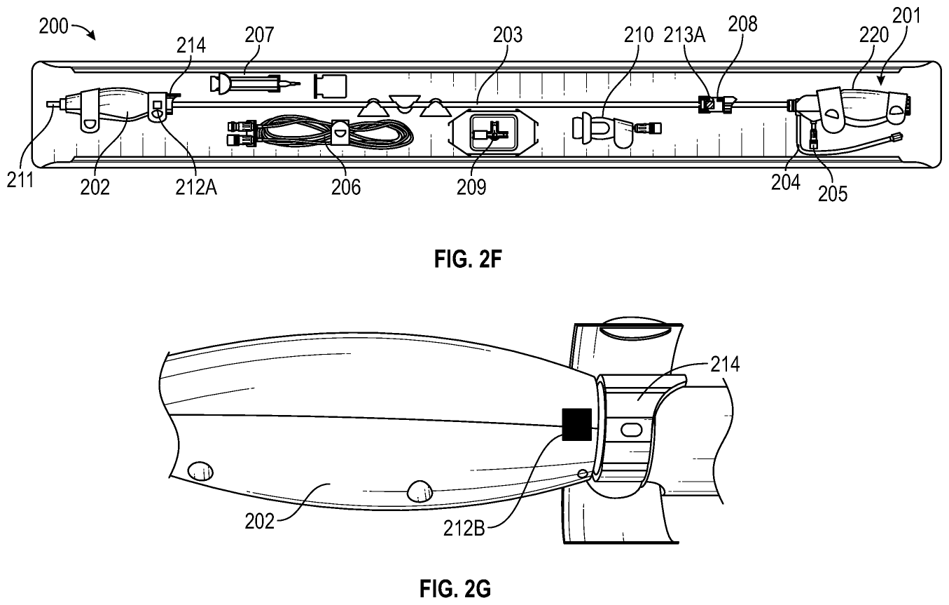

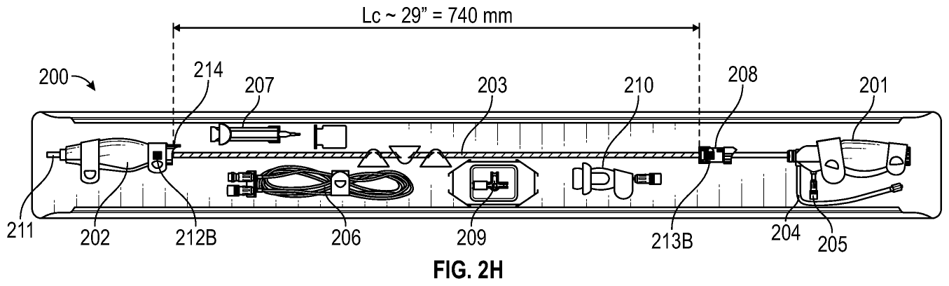

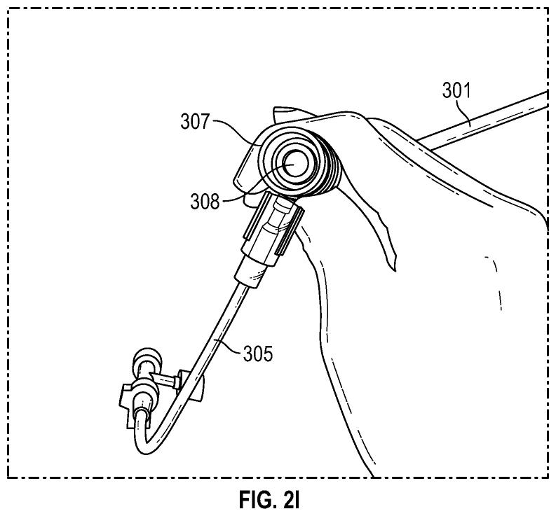

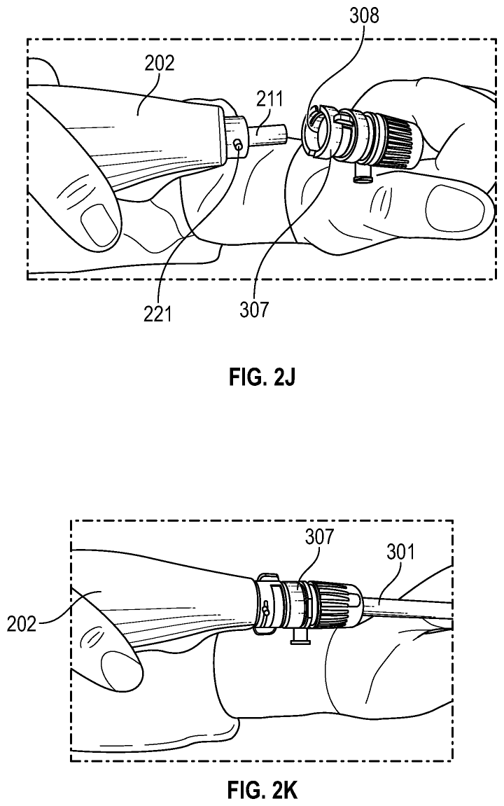

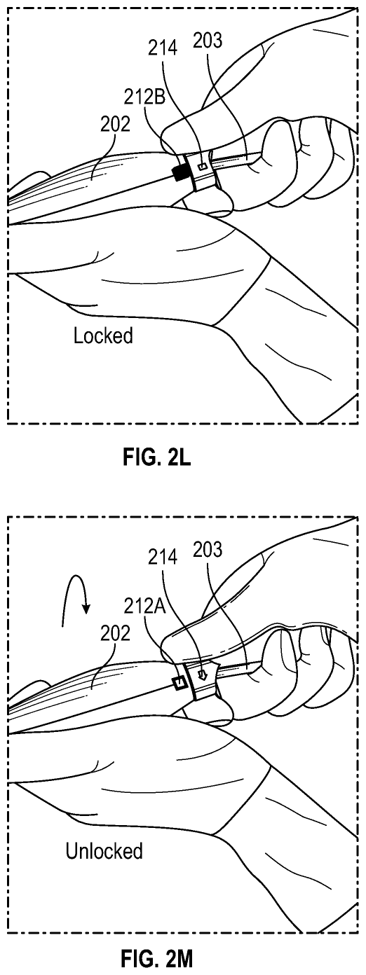

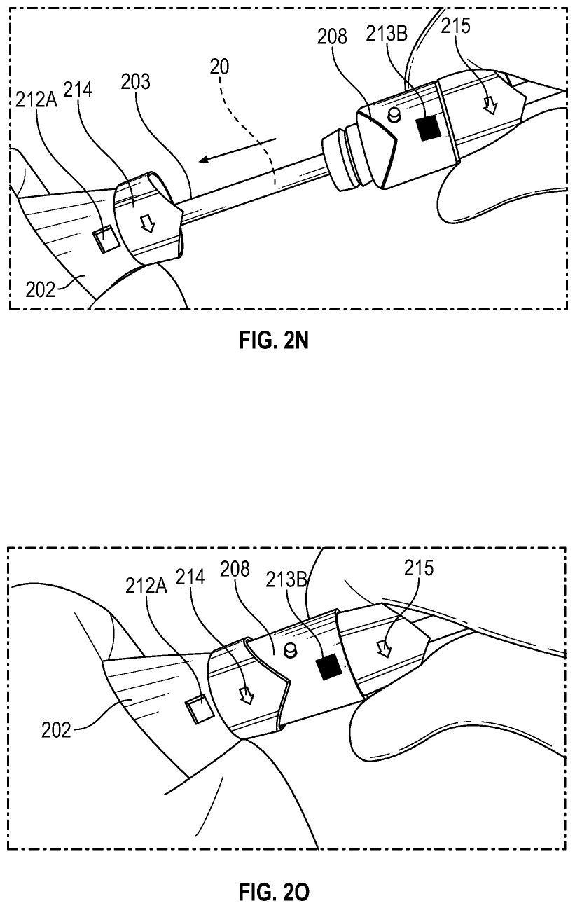

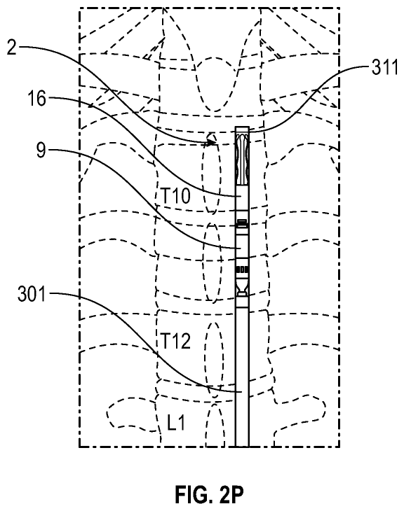

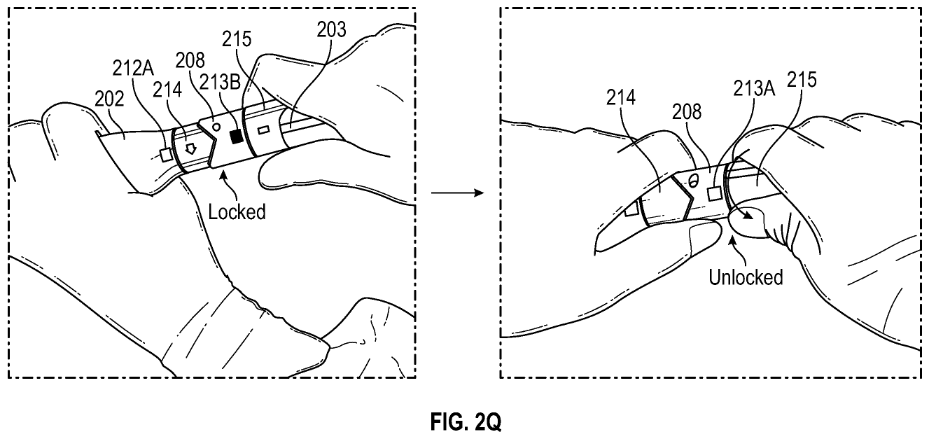

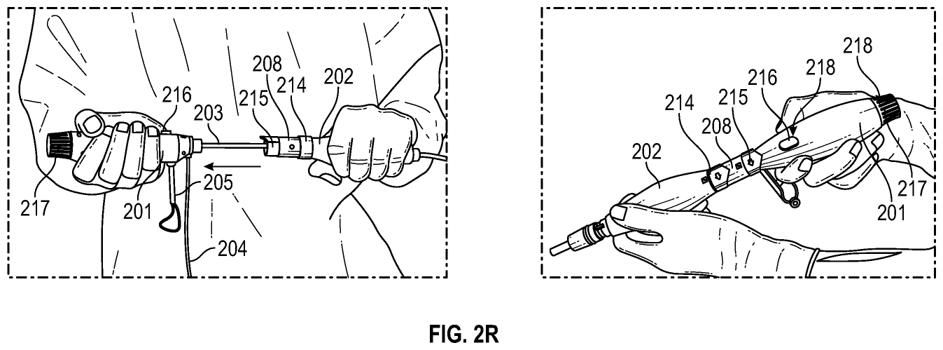

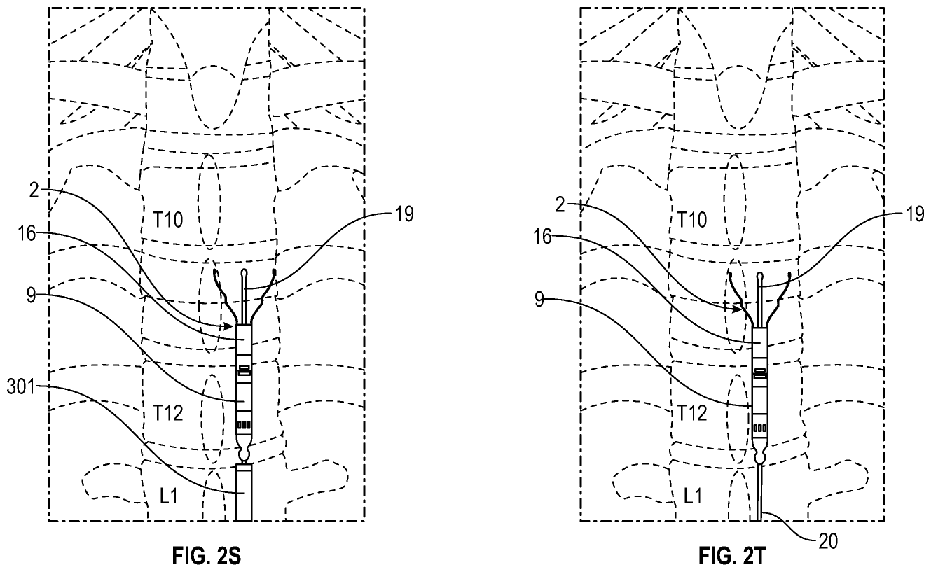

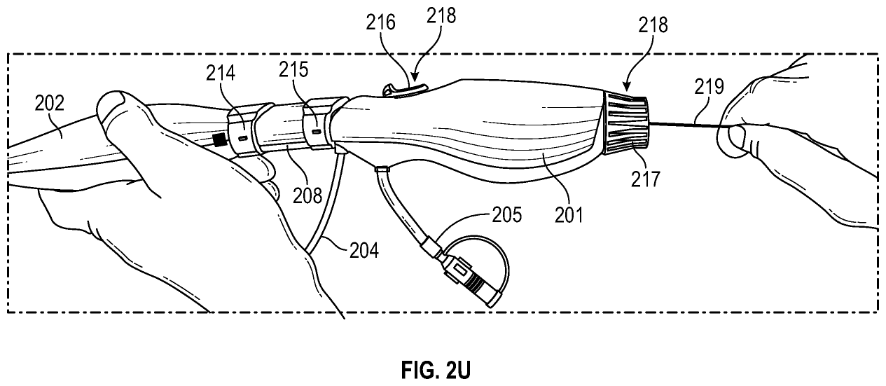

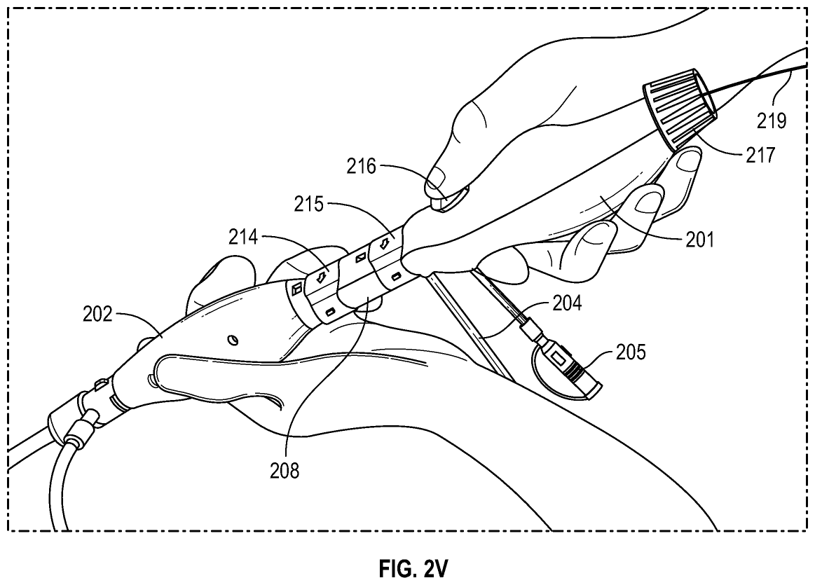

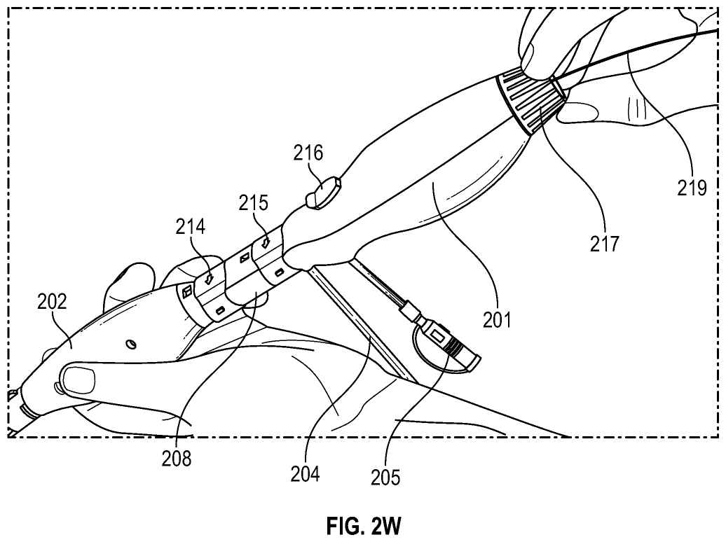

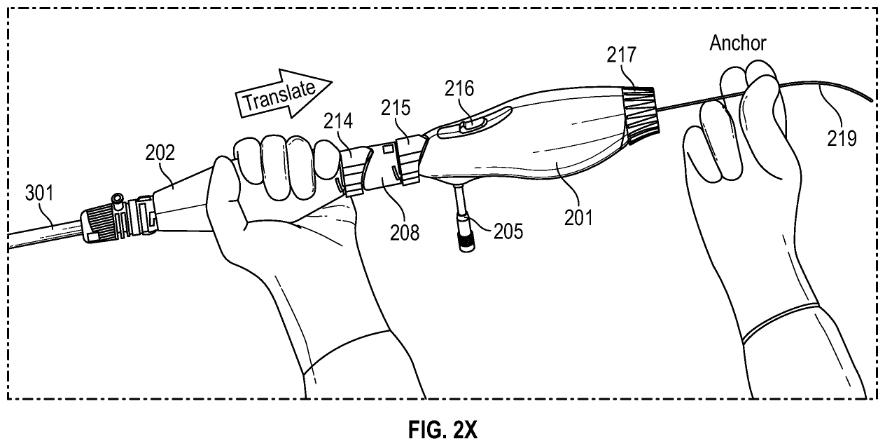

In some embodiments, the lead 20 can have an outer jacket 75 comprising an insulating material that can be the same as or different from the insulating material 70. In various embodiments, the outer jacket 75 can comprise a polymer. In some arrangements, the outer jacket 75 can comprise silicone. However, the use of silicone in the lead 20 may generate excessive frictional forces between the lead 20 and an inner delivery catheter 203 of a delivery system 200 (see FIG. 2F et seq.), which may make delivery and/or retrieval more difficult for the clinician. Accordingly, in various embodiments, the outer jacket 75 can comprise a polyurethane outer surface, e.g., a polyurethane coating. Beneficially, the use of polyurethane for the insulating material of the outer jacket 75 can provide a reduced-friction interface between the power lead 20 and the delivery catheter 203, which can provide an easier delivery and/or retrieval process.

In addition, as shown in FIG. 1G, a transverse opening 68 can extend through a sidewall 69 of the lead 20. As shown in FIG. 1G, the transverse opening 68 can be disposed at the proximal end portion of the lead 20 adjacent the proximal end 21 of the lead 20, e.g., spaced distally from the proximal end 21 by a small distance (e.g., by less than 10 cm, less than 5 cm, less than 1 cm from the proximal end 21). The transverse opening 68 can be disposed between a proximal-most contact 58C and the proximal end 21. The transverse opening 68 can be used during assembly to position the lead 20 within the proximal handle 201 as explained below.

Beneficially, the blood flow assist system 1 can be delivered percutaneously to a treatment location in the patient. FIG. 1H shows the pump 2 disposed within an elongate sheath 28. As shown, the struts 19 are held in a collapsed configuration by the inner wall of the sheath 28. As discussed further below, the struts 19 can be configured to collapse in a controlled manner, e.g., with at least a portion deflected away from inner wall of the sheath 28 when disposed in the sheath. As shown, the struts 19 can comprise knees 102, which can serve to space distal ends of the struts 19 (e.g., at or near the contact pads 24 or hooks) from the inner wall of the sheath 28, such that there is a space 46 between the contact pads 24 or hooks and the inner wall of the sheath 28 in the collapsed configuration within the sheath 28.

The knees 102 can be of the same configuration for each of the struts 19 in one embodiment. In such an embodiment, the struts 19 may all collapse or fold in the same manner within the sheath 28. In another embodiment the knee 102 of one or more struts 19 can be differentiated from the knee 102 of one or more other struts 19 such that the struts are collapsed or folded in different manners. As explained herein, in various embodiments, the struts can be longitudinally-aligned or longitudinally-offset or staggered. For example, a pair of opposing struts 19 (e.g., disposed radially opposite one another) can have knees 102 that cause the opposing strut of the pair to collapse prior to the collapsing of other struts 19 of the pump 2. In one example, the pump 2 has four struts 19. Two opposing struts 19 are configured to bend at the knees 102 prior to the bending of the knees of the other struts 19. As such, the two opposing struts 19 can be collapsed to a position between the other two struts to provide a compact arrangement. The knees 102 can be configured such that some struts undergo a greater degree of bending or collapsing. Thus the space 46 between the contact pads 26 and the inner wall of the sheath 28 can be two to six (and in some cases three to four) times greater for one or more, e.g., a pair of, struts than for one or more, e.g., another pair of struts 19, which can be provided to avoid tangling of the struts. Accordingly, in various embodiments, some struts may be structured to collapse first when engaged with the sheath 28, and the remaining struts can collapse as the sheath 28 induces the collapsing of the initial struts.

In some embodiments, one or more struts comprises knees 102 that can control the order of collapsing of the struts. For example one or more struts can have a knee 102 positioned more proximally compared to the position of the knees 102 of one or more other struts. In one example, two opposing struts 19 can have knees 102 disposed more proximally than are the knees 102 of another strut 19. In one example, a first set of opposing struts 19 have knees 102 disposed more proximally than a second set of struts 19 disposed approximately 90 degrees offset from the first set of struts 19. This can allow the first set of struts to be more completely folded by distal advancement of the sheath 28 before a more complete folding of the second set of struts 19. In a further variation, knees 102 can be longitudinally spaced apart on adjacent struts 19 so that adjacent struts fold at different times or rates. The illustrated embodiments includes the knees 102, but in other embodiments, no knees may be provided. For example, the struts 19 can be retracted at different rates by hinges and/or by modifying material thickness or properties in or along the length of one or more struts 19 to control the timing or rate of folding upon advancing the sheath 28. A living hinge structure can be formed along the length of one or more struts 19 to control timing, rate, and/or sequence of retraction of the struts 19. In one example, an area of reduced thickness transverse to the length of a strut 19 causes the strut to fold or bend when a sheath is advanced across the reduced thickness area. By offsetting the longitudinal position of reduced thickness areas in the struts 19, the sequence of retraction can be controlled.

In the collapsed configuration, the struts 19 can be compressed to a diameter or major lateral dimension at one or more locations that is approximately the same as (or slightly smaller than) the diameter of the shroud 16. Thus, as shown in the collapsed configuration of FIG. 1H, at least a portion of the struts 19 are compressed to a diameter or major lateral dimension that is smaller than the major lateral dimension or diameter of the pump housing 35, shroud 16 and/or the drive unit 9. In some embodiments, at least a portion of the struts has a major lateral dimension that is no more than a major lateral dimension of the pump housing 35. In some embodiments, at least a portion of the struts has a major lateral dimension that is less than a major lateral dimension of the pump housing 35 and/or the motor housing 29. The patient can be prepared for the procedure in a catheterization lab in a standard fashion, and the femoral artery can be accessed percutaneously or by a surgical approach. The sheath 28 (or a dilator structure within the sheath 28) can be passed over a guidewire and placed into the treatment location, for example, in the descending aorta. After the sheath 28 is placed (and the dilator removed), the pump 2 can be advanced into the sheath 28, with the pump 2 disposed in the mid-thoracic aorta, approximately 4 cm below the take-off of the left subclavian artery. In other embodiments, the pump 2 and sheath 28 can be advanced together to the treatment location. Positioning the pump 2 at this location can beneficially enable sufficient cardiac support as well as increased perfusion of other organs such as the kidneys. Once at the treatment location, relative motion can be provided between the sheath 28 and the pump 2 (e.g., the sheath 28 can be retracted relative to the pump 2, or the pump 2 can be advanced out of the sheath 28). The struts 19 of the localization system can self-expand radially outwardly along the radial axis R due to stored strain energy into the deployed and expanded configuration shown in FIGS. 1A-IC. In some embodiments, such as those in which the vasculature is accessed by the femoral artery, the struts 19 can extend distally, e.g., distally beyond a distal end of the shroud 16 and/or the impeller 6. In other embodiments, as explained herein, the pump 2 can be delivered percutaneously through a subclavian artery. In such embodiments, the struts 19 may extend proximally, e.g., proximal the pump housing 35 and/or the motor housing 29. In still other embodiments, multiple pluralities of struts may extend proximally and distally relative to the pump 2. The convex contact pads 24 can engage the blood vessel wall to stabilize (e.g., assist in anchoring) the pump 2 in the patient's vascular system. Once at the treatment location, the clinician can engage the control system to activate the motor 30 to rotate the impeller assembly 4 to pump blood.

Thus, in some embodiments, the pump 2 can be inserted into the femoral artery and advanced to the desired treatment location in the descending aorta. In such arrangements, the pump 2 can be positioned such that the distal end 22 is upstream of the impeller 6, e.g., such that the distally-located first fluid port 27 is upstream of the second fluid port(s) 25. In embodiments that access the treatment location surgically or percutaneously via the femoral artery, for example, the first fluid port 27 can serve as the inlet to the pump 2, and the second ports 25 can serve as the outlet(s) of the pump 2. The struts 19 can extend distally beyond a distal end of the pump housing 35. In other embodiments, however, the pump 2 can be inserted percutaneously through the left subclavian artery and advanced to the desired treatment location in the descending aorta. In such arrangements, the pump 2 can be positioned such that the distal end 22 of the system 1 is downstream of the impeller 6, e.g., such that the distally-located first fluid port 27 is downstream of the second fluid port(s) 25. In embodiments that access the treatment location through the left subclavian artery, the second fluid port(s) 25 can serve as the inlet(s) to the pump 2, and the first port 27 can serve as the outlet of the pump 2.

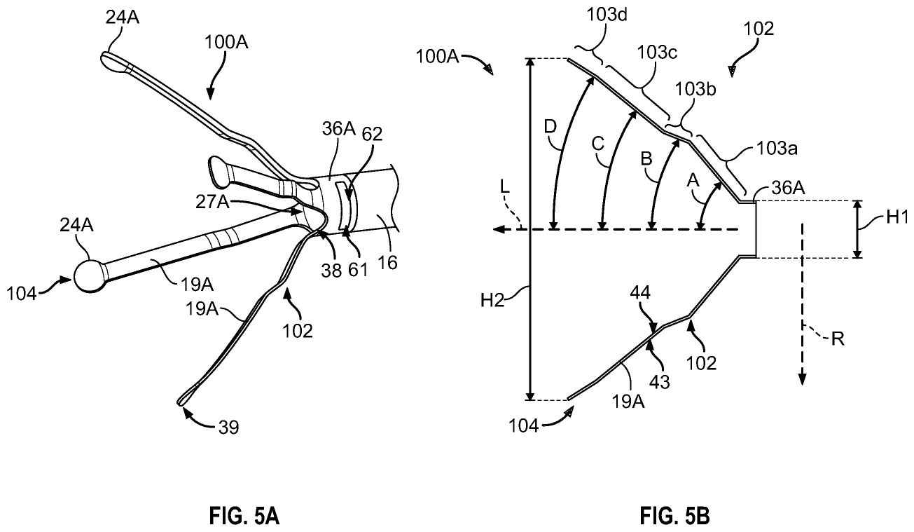

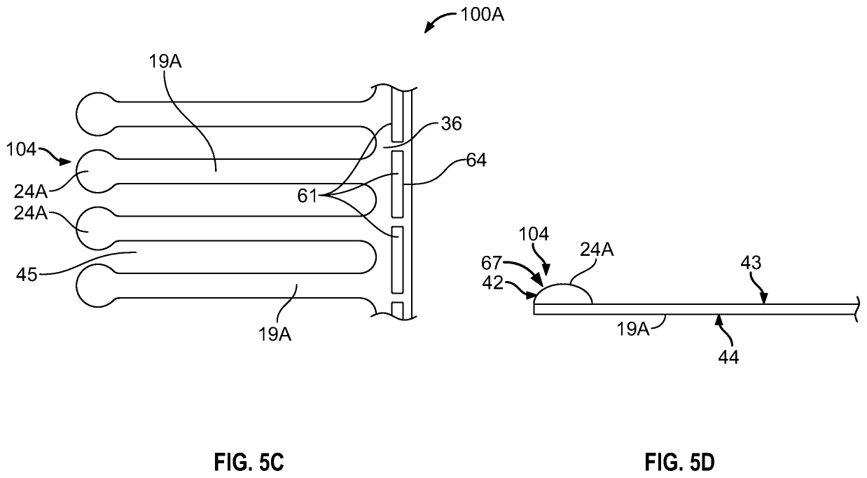

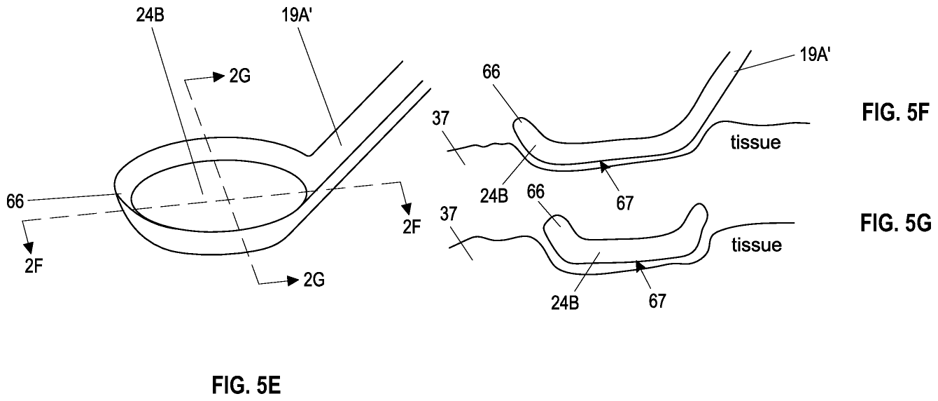

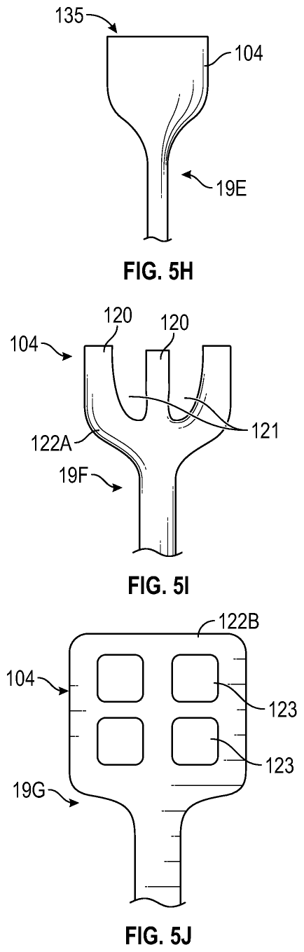

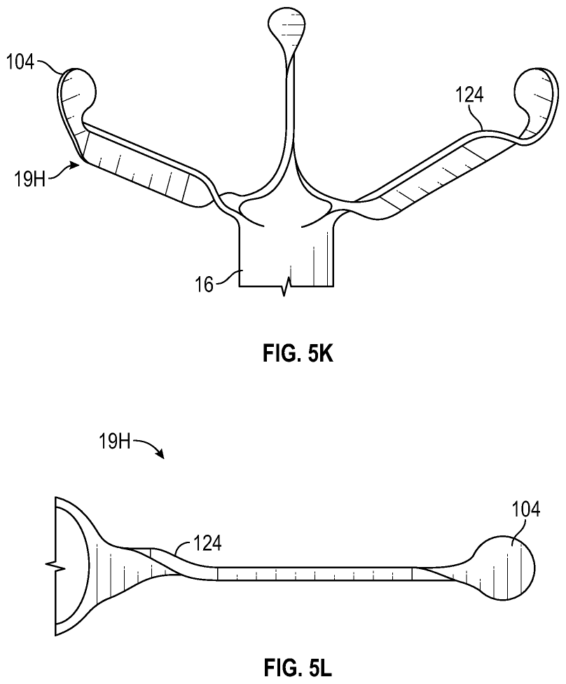

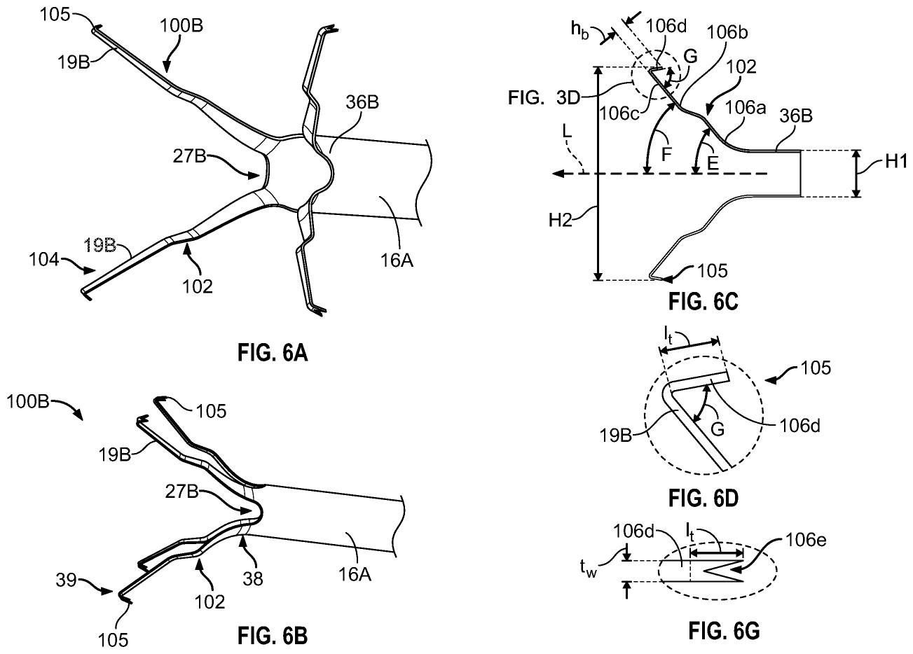

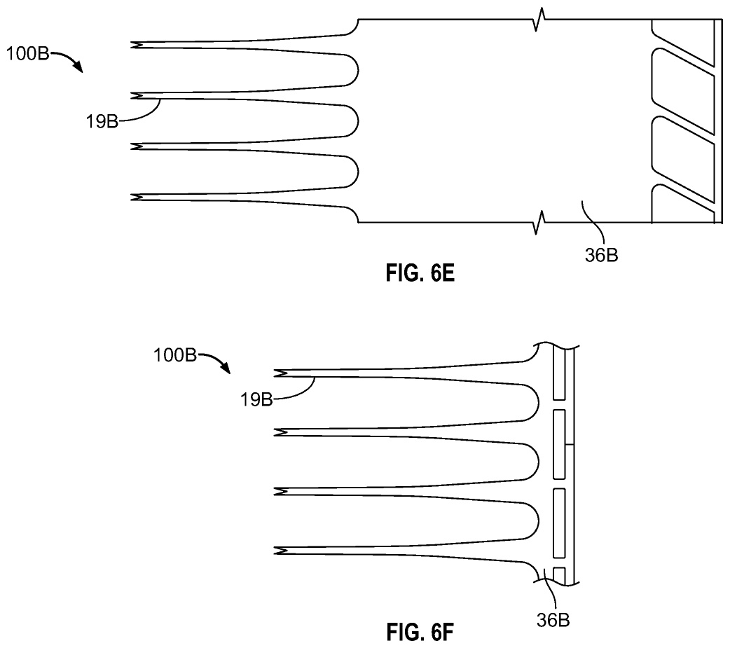

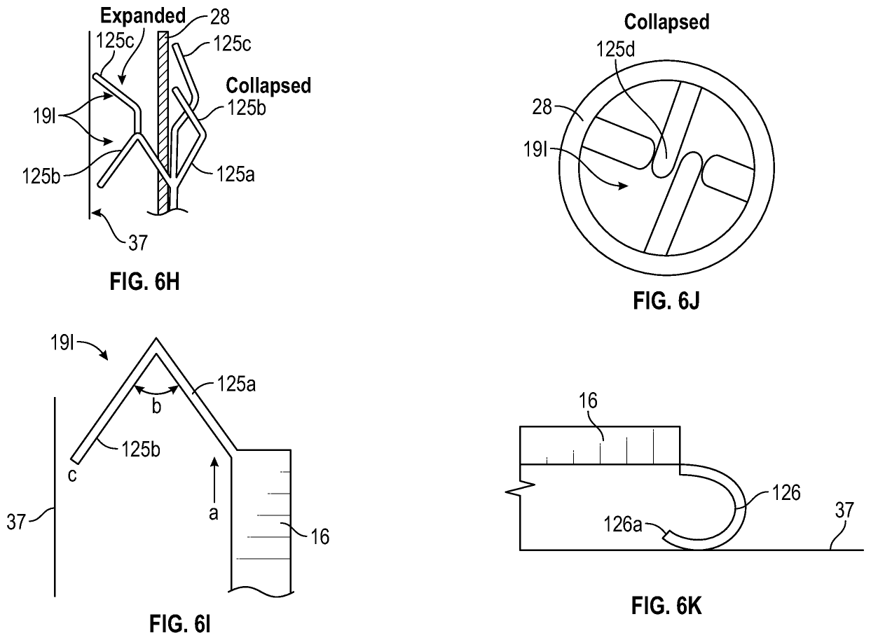

When the treatment procedure is complete, the pump 2 can be removed from the patient. For example, in some embodiments, the pump can be withdrawn proximally (and/or the sheath 28 can be advanced distally) such that a distal edge of the sheath 28 engages with a radially-outer facing surface 43 of the struts 19. In some embodiments, the distal edge of the sheath 28 can engage with the knees 102 of the struts (see, e.g., FIGS. 5A-6C). The distal edge of the sheath 28 can impart radially-inward forces to the radially-outer facing surface 43 (e.g., at approximately the location of the knees 102) to cause the struts 19 to collapse and be drawn inside the sheath 28. Relative motion opposite to that used for deploying the pump 2 can be provided between the sheath 28 and the pump 2 (e.g., between the sheath 28 and the impeller assembly 4 and pump housing 35) to collapse the struts 19 into the sheath 28 in the collapsed configuration. In some embodiments, the pump 2 can be withdrawn from the sheath 28 with the sheath 28 in the patient's body, and the sheath 28 can be subsequently used for another procedure or removed. In other embodiments, the sheath 28 and the pump 2 can be removed together from the patient's body.

The foregoing description includes embodiments in which a proximal end of a drive shaft 51 is located in the drive unit 9. The proximal end of the drive shaft 51 and the motor 30 are disposed within the body in use. FIG. 1J shows another embodiment in which a motor 30A is disposed outside the body in use. An elongate, flexible shaft 51′ is coupled at a distal end with the drive magnet 17. The shaft 51′ extends through an elongate body 20′ and is or can be coupled at a proximal end thereof with a motor 30A. The motor 30A can be larger than the motor 30 since it need not be disposed within the profile of the sheath 28. The elongate body 20′ may have one or more lumens. The shaft 51′ may extend through the central lumen 55. One or more outer lumens 56a may be provided to flow a fluid into the system to lubricate and/or cool the shaft 51′. Rotation of the proximal end of the shaft 51′ by the motor 30a results in rotation of the entire length of the shaft 51′ through the elongate body 20′ and also results in rotation of the drive magnet 17. Rotation of the drive magnet 17 causes rotation of one or more magnets in the impeller 6 to create flow through the pump 2 by virtue of magnetic attraction of these magnets across the distal drive unit cover. In other embodiments, the shaft 51′ can be directly mechanically coupled to the impeller 6 such that rotation does not depend on magnetic coupling. One or more shaft rotation supports 54A can be provided within a distal housing 29A to support a distal portion of the shaft 51′. The elongate body 20′ and/or the shaft 51′ can comprise a tether to control or to aid in control of the position of the pump, e.g., to counter thrust forces of the impeller 6 to reduce or minimize movement of the pump 2 in operation.

Additional details of the pump 2 and related components shown in FIGS. 1A-1H may be found throughout International Patent Application No. PCT/US2020/062928, filed on Dec. 2, 2020, and in U.S. Pat. No. 11,324,940, the entire contents of each of which are incorporated by reference herein in their entirety and for all purposes.

FIGS. 1K-IN illustrate various portions of a power lead 20A according to some embodiments. Unless otherwise noted, components in FIGS. 1K-IN may be generally similar to or the same as like-numbered components of FIGS. 1F-1G. As explained in Section II, a locking pin 245 of a proximal handle 201 (see FIGS. 2F and 31) can be provided to releasably connect the lead 20A (or lead 20) to the proximal handle 201. The locking pin 245 can be releasably inserted into a recess to prevent longitudinal movement of the power lead 20A when the proximal handle 201 is in a locked configuration. In the embodiment shown in FIGS. 1K, 1M, and 1N, the recess can comprise a groove 71 disposed near the proximal end of the lead 20A. The groove 71 can extend into the proximal end portion of the lead 20A. The groove 71 can be disposed between the proximal end 21 and the contacts 58A-58C. In other embodiments, the recess that engages with the locking pin 245 can comprise the transverse opening 68 which extends into the proximal end portion of the lead 20A, such that the locking pin 245 can removably insert into the transverse opening 68. In various embodiments, the groove 71 can extend partially into a thickness of a sidewall of the lead 20A. The groove 71 can comprise an at least partially annular structure that is disposed at least partially around (e.g., completely around) a perimeter or circumference of the lead 20A. In some embodiments, the groove 71 can be disposed only partially about the perimeter of the lead 20A. In some embodiments, the recess can comprise a notch extending into the lead 20A. The recess can be disposed at a distance of less than 20 cm, less than 10 cm, or less than 5 cm from the proximal end 21 of the lead 20A. In various embodiments, a thickness of the elongate lead 20A may not be uniform along a length of the elongate lead 20A. For example, the recess can comprise a necked portion comprising a reduced diameter of the lead 20A. It should be appreciated that, in other embodiments, the lead 20 or 20A can comprise a locking pin or projection and the proximal handle 201 can comprise a recess or groove. Other mechanisms to connect the proximal handle 201 and the lead 20 or 20A may be suitable.

As shown in FIG. 1L, the elongate conductors 73a-73c can extend through respective outer lumens 56A-56C. The elongate conductors 73a-73c may not be positively attached to the main body of the lead 20A (e.g., at an intermediate span of the lead) except for at the contacts 58A-58C, such that the conductors 73a-73c may slide within the lead 20, which can increase flexibility of the lead 20A. Further, the presence of the conductors 73a-73c can serve to stiffen the lead, which can assist in advancing the lead 20 within the vasculature without bunching up. The presence of the conductors 73a-73c can also serve to strengthen the lead, so that it is capable of withstanding greater tensile loads. Thus, the conductors 73a-73c can provide large-scale flexibility so as to enable the lead 20 to bend casily, while also restricting tight bends or kinks in the lead 20.

In FIG. 1N, in some embodiments, an engagement feature 72 can be provided at the proximal end 21 of the lead 20A. In other embodiments, the engagement feature 72 can be provided as threads on an outer diameter of the proximal end of the lead 20A. The engagement feature 72 may include threads or other mechanical connection that can enable the clinician to capture the power lead 20A if desired during a procedure. For example, the clinician can insert a retrieval device into the proximal handle 201 (or into whatever lumen provides access to the lead 20A). The retrieval device can have a corresponding engagement feature (e.g., threads or other mechanical connection) that can engage with the engagement feature 72 of the lead 20A to provide a positive connection therebetween. The clinician can remove the lead 20A, or otherwise manipulate the position of the lead 20A, by moving the retrieval device. In various embodiments, the engagement feature 72 can be configured to couple to a lead attachment device 407 for applying tension to the lead 20A, e.g., during removal.

### II. Example Introducer and Delivery Systems

FIG. 1O is a schematic diagram showing a percutaneous medical system 500 including an elongate tether 501 having a connector 503 at a proximal end portion of the tether 501. A percutaneous medical device 502 can be coupled to a distal end portion of the tether 501. In some embodiments, the medical device 502 can have a deployment diameter D that is larger than a diameter of the tether 501. In other embodiments, the diameter D can be the same as or less than the diameter of the tether 501. The medical device 502 can be advanced into a patient's body to a target treatment location and can be deployed and/or activated to treat the patient. The elongate tether 501 can comprise any suitable type of elongate member. For example, in various embodiments, the elongate tether 501 can serve to provide electrical communication and/or a mechanical connection between the medical device 502 and an external control system. In some embodiments, the elongate tether 501 can serve to provide only electrical communication between the medical device 502 and the external control system (e.g., the tether 501 may not provide a mechanical connection between the medical device 502 and the control system). In some embodiments, the elongate tether can serve to provide only mechanical connection between the medical device 502 and the external control system (e.g., the tether 501 may not provide an electrical connection between the medical device 502 and the control system). In some embodiments, such as those described herein in connection with the pump 2, the elongate tether 501 can provide both electrical communication and a mechanical connection between the medical device 502 and the external control system.

It can be challenging to deploy the percutaneous medical device 502 into a body cavity or body lumen of a patient, particularly in medical devices 502 that include the elongate tether 501 extending from within the body cavity or body lumen to the outside of the patient's body. In the illustrated embodiments, the percutaneous medical system 500 comprises a blood flow assist system 1, and the medical device 502 comprises an intravascular blood pump 2 connected to an elongate tether 501 comprising a power lead 20, 20A. However, it should be appreciated that the introducer system, the delivery system, and/or the retrieval system disclosed herein can be used with any suitable type of percutaneous medical device 502 that includes an elongate tether 501. For example, the medical device 502 can comprise an intracardiac blood pump (e.g., a catheter-based left ventricular assist devices, or LVAD), which may also be deployed using any of the introducer, delivery, and/or retrieval systems disclosed herein. In addition, other types of percutaneous medical devices 502 may benefit from the introducer, delivery, and/or retrieval systems disclosed herein. For example, medical devices such as stents, stent grafts, inferior vena cava (IVC) filters, pressure or flow sensors, neurostimulation devices, embolic protection devices, or occlusion or flow restricting devices may be deployed or retrieved by the apparatus and means described herein and may include such an elongate tether 501 for permanent (e.g., long-term) or temporary use. In addition, the illustrated embodiments are directed to pumping blood within a blood vessel (e.g., a descending aorta) of the patient. However, it should be appreciated that any of the introducer, delivery, and/or retrieval systems can be used to access other target locations of the anatomy, including other body cavities and/or body lumens. Accordingly, it should be appreciated that, throughout the present disclosure, various disclosed features and systems may be used with any suitable percutaneous medical system 500.

In various embodiments, the tether 501 can comprise one or multiple channels to serve as a pass through for mechanical or electrical elements from proximal to distal without exceeding a deployment diameter. For example, the tether 501 can comprise channels (e.g., outer lumens) to receive electrical conductors to transfer power or signals between the distal end of the pump (e.g., from sensors) to the tether 501 and connector 503. In some embodiments, the tether 501 can comprise channel(s) for mechanical tension or compression elements (e.g., wires or rods) to transfer force from outside the tether 501 to the proximal end of the pump, for example, to provide force to move (e.g., expand or collapse) elements of the device (e.g., struts as described herein or other similar support elements).

FIG. 2A illustrates an introducer set 300, according to various embodiments. The introducer set 300 can be configured to provide percutaneous access to a blood vessel, e.g., the descending aorta. The introducer set 300 can include a delivery sheath 301 having a distal end 311 and a delivery sheath hub 307 at a proximal end of the delivery sheath. The delivery sheath 301 can have a delivery sheath lumen 308 (see FIGS. 21-2J) through which the blood pump 2 can be delivered to a target location in the anatomy. The introducer set 300 can further include an introducer dilator 302 having a distal portion 304 and a dilator hub 303 at a proximal end of the dilator 302. The introducer dilator 302 can comprise a generally cylindrical body that tapers distally at the distal portion 304. This tapered portion allows the effective diameter of the dilator (where it enters the blood vessel) to smoothly vary from a minimum to the diameter of the delivery sheath, thereby facilitating insertion of the delivery sheath. The introducer dilator 304 can assist in maintaining the shape of the delivery sheath 301 and/or the blood vessel during introduction of the delivery sheath 301.

FIG. 2B illustrates the proximal portion of the delivery sheath 301. The introducer dilator 302 can be inserted within delivery sheath lumen 308 of the delivery sheath 301. At least part of the distal portion 304 of the dilator 302 can extend distally past the distal end 311 of the delivery sheath 301 during use. As shown, the dilator hub 303 can be configured to removably connect to the delivery sheath hub 307 of the delivery sheath 301. As shown in FIG. 2B, the dilator hub 303 can be disposed proximal the delivery sheath hub 307. The delivery sheath hub 307 can include a hemostatic valve. In various embodiments, the delivery sheath hub 307 and/or the dilator hub 303 include a port for flushing (e.g., luer extension 305 and stopcock 306) and a swivel to allow relative rotation between the delivery sheath 301 and the dilator 302. The swivel can be provided on the sheath hub 307 and can be configured to allow relative rotation between the sheath 301 and the distal handle 302.

The introducer set 300 can also include a luer extension 305 and a stopcock 306 (see FIG. 2A). The luer extension 305 and the stopcock 306 can connect to the delivery sheath 301 (e.g., to the delivery sheath hub 307) to deliver fluid within the delivery sheath lumen 308. For example, before use, the clinician can connect the luer extension 305 to the delivery sheath hub 307 and can connect the stopcock 306 to the luer extension 305. The inner lumen 308 can be flushed with a fluid (e.g., heparinized saline) to expel air and/or prevent fluids (e.g., blood) from entering the interior of the delivery sheath 301. The introducer dilator 302 can be wetted with saline and inserted into the delivery sheath lumen 308. The delivery sheath hub 307 can be connected or locked to the dilator hub 303 with a quick-connection mechanism (e.g., a bayonet-type connection), for example, by providing relative rotation between the delivery sheath hub 307 and the dilator hub 303. In some embodiments, the dilator 302 can be flushed with saline through a luer connection on a proximal side of the dilator hub 303.

The clinician can access the vasculature using techniques known in the art, e.g., the Seldinger technique. The clinician can insert a guidewire 309 into the patient's vasculature (e.g., the femoral artery) and advance the guidewire 309 to the target treatment location (e.g., into the descending aorta of the patient). FIG. 2C is an image showing the guidewire 309 after insertion into a descending aorta of a patient. In some embodiments, the guidewire 309 may have an atraumatic tip 310, such as a pigtail or J-shaped tip. Turning to FIG. 2D, the delivery sheath 301 and the dilator 302 can be advanced together over the guidewire 309 to a location proximal the atraumatic tip 310. The distal end 311 of the delivery sheath 301 can be positioned approximately at a superior aspect of a T10 vertebral body, as shown in FIGS. 2D and 2E, which can be confirmed by visualizing during fluoroscopy a radiopaque marker at the distal end 311 of the delivery sheath 301. Once the delivery sheath 301 is disposed as shown in FIG. 2D, the dilator 302 and guidewire 309 can be removed from the delivery sheath 301 such that the delivery sheath is disposed in the anatomy as shown in FIG. 2E.

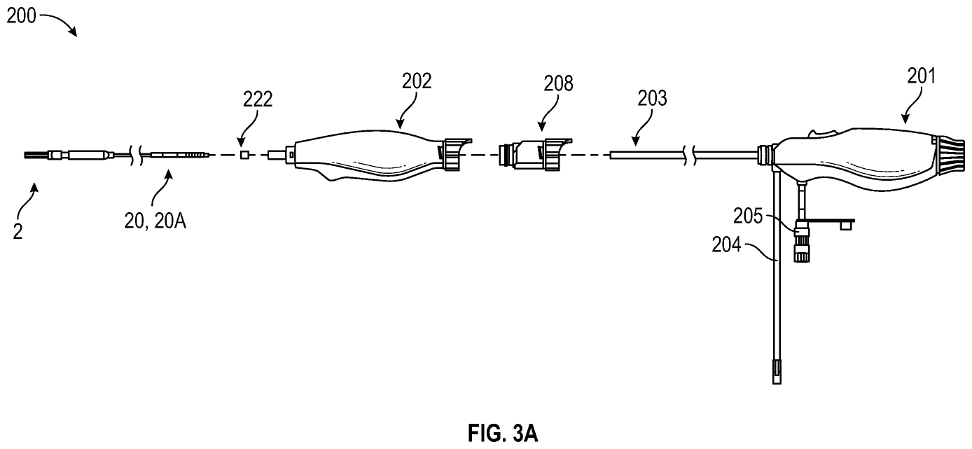

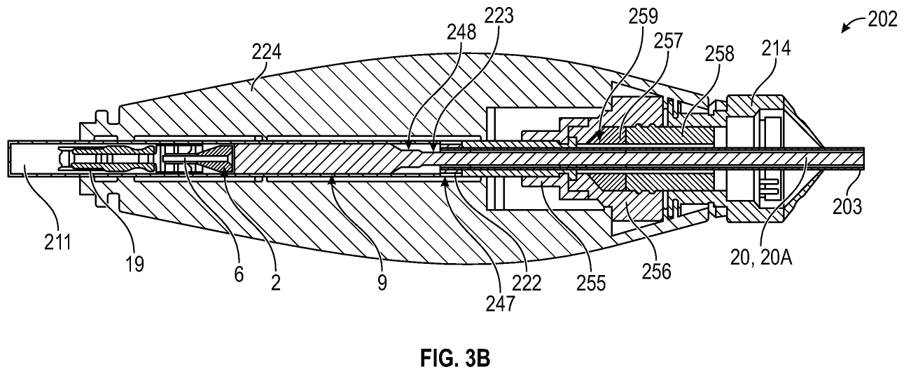

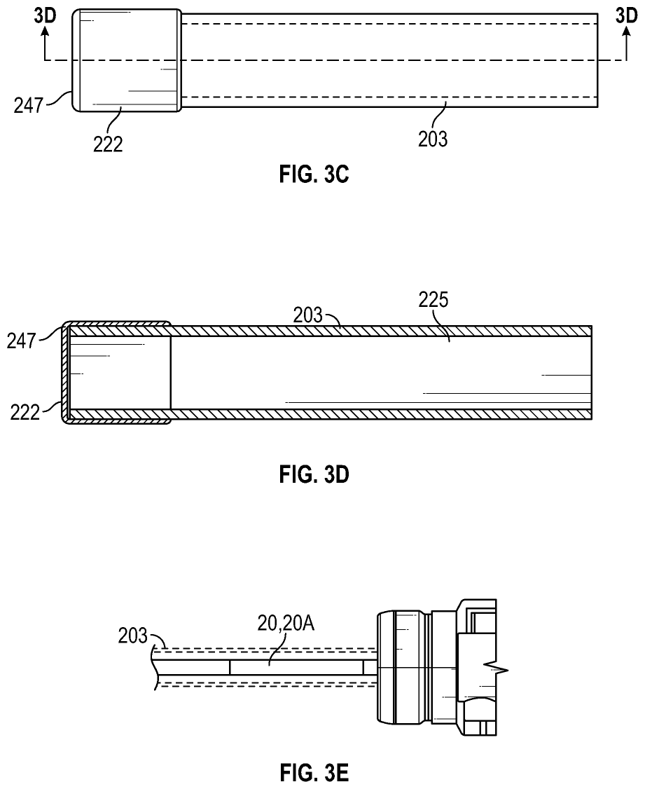

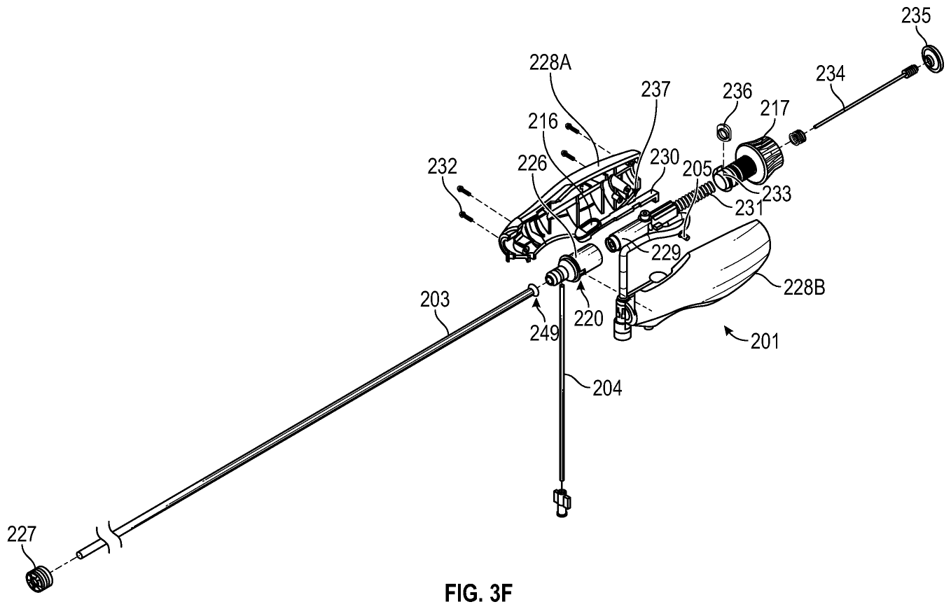

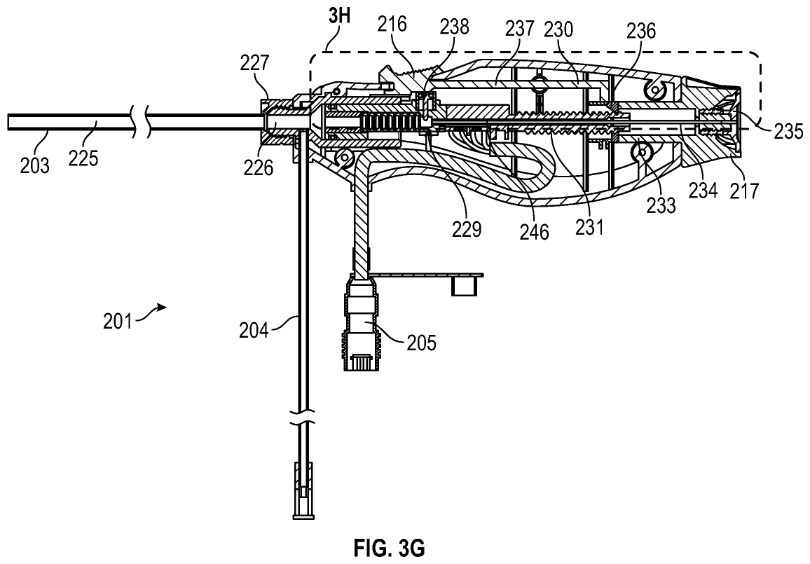

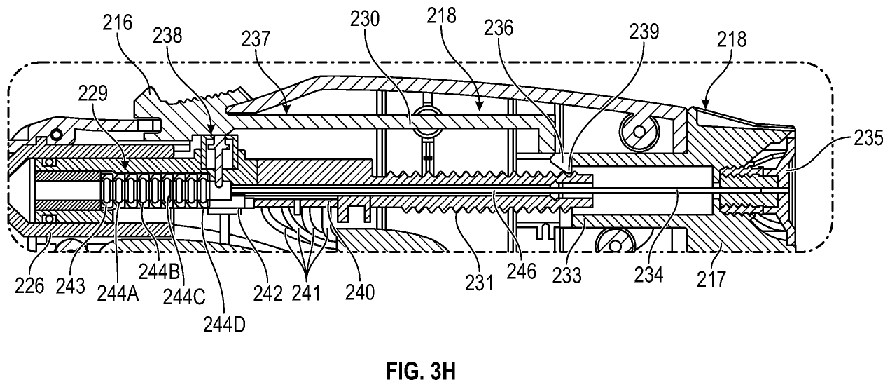

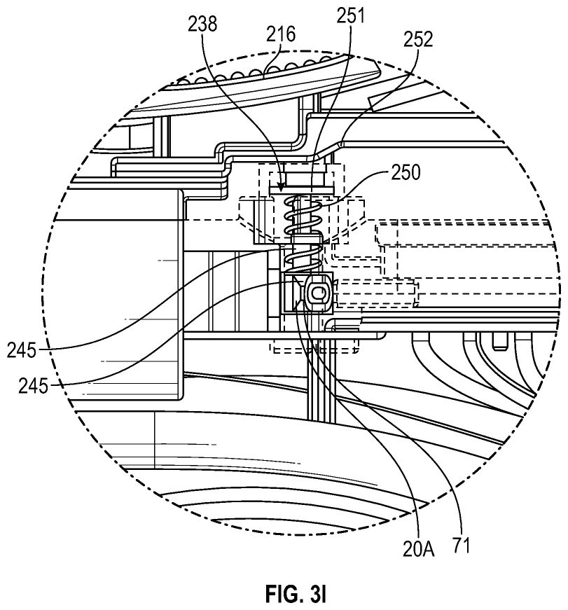

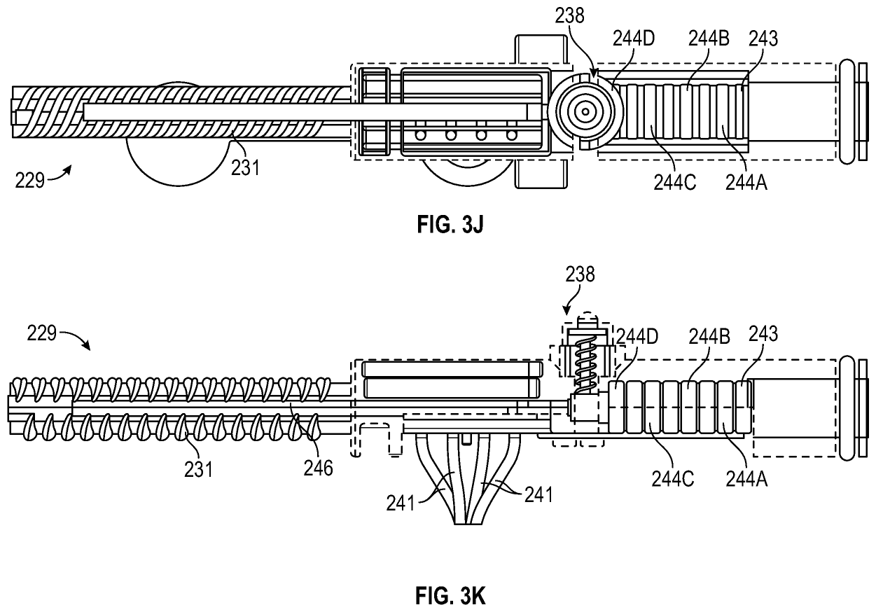

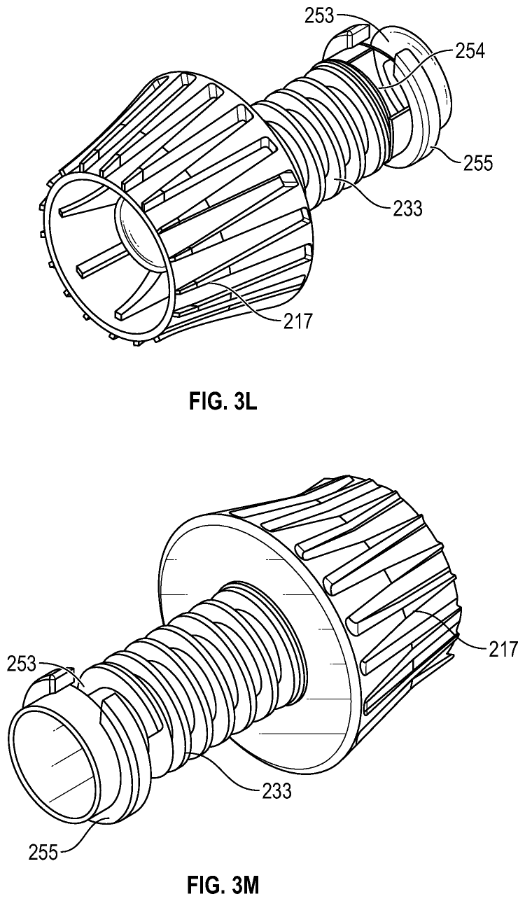

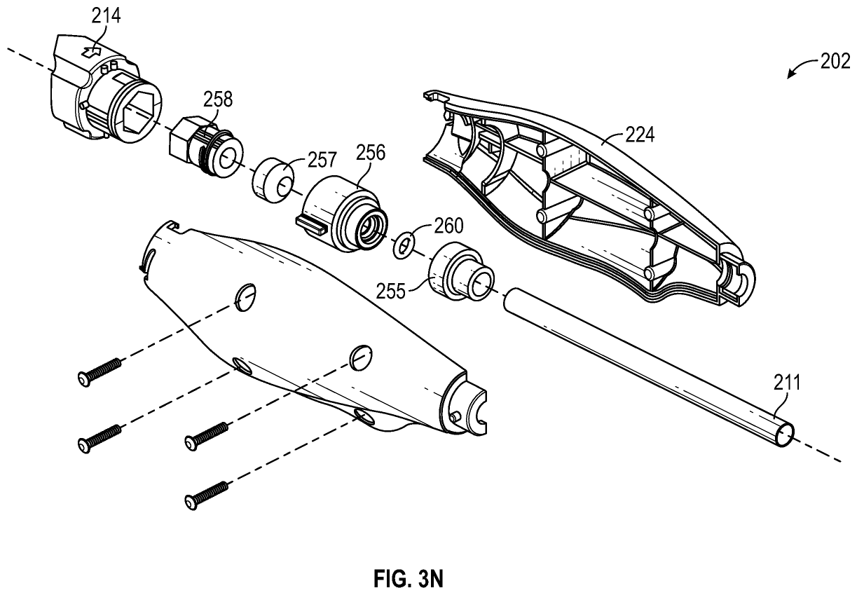

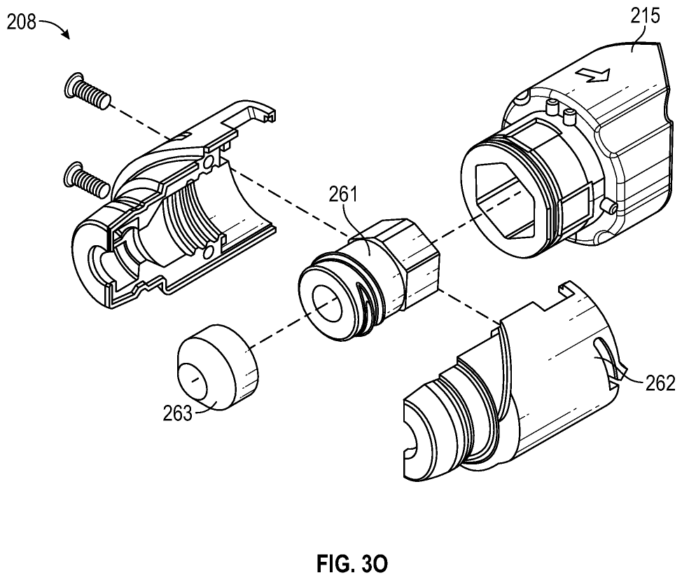

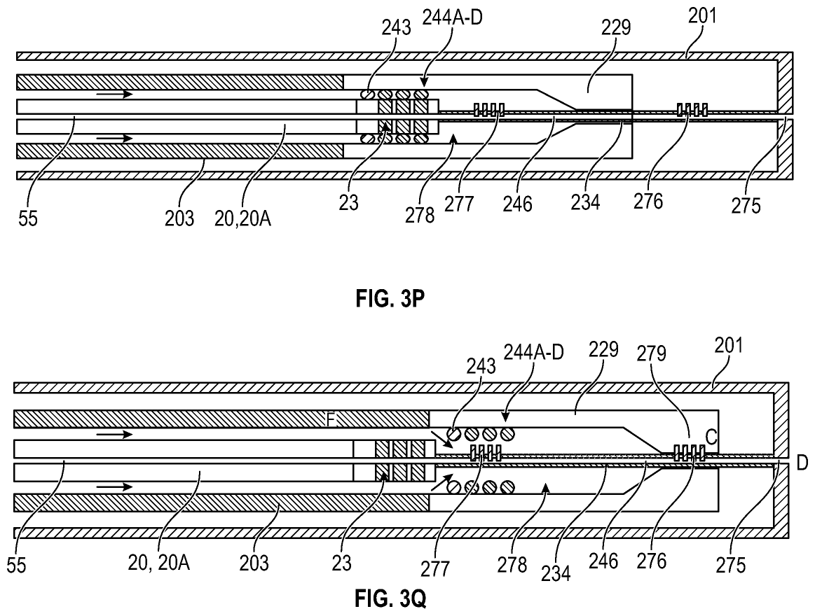

FIG. 2F illustrates a delivery system 200 for a percutaneous medical device, e.g., an intravascular blood pump 2, in an unlocked configuration, according to various embodiments. The delivery system 200 can include a distal handle 202, a proximal handle 201 disposed proximal of the distal handle 202, and delivery catheter 203 extending distally from a distal portion of a housing body 220 of the proximal handle 201. In some embodiments, a proximal end 249 of the delivery catheter 203 (see FIG. 3F) can be connected or fixed to the housing body 220 of the proximal handle 201. The delivery catheter 203 can be attached to the proximal handle 201 by an adhesive or other mechanical connector. In embodiments in which the delivery catheter 203 has a lumen 225 (see FIG. 3D), the joint between the delivery catheter 203 and the proximal handle 201 can be hemostatic. The proximal and distal handles 201, 202 can be ergonomically-shaped such that the clinician can easily grip the handles 201, 202 during delivery of the pump 2. In an unlocked configuration, the distal handle 202 can slide proximally along the delivery catheter 203 from at least the distal end of the delivery catheter 203 to a location distal the proximal handle 201. The delivery system 200 may also include a transfer stop 208 as described below. The delivery system 200 may be configured such that the device (e.g., the pump 2) and the proximal handle 201 are put into a desired configuration by bringing various combinations of the proximal handle 201, the transfer stop 208, and the distal handle 202 together. The proximal handle 201, transfer stop 208, and distal handle 202 may have features (e.g., interlocking features) to facilitate arranging the components in these configurations.

The delivery catheter 203 can comprise an elongated member that is secured within the proximal handle 201. The delivery catheter 203 may be solid or may contain one or more central lumens 225 (see FIG. 3D). In the illustrated embodiments that include a retrieval lead, as explained herein, the catheter 203 can comprise one or more lumens 225. The delivery catheter 203 can be made from a material with sufficient pushability (to push the device down the delivery sheath) and flexibility (to track the bends in the delivery sheath). If the delivery catheter 203 comprises one or more central lumens 225, the lumen(s) 225 can be sized so the pump 2 or other medical device does not fit into the lumen(s) 225. Sizing the lumen(s) 225 of the delivery catheter 203 in such a manner can ensure that the delivery catheter 203 can push the pump 2 and prevent translation of the pump 2 in a proximal direction past the distal end of the delivery catheter 203. Delivery catheter cap 222 may be used to strengthen the end of delivery catheter 203 and maintain its round shape. In some embodiments, the delivery catheter 203 comprises a central lumen 225 that may contain the lead 20, 20A of the device (e.g., pump 2) that is to be placed in the anatomy. In some embodiments, the lead 20, 20A may be temporarily fixed (e.g., releasably fixed or connected) inside the proximal handle 201. In such embodiments, this arrangement allows the device (e.g., pump 2) to be both pushed and pulled by translation of the proximal handle 201. In some embodiments, the delivery catheter 203 may be made of a material that is transparent to visible light. The transparent delivery catheter 203 can allow visual inspection for bubbles during flushing and can allow visual inspection of the lead 20, 20A (e.g., to identify defects or to read markings on the lead 20, 20A like a serial number). The length of the delivery catheter 203 may be adjusted to match the parameters for different uses. For example, the length of the delivery catheter 203 may be in a range of 10″ to 40″, or in a range of 20″ to 35″ in various embodiments.

The delivery catheter 203 can extend within a cavity or lumen of the distal handle 202 (see, e.g., cavity 223 of FIG. 3B). The distal handle 202 can have a distal handle lock 214 having a locked configuration and an unlocked configuration. The clinician can move (e.g., rotate) the distal handle lock 214 in a first direction to place the distal handle 202 in the locked configuration, and can move (e.g., rotate) the distal handle lock 214 in an opposite second direction to place the distal handle 202 in the unlocked configuration. The delivery catheter 203 can be slidable relative to the distal handle 202 (or the distal handle 202 can be slidable relative to the delivery catheter 203) in the unlocked configuration. The delivery catheter 203 can be slidably locked relative to the distal handle 202 in the locked configuration. As shown in FIG. 2F, a distal handle unlock indicator 212A can indicate that the distal handle 202 is in the unlocked configuration. In the illustrated embodiment, the distal handle unlock indicator 212A can comprise a color (e.g., green) indicating that the distal handle 202 is unlocked and that the distal handle 202 and delivery catheter 203 can slide relative to one another.

The proximal handle 201 can have a proximal handle lumen 246 therethrough (see FIGS. 3G-31) that is in fluid communication with a lumen 225 of the delivery catheter 203. The proximal handle 201 can further include a fluid port 204 configured to deliver fluid to the lumen 246 of the proximal handle 201 and to the delivery catheter 203. The delivery system 200 can include a stopcock 209 connectable to the fluid port 204. The clinician can delivery fluid (e.g., saline) to the lumen of the proximal handle 201 and the lumen 225 of the delivery catheter 203.

The proximal handle 201 can also have an electrical port 205 configured to electrically connect the power lead 20, 20A to an external control system (such as a console). As explained herein, the power lead 20, 20A that is connected to the blood pump 2 can releasably connect to the proximal handle 201. Beneficially, in some treatment procedures (such as high-risk percutaneous coronary intervention (PCI) procedures), the blood pump 2 can be powered through the lead 20, 20A with the lead connected to the electrical port 205 of the proximal handle 201. In other procedures, as explained herein, the lead 20, 20A can be released from the proximal handle 201 and connected to the external system, for example, by way of an extension lead 206 and a power lead adaptor 210. For example, the proximal end 21 of the lead 20, 20A can be inserted into the power lead adaptor 210, and the power lead adaptor 210 can be connected to the extension lead 206. The power lead adaptor 210 can electrically connect the electrical contacts 58A-58C of the power lead 20, 20A to corresponding contacts of the extension lead 206. In some embodiments, a torque wrench 207 can be used to tighten set screws in the power lead adaptor 210 to improve the electrical connection. The extension lead 206 can electrically connect to the control system, which can control operation of the pump 2. In other approaches, the lead 20, 20A can be connected to the control system directly without the adapter 210 and/or without the extension lead 206. Patients with longer term pumping needs, e.g., more than 4 hours or more than 6 hours, may benefit from removal of the introducer set 300 and delivery system 200 so that only the device and its lead are left in the blood vessel. This increases flexibility and improves flow in the blood vessel. Patients who may be discharged with the blood pump 2 or who may be allowed to be ambulatory with the pump inside or outside the care facility where the pump is inserted, may benefit from direct connection of the pump to a controller worn on the patients body.

The delivery system 200 can also include a transfer stop 208 disposed about the delivery catheter 203 between the distal handle 202 and the proximal handle 201 such that the transfer stop 208 is distal of the proximal handle 201 and proximal of the distal handle 202. The transfer stop 208 can comprise a transfer stop lock 215 (see FIGS. 2N-2O) having a locked configuration and an unlocked configuration. The delivery catheter 203 can be slidable relative to the transfer stop 208 in the unlocked configuration and slidably locked relative to the transfer stop 208 in the locked configuration. As shown in FIG. 2F, a transfer stop unlock indicator 213A can indicate that the transfer stop 208 is in the unlocked configuration. In the illustrated embodiment, the transfer stop unlock indicator 213A can comprise a color (e.g., green) indicating that the transfer stop 208 is unlocked and that the transfer stop 208 and delivery catheter 203 can slide relative to one another.

The blood pump 2 and power lead 20, 20A can be disposed within the delivery system 200 prior to deployment. For example, at least a portion of the blood pump 2 can be disposed in the cavity 223 of the distal handle 202 (see FIG. 3B). In some embodiments, at least a distal portion of the blood pump 2 can be positioned within a distal hypotube 211 of the distal handle. The pump 2 can extend proximally into the cavity 223 of the distal handle 202 from a location proximal a distal end of the hypotube 211. In some embodiments, the distal end of the pump 2 can be recessed by a distance between 5 mm to 10 mm from the distal end of the hypotube 211. The power lead 20, 20A can extend proximally from the pump 2 through the lumen 225 of the delivery catheter 203 (see FIG. 3B) to the proximal handle 201. As explained above, the proximal end 21 of the lead 20, 20A can be connected (e.g., releasably connected) to the proximal handle 201. In various embodiments, the delivery catheter 203 can be transparent or translucent to visible light such that the clinician can view the power lead 20, 20A within the lumen 225 of the delivery catheter 203. The distal end of the delivery catheter 203 can abut a proximal end 248 (see FIG. 3B) of the pump 2. The delivery catheter 203 can accordingly prevent the pump 2 from moving proximally towards the proximal handle 201, and can also be used to push against the pump 2 to advance the pump 2 to the treatment location. For example, if the distal handle 202 slides proximally, the delivery catheter 203 can prevent the pump 2 from moving proximally.

Turning to FIG. 2G, the distal handle 202 can be placed in the locked configuration by moving (e.g., rotating) the distal handle lock 214. A distal handle lock indicator 212B can indicate that the distal handle 202 is in the locked configuration. In the illustrated embodiment, the distal handle lock indicator 212B can comprise a color (e.g., red) that is different from the color (e.g., green) of the distal handle unlock indicator 212A indicating that the distal handle 202 is locked and that the distal handle 202 is slidably locked relative to the delivery catheter 203.

FIG. 2H shows the delivery system 200 of FIG. 2F in a locked configuration. In FIG. 2H, both the distal handle 202 and the transfer stop 208 are in locked configurations. The transfer stop 208 can be placed in the locked configuration by moving (e.g., rotating) the transfer stop lock 215. A transfer stop lock indicator 213B can indicate that the transfer stop 202 is in the locked configuration. In the illustrated embodiment, the transfer stop lock indicator 213B can comprise a color (e.g., red) that is different from the color (e.g., green) of the transfer stop unlock indicator 213A indicating that the transfer stop 208 is locked and that the transfer stop 208 is slidably locked relative to the delivery catheter 203. Accordingly, in FIG. 2H, the distal handle 202 and the transfer stop 208 are slidably locked relative to the delivery catheter 203.

The transfer stop 208 can be configured to push the pump 2 to the desired treatment location inside the distal end portion of the delivery sheath 301, which can be indicated by a radiopaque band of the distal end of the sheath 301. As shown in FIG. 2H, before delivery, the transfer stop 208 can be spaced apart from the distal handle 202 by a length L_{C}. The length L_{C} can be selected based on the particular treatment procedure, e.g., based on how far into the anatomy (e.g., into the vasculature) that the pump 2 is to extend. In some embodiments, the length L_{C} can be selected so as to position the pump 2 such that an outlet of the pump 2 is at a location in the descending aorta at an elevation of the patient superiorly relative to the elevation of an LI vertebral body, e.g., such that the outlet (e.g., second fluid ports 25) of the pump 2 is disposed at an elevation of the patient that is between the elevation of the LI vertebral body and a T10 vertebral body. In various embodiments, the length L_{C} can be in a range of 25″ to 35″, or in a range of 27″ to 31″, e.g., about 29″ in one embodiment.

FIG. 2I illustrates a proximal end portion of the delivery sheath 301. The dilator 302 can be disconnected from the delivery sheath 301 by providing relative rotation between the dilator hub 303 and the delivery sheath hub 307. The dilator 302 can be withdrawn from the delivery sheath 301. The lumen 308 of the delivery sheath 301 can be flushed by way of the luer extension 305. Turning to FIGS. 2J and 2K, the distal handle 202 can be connected to the delivery sheath 301. The distal hypotube 311 can be inserted into the lumen 308 of the delivery sheath 301 across a hemostatic valve of the delivery sheath 301. The pump 2 (or at least a portion thereof) and a portion of the delivery catheter 301 can emerge across the hemostatic valve of the delivery sheath 301. In FIGS. 2J-2K, a distal connector 221 of the distal handle 202 can engage with the delivery sheath hub 307 by way of a quick-connect mechanism (e.g., by way of a bayonet-style connection in which the distal handle 201 and connector 221 are rotated to mate with the hub 307).

In FIGS. 2L and 2M, the distal handle 202 can be moved to the unlocked configuration, such that the distal handle unlock indicator 212A is shown. As noted above, in the unlocked configuration, the distal handle 202 and delivery catheter 302 can slide relative to one another. In FIGS. 2N-2P, the transfer stop 208 in the locked configuration can be advanced distally by the clinician towards the distal handle 202. Because the transfer stop 208 is slidably locked relative to the delivery catheter 302, the distal advancement of the transfer stop 208 causes the delivery catheter 302 to advance distally through the distal handle 202. During the distal advancement of the delivery catheter 302, a distal end 247 of the delivery catheter 302 (see FIGS. 3C-3D) can bear against and push a proximal end 248 of the pump 2 (see FIG. 3B) distally to advance the pump 2 and elongate lead 20, 20A (contained within the lumen of the delivery catheter) within the delivery sheath 301 to the target treatment location. As explained above, the length L_{C} can be selected such that, when the transfer stop 208 mates with the distal handle lock 214 of the distal handle 202, the pump 2 will have been advanced to a location near the distal end 311 of the delivery sheath 301, as shown in FIG. 2P. In FIG. 2P, the pump 2 may be situated within the delivery sheath 301 at a location that is at an elevation of the patient that is slightly superior to the desired operational treatment location, e.g., at an elevation of the descending aorta corresponding to an elevation between the LI vertebral body and a T10 vertebral body. The clinician can use standard imaging techniques to monitor the location of the pump 2 and/or distal end 311 of the delivery sheath 301 and to adjust the location of the pump 2 and/or sheath 301 before deployment of the pump within the descending aorta. The clinician can position the pump 2 without visualizing the blood vessels, but instead based on images of the vertebral bodies and the pump (which can include metal that can be readily imaged). For example, if the pump is positioned superiorly relative to the desired location, the clinician can lock the distal handle 202 and retract the distal handle 202 and transfer stop 208 to slightly retract the pump 2 and sheath 301 in an inferior direction to position the outlet(s) (e.g., the second fluid ports 25) of the pump 2 to be between the LI vertebral body and a T10 vertebral body.

Once the clinician has confirmed that the outlet(s) (e.g., ports 25) of the pump 2 are positioned at the desired location, in FIG. 2Q, the clinician can unlock the transfer stop 208 with the transfer stop lock 215 by rotating the transfer stop lock 215. If the distal handle 202 is locked, the clinician can also unlock the distal handle 202 with the distal handle lock 214 to ensure that both the distal handle 202 and the transfer stop 208 are in the unlocked configuration. With the distal handle 202 and the transfer stop 208 in the unlocked configuration, the distal handle 202 and transfer stop 208 can slide over the delivery catheter 301.

FIG. 2R shows the distal handle 202 and transfer stop 208 being moved proximally to mate with the proximal handle 201 of the delivery system 200. The clinician can anchor the proximal handle 201 (e.g., hold the proximal handle 201 still), which is affixed to the proximal end 249 of the delivery catheter 203 (see FIG. 3F). Anchoring the proximal handle 201 ensures that the pump 2 remains at the target operational location by causing the distal end 247 of the delivery catheter 203 to bear against the proximal end 248 of the pump 2. The clinician can retract the distal handle 202 and transfer stop 208 together proximally over the delivery catheter 203 towards the proximal handle 201. Since the distal handle 202 is connected to the delivery sheath 301 by way of the distal connector 221 and the delivery sheath hub 307, the proximal retraction of the distal handle 202 pulls the delivery sheath 301 proximally as well to unsheath the pump 2. The distal handle 202 can be retracted proximally until the transfer stop lock 215 mates with the proximal handle 201.

FIG. 2S is an image showing the blood pump 2 deployed in the descending aorta after the delivery sheath 301 is retracted proximally by an amount sufficient to deploy the pump 2. FIG. 2T is an image showing the blood pump 2 deployed in the descending aorta after the distal handle 202 and transfer stop 208 are fully retracted to the position shown in FIG. 2R to mate with the proximal handle 201 and retract the delivery sheath 301. Although not shown in FIG. 2T, the distal end 311 of the deliver sheath is still in the vasculature at this point in the procedure, disposed proximal the pump 2. FIG. 2T can also be the operational location of the pump 2 after the delivery system is removed. As shown in FIG. 2T, in the target location, the outlet(s) (e.g., second fluid ports 25) may be disposed between the LI and T12 vertebral bodies. Positioning the outlet(s) of the pump 2 at this location can position the outlet(s) above the renal arteries, e.g., between the renal artery and the left subclavian artery. Mapping the pump 2 relative to the vertebral bones, however, may be easier to visualize with fluoroscopic imaging techniques.

If the clinician is unsatisfied with the placement of the pump 2 in the anatomy, the clinician can re-sheath the pump by unlocking the distal handle 202 and the transfer stop 208. The clinician can hold the distal handle 202 stationary while retracting the proximal handle 201 proximally to re-sheath the pump 2 in the delivery sheath 301. The clinician can move the pump 2 (and/or the delivery sheath 301) until the pump 2 and/or distal end 311 of the deliver sheath 301 are in the correct location. The pump 2 can be re-deployed as shown in FIG. 2R.

In some arrangements, the pump 2 can be operated with the delivery system 200 still in place in the anatomy. As explained above, for some procedures (such as high risk PCI), the blood pump 2 can be powered through the lead 20, 20A with the lead connected to the electrical port 205 of the proximal handle 201. In other procedures, the delivery system 200 can be removed from the anatomy, and the lead 20, 20A can connect to the external control system as explained above.

FIGS. 2U-2X illustrate steps in a method for removing the delivery system 200 while leaving the pump 2 in place in the anatomy. In some cases, it may be difficult to remove the delivery system 200 from the vasculature due to friction, e.g., friction between the lead 20, 20A and the inner wall of the delivery catheter 203 and/or between the sheath 301 and the arteriotomy. As explained above, in some embodiments, the lead 20, 20A can comprise an elongate (e.g., extruded) insulator with lumen(s) formed therein and including the jacket 75 at an outside surface comprising a polyurethane insulating material, which can significantly reduce friction between the lead 20, 20A and the inner wall of the delivery catheter 203. Nevertheless, it still may be desirable to stabilize the pump 2 during removal of the delivery sheath 301 to compensate for the friction. FIG. 2U illustrates the insertion of a stiffening member 219 through the delivery system to the pump. The stiffening member 219 can comprise any suitable elongate member that can sustain a compressive force and/or exert a pushing force against a portion of the pump 2, e.g., against the proximal end 248 of motor (e.g., the proximal end of the motor housing 9) of the pump 2. The stiffening member 219 can comprise a guidewire in some embodiments. The stiffening member 219 can be advanced distally through the lumen 246 of the proximal handle 201 and the central lumen 55 of the lead 20, 20A to the proximal end 248 of the pump. The stiffening member 219 can bear against the pump 2 during removal of the delivery sheath 301.

Turning to FIG. 2V, as explained above, the proximal handle 201 can be releasably connected to the lead 20, 20A. The proximal handle 201 can comprise a lead release assembly 218 that can be configured to release the lead 20, 20A from the proximal handle 201. The lead release assembly 218 can comprise one or a plurality of actuators. In the illustrated embodiment, the lead release assembly 218 can comprise a first actuator 216 and a second actuator 217. The first actuator 216 can comprise a lead unlock button in the form of a sliding member. The clinician can slide the first actuator 216 distally to unlock the locking pin 245 from the locking recess 71 of the lead 20, 20A. As explained below, the first actuator 216 can comprise a catch 230 that prevents rotation of the second actuator 217 until the first actuator 216 is unlocked. As explained herein, in some embodiments, unlocking the locking pin 245 may be sufficient to enable the clinician to slide the proximal handle 201 proximally to separate the proximal handle 201 from the lead 20, 20A. However, in some embodiments, friction between the lead 20, 20A and internal components of the proximal handle 201 (e.g., the seal 243 and/or the terminals 244A-244D shown in FIGS. 3H and 3J-3K) may be sufficiently high such that the second actuator 217 can be used to release the lead 20, 20A from the proximal handle 201. In FIG. 2W, the clinician can rotate the second actuator 217 (e.g., a lead release knob) to separate the lead 20, 20A from the proximal handle 201 as explained herein.

Turning to FIG. 2X, with the distal handle 202 and transfer stop 208 locked to the delivery catheter 203 and the lead 20, 20A released from the proximal handle 201, the distal handle 202, the transfer stop 208, and the proximal handle 201 can be translated proximally while the stiffening member 219 is anchored or held in place. The delivery system 200 can be withdrawn until the delivery sheath 301 is outside the body. The clinician can connect the power lead 20, 20A to the external control system as explained herein, and the treatment procedure can commence with the pump 2 in place in the descending aorta and the power lead 20, 20A extending outside the body to the control system.

FIGS. 3A-3J show various features of the delivery system 200 described above in connection with FIGS. 2F-2X. Unless otherwise noted, the features of FIGS. 3A-3J may be the same as or generally similar to like-numbered components of FIGS. 2F-2X. For example, FIG. 3B is a schematic side sectional view of the distal handle 202. The distal handle 202 can have a handle body 224. As explained above, the pump 2 can be disposed in a handle cavity 223 of the distal handle 202. The pump 2 can be proximally retracted relative to the distal end of the distal hypotube 211 before delivery. As shown, the delivery catheter 203 can extend into the cavity 223 or lumen of the distal handle 202. A distal end 247 of the delivery catheter 203 can be disposed proximal of a proximal end 248 of a motor housing of the pump 2. When the delivery catheter 203 is advanced distally, the distal end 247 of the delivery catheter 203 can push against the proximal end 248 of the pump 2 to advance the pump 2 into the delivery sheath 301 to the target location.

As shown in FIGS. 3B and 3N, the distal handle lock 214 can engage a locking mechanism 259 to lock and unlock the inner catheter 203. An inner cap 255 can be disposed about the distal hypotube 211 and the delivery catheter 233. A seal member 256 can be disposed proximal the cap 255 within a proximal recess of the inner cap 255. A Tuohy seal 257 can be disposed within a cavity of the seal member 256 and can be disposed about the inner catheter 203. An o-ring 260 can be disposed about the inner delivery catheter 203 between the seal member 256 and the inner cap 255. Accordingly, the distal handle 202 can have various fluid isolation components that prevents fluid from leaking out. An inner knob portion 258 can be disposed within the lock 214 and connected thereto. The clinician can rotate the lock 214 to impart rotation to the inner knob portion 258, the seal member 256 and the Tuohy seal 257. In the locked configuration, the Tuohy seal 257 can be reduced in diameter to apply a compressive force and/or to exert a torque against the catheter 203 to inhibit sliding between the catheter 203 and the handle 202. In the unlocked configuration, the Tuohy seal 257 may apply a lesser or no compressive force and/or may not exert a positive torque against the catheter 203 such that the catheter 203 can slide relative to the handle 202. The lock 214 can be a Tuohy Borst adapter in some embodiments.

As explained above, the delivery catheter 203 can have a lumen 225 through which the lead 20, 20A may extend. Fluids can also be conveyed along the lumen 225 to flush the delivery catheter 203. As shown in FIGS. 3B-3D, a cap 222 can be disposed at the distal end 247 of the delivery catheter 203. The cap 222 can comprise a metallic cap having an opening at the distal end. Beneficially, the cap 222 can serve to strengthen the distal end 247 so as to maintain the shape of the catheter 203 during use. For example, in some cases, without the cap 222, the distal end 247 may press against the proximal end 248 of the pump 2 with sufficient force, such that the distal end 247 of the catheter 203 deforms or expands to partially fit over the proximal end of pump 2. The cap 222 can serve to stiffen the distal end 247 so that the catheter 203 does not deform. Further, as shown in FIG. 3E, and as explained above, in some embodiments, the delivery catheter 203 can comprise a material that is transparent to visible light. The clinician can visually inspect the power lead 20, 20A through the delivery catheter 203, for example, to confirm a serial number of the lead and/or to ensure the lead 20, 20A appears structurally sound.

FIG. 3F is a schematic perspective exploded view of the proximal handle 301. FIG. 3G is a schematic side sectional view of the proximal handle 301 of FIG. 3F. FIG. 3H is an enlarged schematic side sectional view of the proximal handle 301 of FIG. 3G. As explained herein, a proximal end 249 of the delivery catheter 203 can be connected to the proximal handle 201. As shown in FIG. 3F, the proximal end 240 can be adhered to a fluid manifold 226 of the proximal handle 201 with an adhesive. In other embodiments, the proximal end 240 of the catheter 203 can be attached or connected to the proximal handle 201 using any suitable sort of mechanical connector. The proximal handle 201 can include a distal cap 227 that mates with the fluid manifold 226 over the proximal end 249 of the delivery catheter 203. In use, the distal cap 227 may also mate with the transfer stop 208, e.g., during deployment of the pump 2.

In FIG. 3F, the housing body 220 can comprise multiple operational components disposed in or on a handle shell that includes a plurality of (e.g., two) shells 228A, 228B. The shells 228A, 228B can be connected by one or more fasteners 232. As shown in FIGS. 3F-3G, the fluid manifold 226 can provide fluid communication between the fluid port 204 and the lumen 225 of the delivery catheter 203. The fluid manifold 226 can be connected to a plunger 229 disposed proximal of the fluid manifold 226. As explained below, a seal 243 in the plunger 229 can prevent fluid from flowing proximally into the plunger 229.

The plunger 229 can serve to mechanically and electrically connect the housing body 220 of the proximal handle 201 to the lead 20, 20A in a releasable manner. For example, the plunger 229 can connect the electrical port 205 with terminals 244A-244D (see FIGS. 3H and 3J-3K) that connect to the connector 23 of the lead 20, 20A. The plunger 229 can also mechanically couple to the lead release assembly 218, e.g., to a lead locking device 237 that includes the first actuator 216 and an elongate catch 230, and to the second actuator 217. As shown in FIGS. 3F and 3G, the elongate catch 230 can extend proximally from the first actuator 216.

FIG. 3I is a schematic side view of a lead retention device comprising a locking pin assembly 238, according to various embodiments. The locking pin assembly 238 can be part of the lead locking device 237 that locks the lead 20, 20A to the proximal handle 201. As shown in FIG. 3I, the locking pin assembly 238 can include a locking pin 245 and a spring 250 mechanically coupled to the (e.g., disposed around) the locking pin 245. During assembly, a string or thread can be inserted through the lumen 246 of the proximal handle 201. The string or thread can be pulled through the transverse opening 68 and pulled proximally to seat the proximal end 21 of the lead 20, 20A in the position shown in FIG. 3I. The locking pin 245 can extend into the recess or groove 71 to lock the lead 20, 20A so as to prevent longitudinal translation of the lead 20, 20A. The spring 250 can be in compression in the locked configuration so as to bear against a locking pin head 251. To unlock the lead 20, 20A using the lead locking device 237, the clinician can slide the first actuator 216 (e.g., the lead unlock button) distally to urge a shoulder 252 of the elongate catch 230 distally. The shoulder 252 can comprise a thinned portion of the catch 230 recessed sufficiently such that the spring 250 in compression bears against the locking pin head 251 to remove the locking pin 245 from the recess 71 of the lead 20, 20A. When the first actuator 216 is in the unlocked configuration, it may be possible to separate the proximal handle 201 from the lead 20, 20A. However, in some instances, there may be friction between the lead 20, 20A and various components of the handle 201, as explained below. In such instances, the second actuator 217 can be engaged to release the lead 20A, 20 from the proximal handle 201.

Turning to FIGS. 3H and 3J-3K, the electrical connector or port 205 can comprise a plurality of wires 241 that electrically connect to an electrical board 240 (such as a printed circuit board, or PCB). The electrical board 240 can comprise various electronic components to assist in controlling the operation of the pump 2. For example, the board 240 can comprise one or more of simple interconnections through traces, frequency blocking elements, passive elements (such as capacitors, resistors, etc.), etc. The wires 241 can connect to corresponding terminals 244A-244D by way of traces 242. The terminals 244A-244D can comprise ring-shaped conductive terminals that are springy such that, when the lead 20, 20A is disposed in the lumen 246, the terminals 244A-244D can press against corresponding contacts 58A-58C of the lead 20, 20C. Although four terminals 244A-244D and three contacts 58A-58C are shown, it should be appreciated that in various embodiments, the number of terminals and contacts can match. For example, in various embodiments, there may be four terminals 244A-244D and four contacts 58A-58D (with 58D being unillustrated herein). Alternatively, there may be three terminals 244A-244C and three contacts 58A-58C. Any suitable number of contacts and terminals may be provided. In addition, a seal 243 can be disposed distal the terminals 244A-244D between the terminals 244A-244D and the fluid manifold 226. The seal 243 can extend around the lumen 246 in a manner that prevents fluids from flowing through the terminals 244A-244D.

The seal 243 and terminals 244A-244D can press snugly against the lead 20, 20A. In some cases, as explained above, when the first actuator 216 is unlocked and the locking pin 245 is released from the groove or recess 71, frictional forces between the scal 243 and the lead 20, 20A and between the terminals 244A-244D and the lead 20, 20A may make it difficult to separate or release the proximal handle 201 from the lead 20, 20A. Accordingly, the lead release assembly 218 can further include the second actuator 217 that assists in overcoming the frictional forces imparted on the lead 20, 20A.

As shown in FIGS. 3F-3H, the plunger 229 can comprise a shank 231 that extends proximally and includes external threads. The shank 231 can be disposed within a receiver 233 of the second actuator 217. A hollow shaft 234 (e.g., a steel hypotube) can extend through the receiver 233 and the shank and can bear against the lead 20, 20A. A proximal end cap 235 can be connected to the proximal end of the shaft 234. As shown in FIGS. 3F-3H, a tab 236 can be coupled to the receiver 233 and disposed around the threaded shank 231. The tab 236 can comprise an inwardly-projecting tooth 239 that engages with the threads of the threaded shank 231. The tab 236 can be affixed to the second actuator 217 by inserting the tab 236 into an opening 254 of the receiver 233, as shown in FIGS. 3L and 3M.

When the first actuator 216 is in the locked position, as shown in FIGS. 3H-31, the elongate catch 230 can fit within a notch 253 disposed on an outer surface of the receiver 233, as shown in FIGS. 3L and 3M. With the first actuator in the locked position, the catch 230 can prevent rotation of the second actuator 217 by bearing against a rim 255 that defines the notch 253. When the first actuator 216 is moved to the unlocked position to unlock the locking pin 245, the catch 230 can be moved distally out of the notch 253 to enable rotation of the second actuator 217 and receiver 233. Accordingly, the catch 230 and tab 236 can serve to prevent the second actuator 217 from rotating until the first actuator 216 is unlocked.

To release the lead 20, 20A from the proximal handle 201 in a manner that overcomes the frictional forces described above, the clinician can rotate the second actuator 217, which also rotates the receiver 233 and the tab 236. As the tab 236 rotates, the tooth 239 pulls the shank 231 (and the rest of the plunger 229) proximally within the receiver 233. In order to assist in overcoming the friction, the shaft 234 can bear against the power wire 20, 20A in compression. Once the shank 231 is retracted into the receiver 233, the connector 23 can be freed from the seal 243 and terminals 244A-244D, and the lead 20, 20A can be separated from the proximal handle 201 without applying a significant force (e.g., approximately zero force, except only the drag between the pump lead and the inner surface of the deployment catheter). This method of moving the plunger 229 proximally while maintaining the lead 20, 20A stationary is beneficial because it puts little or no tension on the lead 20, 20A. Additionally, moving the connector 23 distally may impart distal movement to the pump since the slack in the lead 20, 20A generated by moving the connector 23 distally may not fit inside the delivery catheter 203. The disclosed embodiment avoids this complication.

FIG. 3O is a schematic perspective exploded view of the transfer stop 208 according to various embodiments. The transfer stop 208 can include a housing 262 disposed about an inner knob 261 and a Tuohy seal 263. The inner knob 261 can fit within the transfer stop lock 215. Rotation of the transfer stop lock 215 can impart rotation to the inner knob 261 and the Tuohy seal 263, which can restrict an opening of an elastic material that seals against the inner catheter 203 to lock it in place. In the unlocked configuration, the Tuohy seal 263 may permit the catheter 203 to slide relative thereto.

FIG. 3P illustrates an example of the proximal handle 201 that includes one or more first vent holes 276 and one or more second vent holes 277 disposed distal the first vent hole(s) 276. In FIG. 3P, the connector 23 of the lead 20, 20A is shown connected to the terminals 244A-D, such that the seal 243 seals against proximally-flowing fluid. FIG. 3Q illustrates the proximal handle 201 of FIG. 3Q after the lead 20, 20A has been released from the plunger 229 and proximal handle 201. If the lead 20, 20A is connected to the proximal handle 201 at the time of manufacture, it may be connected in a way such that the connector 23 is part of a seal (e.g., seal 243) that maintains hemostasis as shown in FIG. 3P. This may create or provide an area 278 in the proximal handle 201 (e.g., in the plunger 229) on the other side of the hemostatic seal 243 that is inaccessible to a sterilizing gas (such as ethylene oxide, EO or EtO), unless an alternate path is provided for the sterilizing gas to reach this area 278. An alternative path may prevent or limit hemostasis once the connector 23 is released and is no longer in place. In such cases, the proximal handle 201 may contain elements that block these alternative sterilizing gas pathways at some time after sterilization is complete. In some cases, the action that releases the connector 23 may also close the alternative sterilizing gas pathways. In other cases, the action that closes the alternative sterilizing gas pathways may be a separate and distinct action. The proximal handle 201 can include the vent holes 276 and/or 277 to allow full EO sterilization; these vent holes 276 and/or 277 can be in any suitable location of the handle 201 but may be placed such that the vent holes do not compromise hemostasis.

In the embodiment of FIGS. 3P-3Q, for example, the first vent hole(s) 276 can be disposed at a first proximal portion of the proximal handle 201, and the second vent hole(s) 277 can be disposed distal the first vent hole(s) 276. In the illustrated embodiment, the vent holes 276, 277 can be provided through a sidewall of the hollow shaft 234 described above. The vent holes 276, 277 can extend through the sidewall in a direction transverse to the longitudinal axis to communicate with the lumen 246. In other embodiments, the vent holes 276, 277 can be provided at any other suitable location of the proximal handle 201. In some embodiments, a proximal port 275 at the proximal end of the proximal handle 201 may be opened, such that the sterilizing gas can pass through the port 275, along the lumen 246, and into the area 278 by way of the second vent holes 277. Sterilizing gas can also pass through the first vent holes 276. In some embodiments, the port 275 may be closed, in which case the sterilizing gas can pass through the first vent holes 276, the lumen 246, and the second vent holes 277 to the area 278.

During operation of the pump 2, blood may move within the space between the inner catheter 203 and the lead 20, 20A, unless hemostasis is maintained, e.g., by way of the seal 243. As explained above, the plunger 229 can be withdrawn proximally to release the lead 20, 20A after delivery. Withdrawal of the plunger 229 can disable the seal 243 which may allow blood to flow into the area 278 and into the second vent holes 277. However, in the embodiment illustrated in FIG. 3Q, the first vent holes 276 can be spaced apart from the second vent holes 277 by a distance selected such that, proximal withdrawal of the plunger 229 causes a covering portion 279 of the plunger 229 to cover and/or occlude the first vent holes 276. The covering portion 279 can accordingly maintain hemostasis after separation of the lead 20, 20A from the proximal handle 201.

### III. Example Retrieval Systems

When the treatment procedure is complete, or if the clinician decides to replace the pump 2, a retrieval system 400 (or retrieval system 400A) can be used to remove the pump 2 from the anatomy. Because the power lead 20, 20A extends proximally from the pump 2 outside the body, it can be challenging to access the pump 2 with the lead 20, 20A in place. Moreover, the lead 20, 20A is flexible, such that it can be challenging to advance a sheath or dilator directly over the lead 20, 20A, since friction between the sheath and the lead 20, 20A may cause the lead 20, 20A to bunch up and/or kink. Accordingly, various embodiments disclosed herein can beneficially provide an effective way to remove the pump 2 from the anatomy with the power lead 20, 20A in place. As explained above, it should be appreciated that, although the illustrated embodiment is directed to the retrieval of the intravascular pump 2, the retrieval systems 400, 400A can be used to retrieve other types of percutaneous medical devices 502.

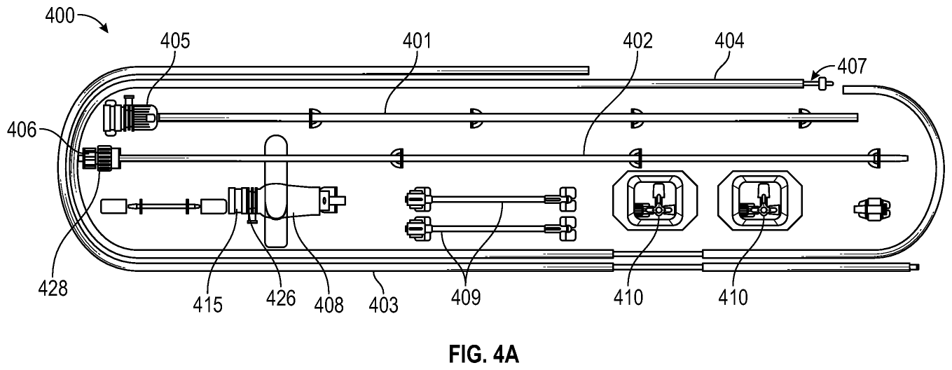

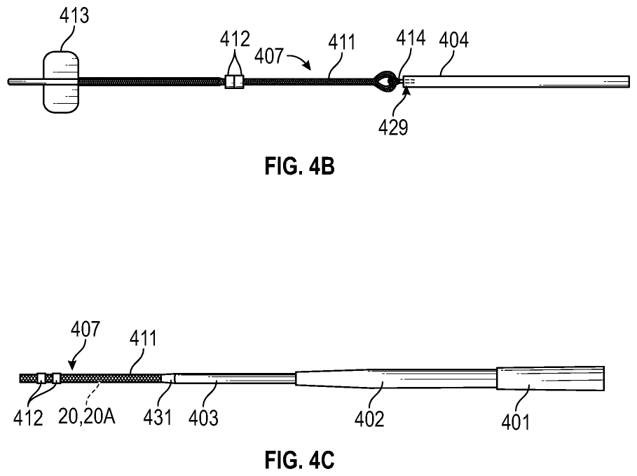

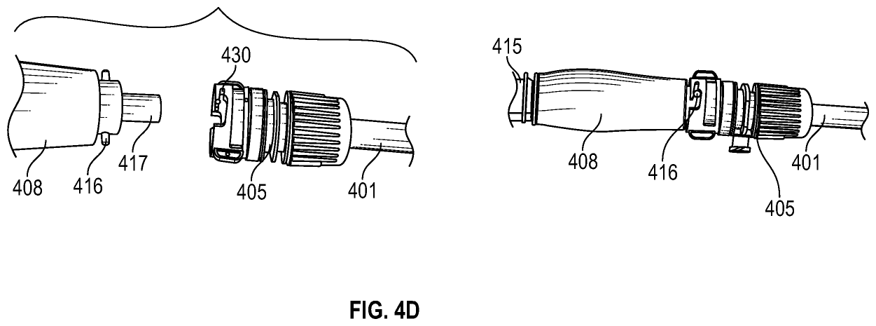

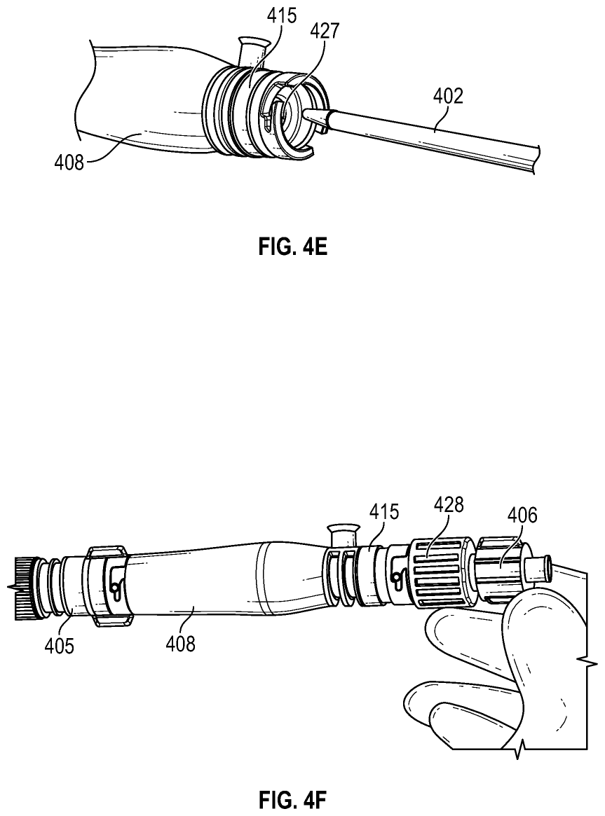

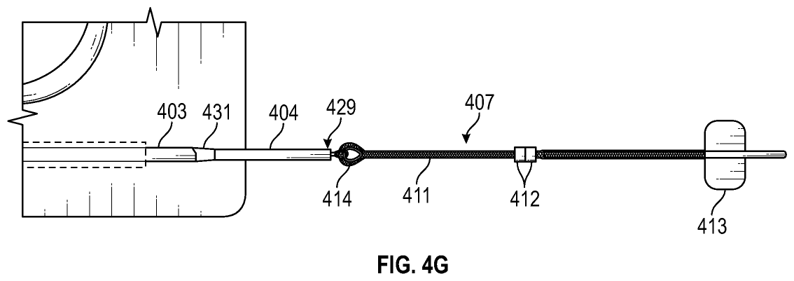

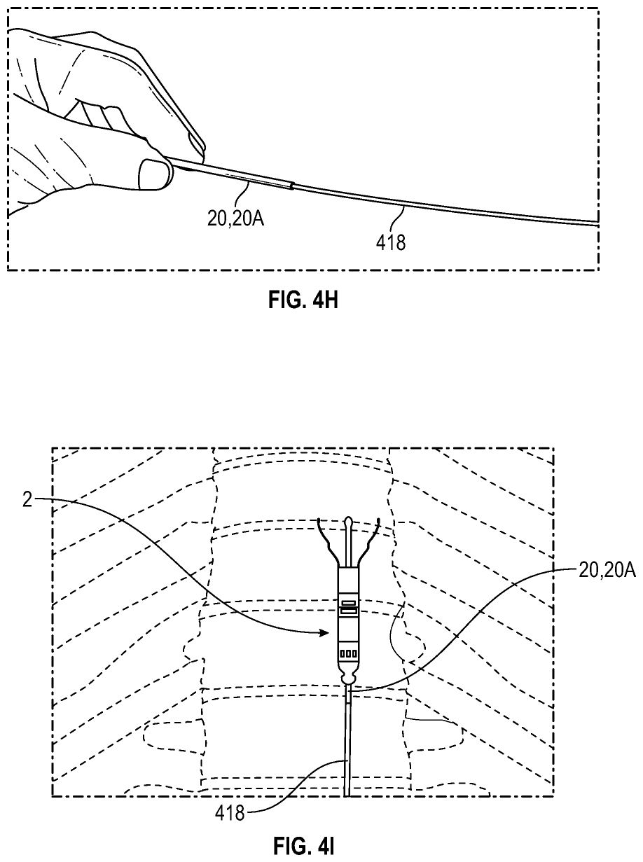

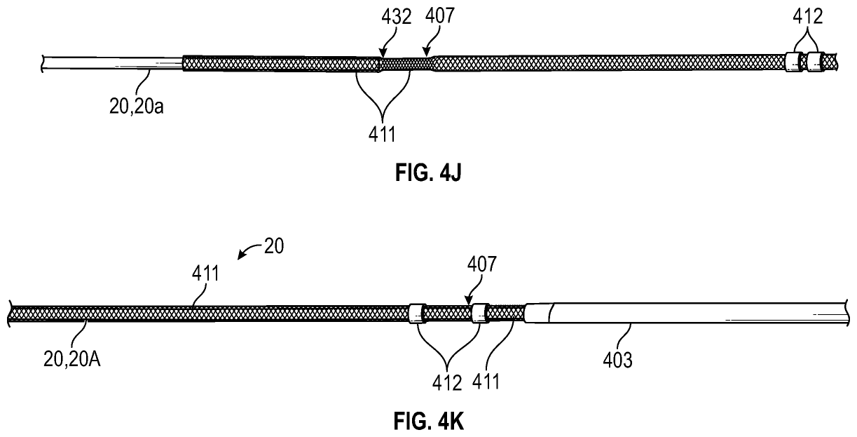

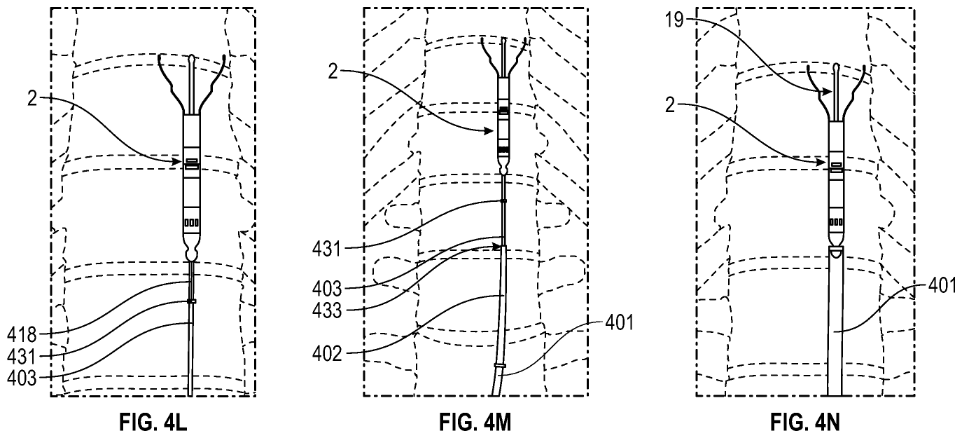

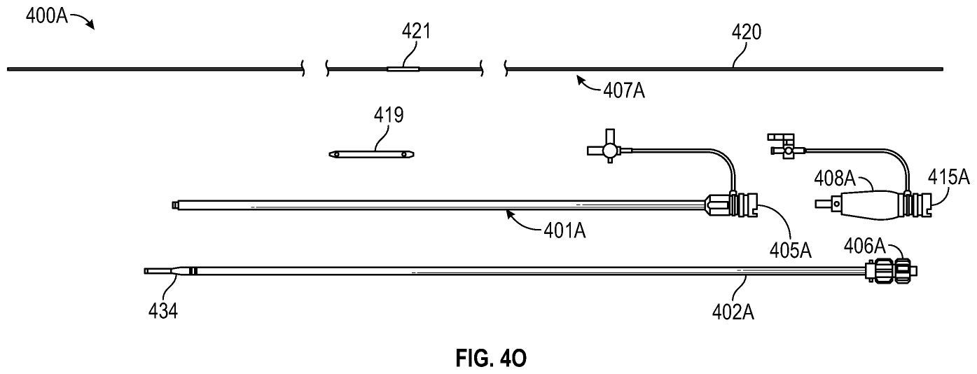

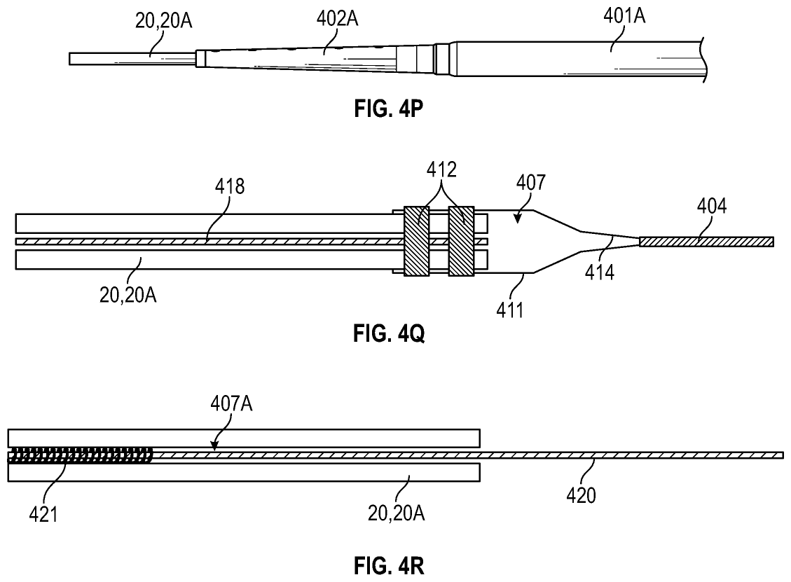

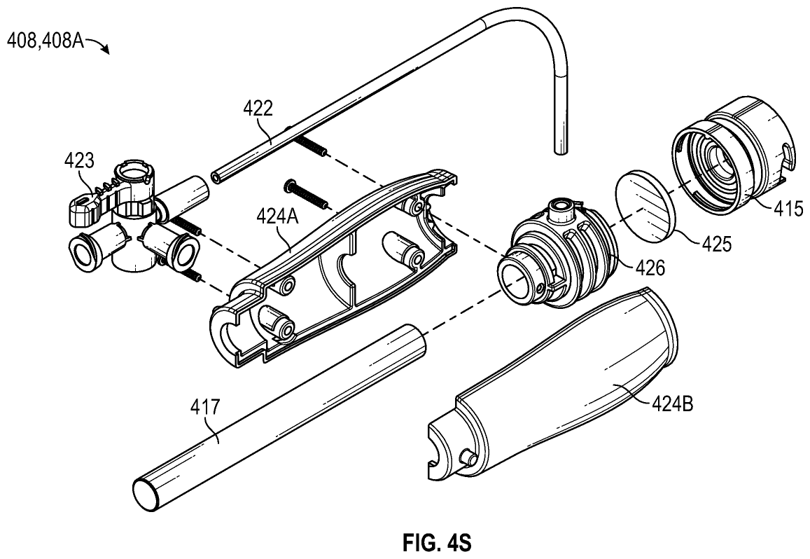

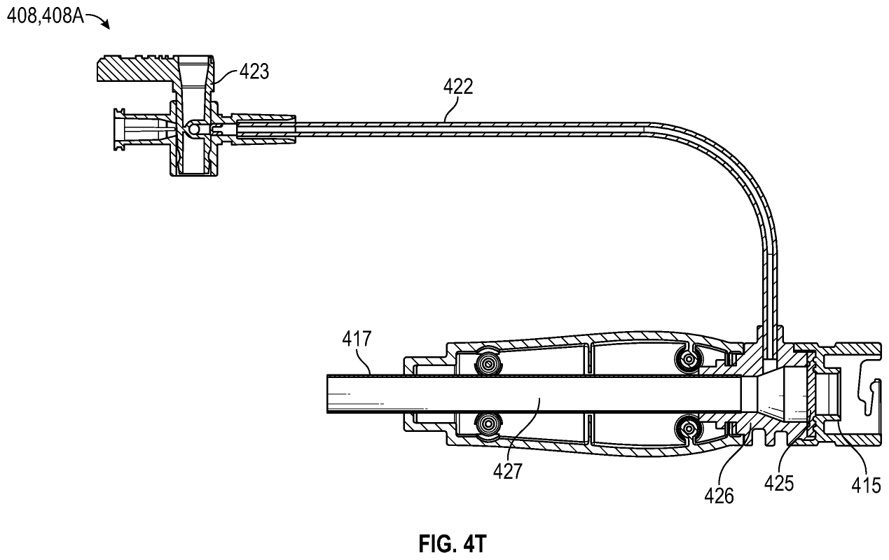

FIG. 4A illustrates a retrieval system 400, according to one embodiment. The retrieval system 400 can include a retrieval sheath 401 having a retrieval sheath hub 405 at a proximal end thereof, a retrieval dilator 402, a support catheter 403, a guide rod 404, and a retrieval handle 408. The retrieval dilator 402 can comprise a retrieval dilator hub 428 and a clamping member 406 (e.g., a Tuohy adaptor) at a proximal end of the dilator 402. The guide rod 404 can have a lead attachment device 407 connected to a distal end of the rod 404. The lead attachment device 407 can be configured to attach to a proximal portion of the lead 20, 20A to apply tension to the lead 20, 20A during removal of the pump 2. The retrieval handle 408 can have a retrieval handle proximal connector 415 at a proximal end of the handle 408 that is configured to connect to the dilator hub 428. The retrieval handle 408 can be configured to receive the pump 2 within an interior cavity (e.g., a retrieval handle lumen 427 as shown in FIG. 4T) of the handle 408 upon removal from the anatomy. The retrieval system 400 can also include one or multiple stopcocks 410 configured to provide fluid communication with the retrieval sheath hub 405 and/or a retrieval handle hub 426, by way of a corresponding one or plurality of luer extensions 409. The clinician can deliver fluid (e.g., saline) to flush the system components by way of the stopcocks 410.

FIG. 4B illustrates a lead attachment device 407, according to one embodiment. As noted above, it can be difficult to advance a sheath, dilator, or other hypotube over the elongate lead 20, 20A, because friction between the sheath and the lead 20, 20A may cause the power lead 20, 20A to bunch up and/or kink. Accordingly, the lead attachment device 407 can be provided to impart tension to the lead 20, 20A during retrieval. The lead attachment device 407 shown in FIG. 4B can include a retrieval cuff 411 connected to a distal portion 429 of the guide rod 404 by way of a cuff connector 414. One or a plurality of crimps 412 can be provided to secure the cuff 411 onto the lead 20, 20A. A retrieval stylet 413 can be provided within the cuff 411 in the package so as to maintain an opening within the cuff 411.

FIG. 4C illustrates an arrangement of components of the retrieval system 400 of FIG. 4A prior to retrieval. As shown in FIG. 4C, before retrieval, the lead attachment device 407 can connect to the proximal portion of the lead 20, 20A by way of the crimps 412 and to the guide rod 404 by way of the cuff connector 414. The guide rod 404 can extend through the support catheter 403 such that the distal portion 429 of the guide rod 404 extends distal the support catheter 403 before retrieval. The dilator 402 can be disposed over the support catheter 403, and the retrieval sheath 401 can be disposed over the retrieval dilator 402.

The clinician can connect a stopcock 410 and luer extension 409 to the retrieval handle hub 426 and can connect another stopcock 410 and luer extension 409 to the retrieval sheath hub 405. Turning to FIG. 4D, the clinician can insert a retrieval hypotube 417 of the retrieval handle 408 into a lumen of the retrieval sheath 401. The clinician can move (e.g., rotate) the retrieval handle 408 to connect the retrieval handle 408 to the retrieval sheath hub 405. For example, as shown in FIG. 4D, the rotation of the handle 408 can cause a retrieval handle distal connector 416 to rotate and connect to a corresponding slot of the retrieval sheath hub 405. Thus, in FIG. 4D, the retrieval handle 408 can be secured to the retrieval sheath 401 by way of the hub 405. The retrieval hypotube 417 can communicate with the lumen of the retrieval sheath 401. When the pump 2 is removed, the pump 2 can be withdrawn proximally into the hypotube 417 of the retrieval handle 408. The retrieval handle 408 and the retrieval sheath 401 can be flushed with heparinized saline.

Turning to FIGS. 4E-4F, the retrieval dilator 402 can be inserted into the retrieval handle lumen 427 of the retrieval handle 408 and advanced through the retrieval sheath 401. The dilator 402 can extend distal a distal end of the retrieval sheath 401. As shown in FIG. 4F, the retrieval dilator hub 428 can be locked with the retrieval handle proximal connector 415 by, e.g., rotating the hub 428. The clamping member 406 (which can comprise a Tuohy connector) can be loosened to enable relative sliding between the support catheter 403 and the retrieval dilator 402. Thus, after the step of FIG. 4F, the retrieval dilator 402 can extend through the retrieval handle 408 and the retrieval sheath 401. The dilator hub 428 and clamping member 406 can be disposed proximal the retrieval handle proximal connector 415.

The support catheter 403 can be flushed with saline, and the guide rod 404 can be inserted through the support catheter 403. As shown in FIG. 4G, the guide rod 404 can extend distal a marker band 431 at the distal end of the support catheter 403 prior to retrieval. The lead attachment device 407 can extend distal relative to the distal portion 429 of the guide rod 404. The clinician can power off the system 1 by, e.g., interacting with the external control system.

In FIG. 4H, a guidewire 418 can be inserted into the central lumen 55 of the power lead 20, 20A and advanced through the lumen 55 to the proximal end of the pump 2, as shown in FIG. 4I. Although a guidewire 418 is used in the illustrated embodiment, in other embodiments (such as those with leads that do not include lumens), a separate guidewire may not be used. The lumen 55 of the lead 20, 20A can comprise a blind hole that terminates at the back side of the pump 2, e.g., a wall of a housing of the pump 2 disposed proximal of the fluid ports 25. In some embodiments, the lumen 55 can have an opening at a proximal end of the lead 20, 20A and can be configured with no opening adjacent to or at the distal end of the lead 20, 20A or the pump 2. Thus, the guidewire 418 can be inserted through the lumen 55 and can abut a wall at the distal end of the lumen 55. The guidewire 418 can abut against the wall to apply a compressive force against the pump 2 to hold the pump 2 in place in vasculature while the support catheter 403 is advanced to the pump 2. Once the guidewire 418 is in place, the clinician can cut the power lead 20, 20A with a cutting tool, while leaving a sufficient length outside the anatomy to assist in retrieval. Cutting the power lead 20, 20A can remove the connector 23, such that the retrieval system 400 can remove the pump 2 without the connector 23 being present. Cutting provides that the remaining portion of the power lead 20, 20A lacks any steps, e.g., any radially outward steps, over a length extending to or from the proximal end to facilitate removal of the pump 2 with the retrieval system 400.

Turning to FIG. 4J, a cut proximal end 432 of the power lead 20, 20A can be inserted into the retrieval cuff 411. The retrieval cuff 411 can comprise a braided flexible material that can flex to receive the lead 20, 20A. As shown, the cuff 411 can extend along a length of the proximal portion of the lead 20, 20A. In FIG. 4K, the clinician can move the crimps 412 over the cuff 411 and the lead 20, 20A. As shown, the crimps 412 can be spaced apart along the lead 20, 20A by any suitable amount (e.g., by a distance in a range of about 5 mm to 10 mm for some procedures). The clinician can engage a crimping tool to deform the crimps 412 such that the crimps 412 press against the lead 20, 20A. In some embodiments, the crimps 412 can be deformed to press against both the lead 20, 20A and (indirectly, through the lead 20, 20A) the guidewire 418 within the lead 20, 20A. As explained herein, the lead attachment device 407 can apply tension to the lead 20, 20A during retrieval so as to prevent the lead 20, 20A from bunching up or kinking. For example, during retrieval, a clinician can apply tension to the guide rod 404, which is connected to the lead 20, 20A by way of the lead attachment device 407, while the clinician (or another clinician) advances the support catheter 403 over the lead 20, 20A. In FIG. 4K, the guide rod 404 is hidden because it is disposed within the support catheter 403, with the lead attachment device 407 extending distally out through the support catheter 403.

In FIG. 4L, the clinician can advance the support catheter 403 over the lead attachment device 407 (e.g., over the cuff 411 and crimps 412) and over the lead 20, 20A. As explained above, the guide rod 404 can be anchored by the clinician, while the support catheter 403 is advanced distally through the arteriotomy and into the femoral artery to the treatment location. With the lead 20, 20A anchored by the guide rod 404, the lead 20, 20A can serve as a rail over which the support catheter 403 can be guided to the pump 2. As shown in FIG. 4L, the support catheter 403 can be advanced to a location spaced proximally from the proximal end of the pump 2. For example, the distal marker band 431 can be positioned at a distance of 1 cm to 2 cm below the pump 2.

The clinician can flush the retrieval dilator 402 with saline. In FIG. 4M, the clinician can advance the retrieval dilator 402 and retrieval sheath 401 over the support catheter 403. To avoid kinking the support catheter 403 and/or power lead 20, 20A, the support catheter 403 and lead 20, 20A (by way of guide rod 404 and lead attachment device 407) can be anchored while the retrieval dilator 402 and retrieval sheath 401 are advanced together over the support catheter 403, e.g., by pushing the retrieval handle 408 distally. Beneficially, the support catheter 403 can serve as a rail over which the dilator 402 is advanced. Without the support catheter 403, the taper and tight fit between the dilator 402 and lead 20, 20A may impart high frictional forces which can cause the lead 20, 20A to bunch up. Accordingly, the support catheter 403 can serve as a guide along which the dilator 402 can be advanced. As shown in FIG. 4M, the retrieval dilator 402 and retrieval sheath 401 can be advanced until a distal end 433 of the dilator 402 is spaced apart proximally from the distal marker band 431 of the support catheter 403. In various embodiments, the distal end 433 can be spaced between 2 cm and 4 cm from the pump 2.

The clamping member 406 can have a clamped configuration in which the clamping member clamps against components passing therethrough (e.g., including at least the elongate lead 20, 20A) to prevent the components from sliding relative to the clamping member 406, and an unclamped configuration in which the components passing therethrough (such as at least the elongate lead 20, 20A) is slidable relative to the clamping member 406. The clamping member 406 of the retrieval dilator hub 428 can be tightened so as to press against and/or capture the power lead 20, 20A. In various embodiments, the clamping member can clamp against the support catheter 403, the lead 20, 20A, and the guidewire 418. Thus, the clamping member 406 (e.g., a Tuohy adaptor) can serve as a clamp that mechanically secures the retrieval dilator 402 to the power lead 20, 20A (and to the support catheter 403 and guidewire 418). Movement of the retrieval dilator 402 can accordingly impart movement to the support catheter 403, power lead 20, 20A, and guidewire 418.

The retrieval dilator 402 can be unlocked from the retrieval handle 408 by unlocking the retrieval dilator hub 428 from the retrieval handle proximal connector 415, which allows the retrieval dilator 402 (and, by virtue of the locked clamping member 406, the power lead 20, 20A, support catheter 403, and guidewire 418) to slide relative to the retrieval handle 408 and the retrieval sheath 401. In FIG. 4N, the clinician can hold the retrieval dilator 402 stationary (which also holds the power lead 20A, support catheter 403, and guidewire 418 stationary), and use the retrieval handle 408 to advance the retrieval sheath 401 over a proximal end of the pump 2. To remove the pump 2, the clinician can hold the retrieval handle 408 and retrieval sheath 401 stationary and can withdraw the retrieval dilator 402 proximally to pull the pump 2 into the retrieval sheath 401. As explained above, because the clamping member 406 locks against the power lead 20, 20A (and also the support catheter 403 and guidewire 418), proximal movement of the retrieval dilator 402 also imparts proximal movement to the power lead 20, 20A and pump 2. Withdrawal of the pump 2 into the retrieval sheath 401 can collapse the struts 19 and bring the pump 2 within the sheath 401, which can be verified with fluoroscopic imaging. The retrieval dilator 402 can be further retracted proximally until the pump 2 is retracted within the retrieval hypotube 417 within the retrieval handle 408.

Once the pump 2 is retracted into the retrieval handle 408, the clinician can unlock the retrieval handle 408 from the retrieval sheath hub 405. If indicated, the clinician can insert another guidewire for maintaining access and/or further procedures, and/or the retrieval sheath 401 can be removed from the patient. In some embodiments, however, the clinician can keep the retrieval sheath 401 in place, and can insert another pump 2 into the patient, for example, if the clinician decides that a replacement pump 2 should be provided for continued cardiovascular support. Accordingly, in some embodiments, the retrieval sheath 401 can remain in place so as to serve the function provided by the delivery sheath 301 described above.

FIG. 4O illustrates a retrieval system 400A, according to another embodiment. FIG. 4P illustrates an arrangement of components of the retrieval system 400A of FIG. 4O prior to retrieval. Unless otherwise noted, components in FIGS. 40 and 4P may be the same as or generally similar to like-numbered components of FIGS. 4A-4N, with the reference numbers appended by the letter “A.” For example, the retrieval system 400A can include a retrieval sheath 401A, a retrieval dilator 402A, and a retrieval handle 408A. A dilator stylet 419 can be provided within the dilator 402A to facilitate interaction with a seal of the sheath 401. Unlike the embodiment of FIGS. 4A-4N, however, the retrieval dilator 402A can comprise an integrated dilator in which the functionality of the support catheter 403 has been integrated with the functionality of the dilator 402.

The support catheter 403 provided in the embodiment of FIGS. 4A-4N is flexible enough to track and be advanced over the lead 20, 20A regardless of whether a central lumen 55 with or without a stiffening member (such as a guidewire or other elongate stiffening member) is present in the lead 20, 20A. This advancement may be facilitated by maintaining slight tension on the proximal end of the lead 20, 20A with the cuff 411 to prevent the lead 20, 20A from being dragged by the support catheter 403. Once the support catheter is in place, its stiffness and the lubricity of its outer surface allows easy advancement of the retrieval dilator 402 over the support catheter. By contrast, the integrated retrieval dilator 402A of FIGS. 40 and 4P can include a tapered distal portion 434 that conforms to the lead 20, 20A in a manner similar to that of the support catheter 403. The dilator 402A can accordingly have a stiffness sufficient to navigate the vasculature while also fitting with the lead 20, 20A in a manner that enables sliding between the lead 20, 20A and the dilator 402A. Thus, unlike the embodiment of FIGS. 4A-4N, the system 400A does not include a separate support catheter 403 to provide stiffness. Rather, the integrated dilator 402A can be advanced directly over the power lead 20, 20A, with the lead attachment device 407A providing sufficient stiffness and the lead 20, 20A material providing sufficiently low friction to prevent bunching up or kinking of the lead 20, 20A as the integrated dilator 402A is advanced.

Moreover, unlike the embodiment of FIGS. 4A-4N, in the embodiment of FIGS. 40-4P, the lead attachment device 407A can comprise a unified member that need not connect to a separate guide rod 404. As explained above, and as shown in FIG. 4Q, in the embodiment of FIGS. 4A-4N, the guidewire 418 can be inserted into the lead 20, 20A to provide compressive support against the pump 2. The lead attachment device 407 can include the cuff 411 disposed over the lead 20, 20A, and the crimps 412 clamped over the cuff 411 and the lead 20, 20A. A separate guide rod 404 can be provided to apply tension to the cuff 411 and the lead 20, 20A

By contrast, in the embodiment of FIGS. 40-4P, and as shown in FIG. 4R, the lead attachment device 407A can comprise an elongate stiffening element 420 and a locking element 421 disposed along the stiffening element 420. The elongate stiffening element 420 can also serve the function of the guidewire 418 of FIGS. 4A-4N, such that the separate guidewire 418 need not be provided. Rather, the clinician can insert the elongate stiffening member 420 into the lead 20, 20A and can advance the stiffening member 420 to the pump 2 to provide compressive support during retrieval. The integrated locking element 421 can be spaced along the stiffening element 420 at an intermediate location such that, when the stiffening element 420 bears against the pump, the integrated locking element 421 is disposed within the lead 20, 20A at a suitable location to lock with the lead 20, 20A. The locking element 421 can comprise any suitable locking stylet that provides a friction-fit connection with the interior wall of the lead 20, 20A. As with the guide rod 404 of FIGS. 4A-4N, during a retrieval procedure, the clinician can anchor the stiffening element 420 so as to apply tension to the lead 20, 20A during retrieval.

In other embodiments, the pump 2 can include an engagement feature 63 (see, e.g., FIG. 1J) disposed in a proximal portion of the pump 2 (e.g., at or near the retrieval feature 48). The engagement feature 63 can comprise internal threads, a bayonet connection feature or other mechanical couplings, that can engage with a suitable lead attachment device and that are in communication with the lumen 55. For example, the clinician can insert an elongate lead attachment device through the lumen 55 of the lead 20, 20A. The lead attachment device can comprise features that are complementary to the engagement feature 63 (e.g., complementary threads, bayonet features or other mechanical couplings). The clinician can advance the lead attachment device to the engagement feature 63 in the pump and can connect the lead attachment device to the engagement feature 63 (e.g., by a threaded connection, bayonet or other mechanical connection). As with the lead attachment devices 407, 407A, such a lead attachment device can beneficially be used to maintain tension in the lead 20, 20A during retrieval. Although the engagement feature 63 is shown in connection with FIG. 1J above, it should be appreciated that the engagement feature 63 can be provided in any of the pumps described herein.

Beneficially, the embodiment of FIGS. 40-4P and 4R can utilize fewer number of components as compared to the embodiment of FIGS. 4A-4N and can also enable a simplified procedure. For example, as compared with the embodiment of FIGS. 4A-4N, the system 400A of FIGS. 40-4P may not include a separate guidewire 418, guide rod 404, cuff 411, crimps 412, and support catheter 403. Rather, the integrated retrieval dilator 402A can accommodate the functionality of the support catheter 403. Similarly, the use of the locking element 421 integrated with the stiffening element 420 can perform the functions performed by the guidewire 418, the cuff 411, crimps 412, and guide rod 404 of FIGS. 4A-4N.

FIG. 4S is a schematic perspective exploded view of the retrieval handle 408, 408A, according to various embodiments. FIG. 4T is a schematic side sectional view of the retrieval handle 408, 408A of FIG. 4S. The retrieval handle 408, 408A can comprise a plurality of (e.g., two) handle shells 424A, 424B that can mate together with fasteners. A retrieval handle seal 425 can be provided within the handle 408, 408A between the proximal connector 415 and the retrieval handle hub 426. The retrieval hypotube 417 can extend into the retrieval handle hub 426 and distally past the handle shells 424A, 424B. As explained herein, the pump 2 can be retracted into the hypotube 417 during retrieval. As shown in FIGS. 4S and 4T, a flush port 422 can connect to the retrieval handle hub 426, and a stopcock 423 (e.g., a three-way stopcock) can be attached to the flush port 422.

### IV. Struts

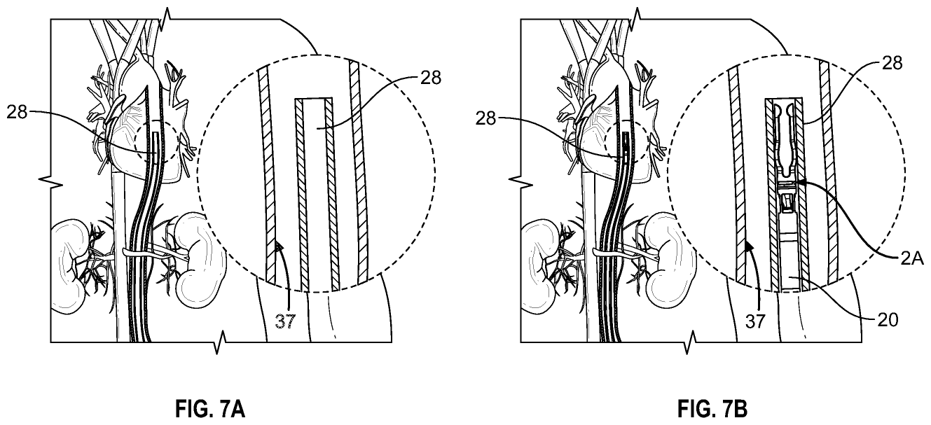

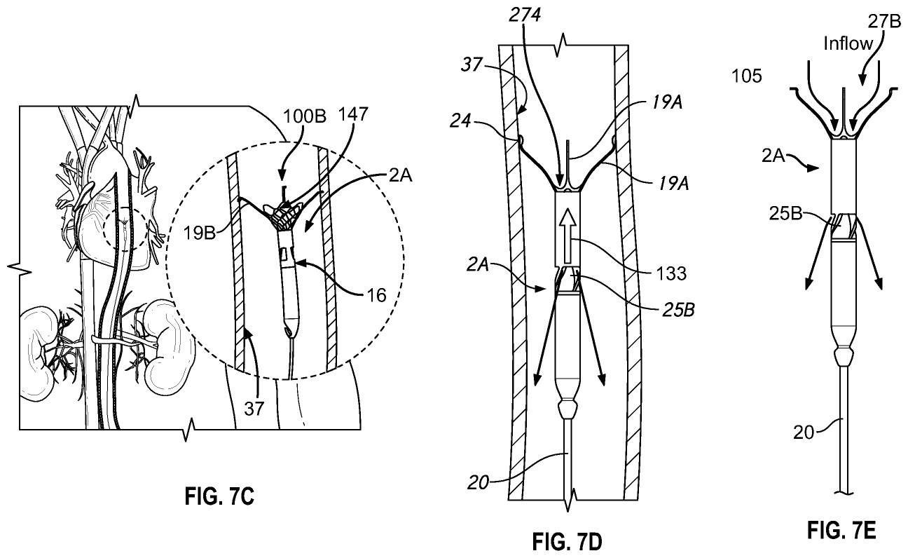

As explained herein, the support structure or localization system 100 can comprise a plurality of struts 19. Examples of struts may be found throughout International Application No. PCT/US2020/064489, filed Dec. 11, 2020, and in U.S. patent application Ser. No. 17/535,296, filed Nov. 24, 2021, the entire contents of each of which are incorporated by reference in their entirety and for all purposes. The struts 19 can have a first fixed end 38 at the base portion 36 that is coupled to or formed with the shroud 16, and a second free end 39 opposite the first end 38. The struts 19 can comprise projections extending from a housing (e.g., the pump housing 35) of a device, such as an intravascular device, extending radially and distally outwardly to make constant or intermittent contact with a vessel wall 37 (sec FIGS. 7A-7B) of a vasculature system of a patient. As explained above, in other embodiments, the struts 19 may extend proximally relative to the pump housing 35 and/or the motor housing 29. As shown in, e.g., FIGS. 1A-1C, the struts 19 can extend distal the first fluid port 27 and the impeller 6 along the longitudinal axis L. In embodiments in which the vasculature is accessed through the femoral artery, the struts 19 can extend distally and upstream of the first fluid port 27 and the impeller 6. In embodiments in which the vasculature is accessed through the subclavian artery, the struts 19 can extend downstream of the fluid port 27. The struts 19 can extend to and at least partially define a distal-most end of the blood flow assist system 1. In some embodiments, no portion of the blood flow assist system 1 is disposed distal the distal end of the struts 19. In some embodiments, the struts 19 may be made of a flexible shape set metal or alloy like nitinol. A support structure 100 including a plurality of struts 19 may be used to provide localization of an intravascular device such as the pump 2. Using a plurality of struts 19 allows each of the struts 19, by acting in opposition to each other, to transmit a radial force to the region of the strut 19 in contact with the vessel wall 37. A plurality of struts 19 may also be effective in positioning an intravascular device (such as the pump 2) or part of an intravascular device relative to the vessel wall 37. For example, a plurality of struts 19 surrounding the first fluid port 27 (e.g., an inlet port in some embodiments) of the intravascular pump 2 effectively positions the inlet port 27 of the pump 2 at approximately the center of the blood vessel 37. Struts 19 for localizing and positioning intravascular devices may have a collapsed configuration for moving through the sheath 28 (see FIG. 1H) for deployment or retrieval and an expanded configuration for providing localization and positioning.

FIG. 5A is an image showing a front perspective view of the localization system 100A, according to one embodiment. FIG. 5B is a schematic side view of the localization system 100A of FIG. 5A. FIG. 5C is a schematic plan view of a laser cut pattern for the localization system 100A. FIG. 5D is a schematic side plan view of a strut 19A having a dome- or spherical-shaped contact pad 24A. Unless otherwise noted, the components of FIGS. 5A-5D may be the same as or generally similar to like-numbered components of FIGS. 1A-1H, with some reference numbers appended by the letter “A.” As shown in, for example, FIGS. 5A-5D, each strut 19A can comprise an elongate slender body that extends between the first end 38 and the second end 39. Each strut 19A can comprise a material (e.g., a shape memory alloy) that is configured to store strain energy when a transverse compressive load is applied, e.g., compressively along the radial axis R. The stored strain energy can be employed to maintain localization and/or positioning relative to the vessel wall 37, as explained herein. For example, the stored strain energy can be result in radially outward forces being applied against the vessel wall 37. The radially outward forces can at least in part serve to localize, stabilize, and/or position the pump 2 relative to the vessel wall 37.

In some embodiments, a portion of the strut 19A that makes contact with the vessel wall 37 may have a desired shape that aids localization and/or positioning. In some embodiments, a portion of a strut 19A, such as its second end 39, may comprise a contact element 104 configured to be shaped as a generally flat contact pad 24A. In the illustrated embodiment, the contact pad 24A is shown as being generally circular or domed. Other shaped ends may be suitable, such as an oval end or the like. In some embodiments, shapes for the contact pad 24 that avoid sharp corners and/or edges may be preferred. When deployed, the contact pad 24A can be pressed against the wall 37 of the vessel with a radial force transmitted by the strut 19A. As the pad 24A presses against the vessel wall 37, the vessel wall 37 may “pillow” up around the edges of the pad 24A or the pad may form a depression in which it sits. The elongate struts can be configured to apply a load to the vessel wall 37 (e.g., an aortic wall) when deployed to locally radially expand vessel wall tissue against which the contact pad 24A is apposed. For example, the contact pad 24 can be resiliently deflectable toward and away from the longitudinal axis L of the pump housing 35. The contact pad 24 can have a free state being spaced away from the longitudinal axis L of the pump housing 35 by a distance greater than a half-width of a blood vessel 37 into which the pump housing 35 is to be deployed.

The contact pad 24 can apply sufficient force to a wall of the blood vessel 37 to depress or pillow a portion of the contact pad 24 into the wall. The contact pad 24 can be configured to engage without hooking the wall of the blood vessel 37 when applied. In some arrangements, the struts 19A can flex with vessel wall movement (e.g., with vessel wall expansion and contraction) such that the struts 19A can maintain contact with the vessel 37 even when the vessel 37 expands or contracts. This pillowing may enhance the ability of the strut 19A and pad 24A to localize the intravascular device (e.g., pump 2) by resisting sliding motion of the pad 24A. The amount that the pad 24A presses into the vessel wall 37 (and therefore the amount of pillowing) may be controlled by adjusting the radial force the strut 19A transmits to the contact pad 24A. The pad 24A may have holes or irregular edges to enhance the pillowing effect.

As shown in FIGS. 5E-5G, struts 19A′ may include contact pads 24B having “slide runner” edges 66 that flare or bevel away from the vessel wall 37 so that sharp edges are not pressed into the vessel wall 37. As shown in FIGS. 5E-5G, the contact pads 24B can include a contact surface 67 that engages and depresses into the vessel wall 37, such that a surrounding portion of the wall 37 extends radially inward relative to at least a portion (e.g., the contact surface 67) of the contact pad 24B that engages the wall 37. The profile of the pad 24B in FIGS. 5E-5G including the edge 66, the contact surface 67, and the elongate member of the strut 19A can define a convex profile or shape. In the illustrated arrangement, the contact surface 67 can comprise a generally planar or flat shape, and the edge 66 can extend at an obtuse angle relative to the contact surface 67. In some embodiments, the contact surface 67 can comprise a curved surface, such as a convex spherical or domed surface. Such designs reduce or minimize the potential for traumatic injury to the vessel wall 37, are non-endothelializing, and may aid removal without damaging the vessel. With sufficient radial force and pillowing, such designs may provide stable localization of the strut contact pad 24A.

As shown in FIGS. 5A-5B, the struts 19A can comprise knees 102 that can serve to keep the strut 19A away from the inner wall of the sheath 28 when the plurality of struts 19A is collapsed within the sheath 28, as shown above in FIG. 1H. The sheath 28 can comprise an inflection in which the curvature of the radially-outward facing surface of the strut 19A changes. As shown in FIG. 5B, for example, the struts 19A can comprise a plurality of segments 103a-103d that are integrally formed and connected with one another. A first segment 103a can extend from the base portion 36A distally and radially outwardly by an angle A relative to the longitudinal axis L. A second segment 103b can extend distally and radially inwardly from the distal end of the first segment 103a by an angle B relative to the longitudinal axis L. A third segment 103c can extend distally and radially outwardly from the distal end of the second segment 103b by an angle C relative to the longitudinal axis L. A fourth segment 103d can extend distally and radially inwardly from the distal end of the third segment 103c by an angle D relative to the longitudinal axis L.

Thus, as shown in FIG. 5B, the struts 19A can have multiple changes in curvature and/or angles along the lengths of the struts 19A. In various embodiments, the angle A can be in a range of 30° to 70°, in a range of 40° to 60°, or in a range of 45° to 55° relative to the longitudinal axis L. The angle B can be in a range of 10° to 30°, in a range of 15° to 25°, or in a range of 18° to 24° relative to the longitudinal axis L. The angle C can be in a range of 20° to 60°, in a range of 30° to 50°, or in a range of 35° to 45° relative to the longitudinal axis L. The angle D can be in a range of 20° to 45°, or in a range of 25° to 35° relative to the longitudinal axis L. The base portion 36A can have a first height H1 in a range of 0.1″ to 0.3″. In the expanded configuration, the radial separation along the radial axis R between the ends of the struts 19A can have a second height H2 in a range of 1″ to 2″, or in a range of 1.2″ to 1.6″.

Beneficially, the use of multiple angles and curvatures for the struts 19A can enable the struts 19A to provide sufficient localization and support for the pump 2. Additionally or alternatively, the use of multiple angles and/or curvatures for the struts can adequately space parts of the struts, for example the free ends of the struts 19A, from the inner wall of the sheath 28. The spacing of the pads 24A from the inside wall of the sheath 28 can reduce friction and/or damage to the struts 19A and/or sheath 28 when the pump 2 is moved within and/or into and out of the sheath 28. Further, as explained above, the flat contact pads 24A can beneficially provide an atraumatic interface between the struts 19A and the vessel wall 37 that provides sufficient localization and/or positioning. The struts 19A can be manufactured by laser cutting a shape memory alloy as shown in, e.g., the laser cut pattern in a sheet of material of FIG. 5C. The shape memory alloy (e.g., nitinol) can be cut with a laser or other device and shaped to form the struts 19A. The patterned material can be folded and/or rolled into a closed generally cylindrical profile. In other embodiments, the pattern can be cut from an already-formed tube.

As shown in FIG. 5C, one or a plurality (e.g., three) windows 61 can be formed in the base portion 36. The windows 61 can be configured to mate with corresponding flanges 62 of the shroud 16 and/or of a bearing member intervening between the shroud 16 and the support structure. As shown in FIG. 5A, the flanges 62 can be inserted in the windows 61 to secure the base portion 36 to the shroud 16. In some embodiments, the localization system 100A may be made of a different material from the shroud 16 (or other intervening bearing or structure). For example, in some embodiments, the localization system 100A can comprise a shape memory alloy (such as nitinol), and the shroud 16 (or other intervening structure) can comprise a different metal (such as titanium). Since it can be challenging to weld such different materials, the use of the flanges 62 and windows 61 can beneficially enable a secure connection between the localization system 100A and the shroud 16 (or other intervening structure) without using a weld. In addition, the base portion 36 can comprise a slit 64 as shown in FIG. 5C. The slit 64 can be formed to in the base portion 36 and can extend longitudinally. The slit 64 can be provided to deform so as to accommodate the tight fit when the flange 62 is inserted into the windows 61. Once the flanges 62 are inserted into the windows, the slit 64 can be welded closed.

In some embodiments, such as that shown in FIG. 5D, the contact pad 24A or distal portion of the strut 19A may include a spherical or domed-shaped profile 42 that serves as the contact surface 67. As a nonlimiting example, the spherical profile 42 may be formed as a ball of plastic or other material formed on the portion of the strut 19A to contact the vessel wall 37. As shown in FIGS. 5B-5D, for example, the spherical profile 42 can be disposed on a radially-outer surface 43 of the strut 19A that is configured to face and engage with the vessel wall 37. A radially-inner wall 44 can be disposed radially opposite the radially-outer surface 43. In FIG. 5C, the struts 19A can be circumferentially spaced apart such that there is a respective gap 45 between adjacent side surfaces of adjacent struts 19A of the plurality of struts 19A. A spherical contact feature 24A can be beneficially atraumatic, and may provide good pillowing and resistance to translation. As shown the contact pad 24A can comprise a generally circular (or elliptical) pad in a profile view that has a diameter greater than a width of an immediately adjacent expanse of the corresponding elongate strut 19A. The contact pad 24A can comprise an elongate member and an enlarged blood vessel wall contact surface (e.g., surface 67 in FIGS. 5D-5G) disposed at the end of the elongate member. In various embodiments, the contact pad 24A can comprise a convex cross-sectional profile along the radially-outer surface 43 of the strut 19A that faces the vessel wall 37. For example, the contact pads 24A can comprise a convex profile in a cross-sectional plane disposed transverse to the longitudinal axis L of the pump housing 35. In some embodiments, the contact pads 24A can comprise smooth surfaces free of sharp edges or hooks. In some embodiments, each of the contact pads 24A can comprise one or more scalloped edges to allow tissue of the vessel wall 37 to be received therein.

In some embodiments, the localization system 100A may have the goal of resisting, but not eliminating, the translation or rotation of a device (such as the pump 2) relative to the vessel wall 37. As a nonlimiting example, some strut 19A and/or contact pad 24A designs may allow some small degree of rotation of the device within the vessel, even when deployed. However, such designs may also leverage other features discussed herein to further increase resistance to rotation during operation of the device, such as increase resistance resulting from propulsion.

FIGS. 5H-5J illustrate additional examples of struts 19E-19G that can provide for improved pillowing into a blood vessel wall. It should be appreciated that although one strut is shown in each of FIGS. 5H-5J, multiple struts can be provided for each system. FIG. 5H illustrates a contact element 104 at a distal end of a strut 19E, according to another embodiment. The contact element 104 can comprise a contoured profile to facilitate contact with the blood vessel wall. In FIG. 5H, the contact element 104 can have a blunt distal edge 135 in some embodiments. In FIG. 5H, the contact element 104 can have a generally planar surface angled relative to the thin filament of the strut 19E in some embodiments. In other embodiments, the contact element 104 can comprise a convex surface as explained above.

FIG. 5I illustrates a strut 19F having a contoured profile having a contact surface 122A in which a plurality of fingers 120 are spaced apart by an opening 121 (e.g., a plurality of openings) in the contact surface 122A. The plurality of spaced apart fingers 120 can provide an increased surface area, which can improve the pillowing effect into the blood vessel wall. Similarly, in FIG. 5J, the strut 19G can include a contoured profile with a contact surface 122B having a plurality of openings 123 in the contact surface 122B. Alternatively, the contact surface 122B can have a plurality of protrusions extending from the contact surface 122B. The openings 123 (or protrusions) can provide an increased surface arca for improved pillowing.

FIG. 5K is a schematic perspective view of a shroud 16 having a plurality of twisted struts 19H extending therefrom. FIG. 5L is an axial end view of the struts 19H of FIG. 5K. As shown in FIGS. 5K-5L, the strut 19H can have a contact element 104 at a distal portion thereof and an elongate member 124 extending between the contact element 104 and the pump housing or shroud 16 along an axis of the strut. The elongate member 124 can have a cross-section taken perpendicular to the axis of the strut 19H that varies rotationally along a length of the strut 19H. For example, as shown in FIGS. 5K-5K, the elongate member can be twisted about the axis of the strut. The use of a twist in the strut 19H can advantageously improve the deformation resistance of the strut 19H as well as present a more streamlined profile with less resistance to blood flow.

Alternatively, some embodiments of the contact pad may by designed to increase resistance to translation and/or rotation relative to the vessel wall 37. FIG. 6A is an image of a front perspective of a localization system 100B according to another embodiment. FIG. 6B is an image of a side view of the localization system 100B of FIG. 6A. FIG. 6C is a schematic side view of the localization system 100B of FIGS. 6A-6B. FIG. 6D is a schematic enlarged view of the second end 39 of the strut 19B of FIGS. 6A-6C. FIGS. 6E and 6F are schematic plan views of the localization system 100B in a laser cut pattern prior to assembly. Unless otherwise noted, the components of FIGS. 6A-6F may be the same as or generally similar to like-numbered components of FIGS. 1A-11 and 5A-5C, with some reference numbers appended by the letter “B.” In some embodiments, the contact element 104 (e.g., the portion of the strut 19B in contact with the wall 37 of the vessel) may comprise a hook 105 designed to penetrate the vessel wall 37 to provide a stable anchor point that has a high level or resistance to translation and/or rotation. Designs with edges or hooks 105 in constant contact with the vessel wall are typically intended to provide stable localization and/or positioning so there is little or no motion of the hook 105 or edge relative to the initial contact region of the vessel wall 37 when deployed.

As shown in FIG. 6C, the struts 19B can comprise a plurality of segments 106a-106d that are integrally formed and connected with one another. A first segment 106a can extend from the base portion 36B distally and radially outwardly by an angle E relative to the longitudinal axis L. A second segment 106b can extend distally and radially inwardly from the distal end of the first segment 106a so as to at least partially define an inflection point and/or knee 102 as explained above. A third segment 106c can extend distally and radially outwardly from the distal end of the second segment 106b by an angle F relative to the longitudinal axis L. A fourth segment 106d can extend back proximally from the distal end of the third segment 106c by an angle G relative to the third segment 106c. The third and fourth segments 106c, 106d can serve as the hook 105 and can secure the pump 2 to the vessel wall 37. As shown in FIG. 6G, which is a plan view of the fourth segment 106d, the fourth segment 106d of the strut 19B can include a split 106e having tines that can secure to a vessel wall, in some embodiments. As shown, in some embodiments, a tine width t_{w} can be in a range of, e.g., 0.01″ to 0.1″, or in a range of 0.01″ to 0.05″.

As shown in FIG. 6C, the struts 19B can have multiple changes in curvature and/or angles along the lengths of the struts 19B. In various embodiments, the angle E can be in a range of 30° to 70°, in a range of 40° to 60°, or in a range of 45° to 55° relative to the longitudinal axis L. The angle F can be in a range of 20° to 60°, in a range of 30° to 50°, or in a range of 35° to 45° relative to the longitudinal axis L. The angle G can be in a range of 40° to 80°, in a range of 50° to 70°, or in a range of 55° to 65° relative to the segment 106c, angled proximally as shown. The base portion 36B can have a first height H1 in a range of 0.1″ to 0.3″. In the expanded configuration, the radial separation along the radial axis R between the ends of the struts 19B can have a second height H2 in a range of 1″ to 2″, or in a range of 1″ to 1.4″. Further, as shown in FIG. 6C, the knee 102 can have a bump height h_{b} that indicates the amount of the bulge or bump defined by the knee 102. The bump height h_{b} can be measured between an outwardly-facing crest of the knee 102 and a projection of the third segment 106c. In various embodiments, the bump height h_{b} can be in a range of 0.03″ to 0.09″, or in a range of 0.05″ to 0.07″ (e.g., about 0.054″ in one embodiment). In addition, the fourth segment 106d can serve as a tine of the hook 105 and can have a tine length lₜ extending proximally from the third segment 106c. The tine length l, can be in a range of 0.03″ to 0.09″, or in a range of 0.05″ to 0.07″ (e.g., about 0.058″ in one embodiment).

FIGS. 6E-6F show laser patterns for the system 100B of FIGS. 6A-6D. As shown in FIGS. 6E-6F, in some embodiments, the struts 19B can be tapered across their width from proximal to distal along their length, i.e., from right to left in FIGS. 6E-6F. Laser cuts can be made non-normal to the longitudinal axis, which can create a helical or spiral pattern in various arrangements.

FIG. 6H is a schematic side view of a plurality of struts 19I in expanded and collapsed configurations relative to a sheath 28, according to another embodiment. As explained herein, the struts 19I can comprise a shape memory alloy (such as nitinol) with superelasticity so as to enable the struts 19I to have expanded and collapsed states. FIG. 6I is a schematic side view of an example strut 19I engaging a vessel wall 37. FIG. 6J is a front view of the struts 19I disposed within the sheath 28. The strut 19I can have a plurality of segments 125a, 125b integrally formed and connected with one another. The plurality of segments comprising a first segment 125a extending distally and radially outwardly relative to the longitudinal axis and a second segment 125b extending proximally and radially outwardly from a distal end of the first segment 125a, the second segment 125b configured to at least intermittently engage the blood vessel wall 37. In addition, some struts 19I may have a second segment 125c that extends radially and distally outwardly relative to the first segment 125a. In various embodiments, the system may include struts with proximally-extending second segments 125b and with distally-extending second segments 125c. In some embodiments, the system may only include proximally-extending second segments 125b. In some embodiments, the system may only include distally-extending second segments 125c.

As shown in FIG. 6I, the first segment 125a can be angled relative to the shroud 16 by an obtuse angle a (e.g., in a range of 90° to 180°, or in a range of 95° to 165°). The first and second segments 125a, 125b can be angled relative to one another by an acute angle b (e.g., in a range of 10° to 80°). The second segment 125b can contact the vessel wall 37 at a location c. Further, as shown in FIG. 6J, the first and second segments can meet at a joint 125d. In a collapsed configuration, such as that shown in FIG. 6J, the joint 125d of each strut 19I can be disposed radially inward within the sheath 28.

FIG. 6K illustrates a strut having connected to a shroud 16, according to another embodiment. The strut can comprise a curved expanded profile 126. In the illustrated embodiment, the curved expanded profile 126 can curve back proximally relative to the shroud 16. The proximally-curving expanded profile 126 can have an end 126a that curves radially inward towards the shroud 16. The radially inward extending end 126a can serve as an atraumatic end of the curved expanded profile 126 so as to avoid damaging the vessel wall 37.

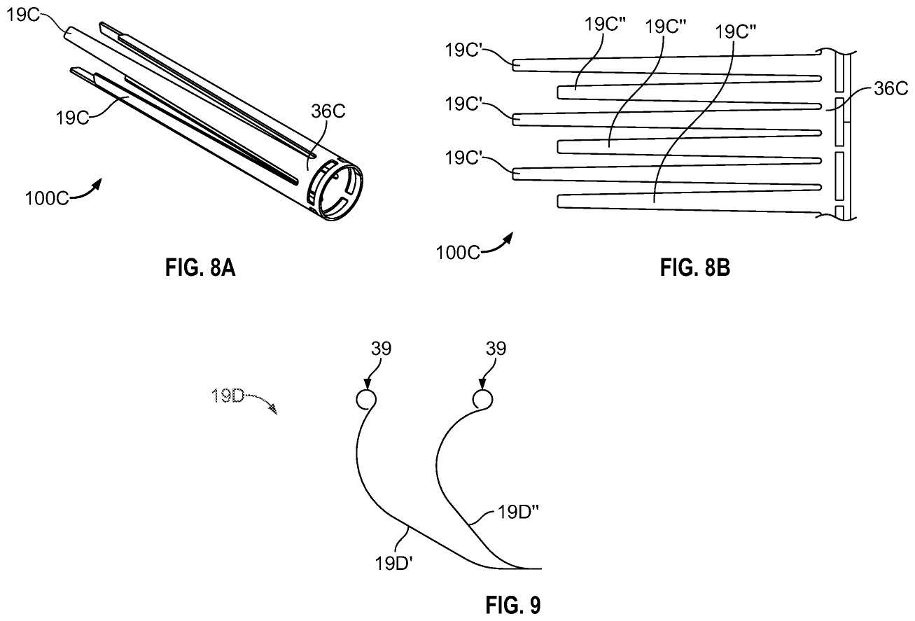

FIG. 8A is a schematic perspective view of a localization system 100C according to another embodiment. FIG. 8B is a schematic plan view of a laser cut design for the system 100C of FIG. 8A. Unless otherwise noted, the components of FIGS. 8A-8B may be the same as or generally similar to like-numbered components of FIGS. 1A-2H and 5A-7E, with some reference numbers appended by the letter “C.” In some embodiments, as shown in FIGS. 8A-8B, the plurality of struts 19C may differ in length. For example, as shown in FIGS. 8A-8B, the system 100C an include struts 19C arranged in a jester hat design. As shown, adjacent struts 19C may have different lengths. In some embodiments, every other strut may be designed to have approximately the same length. For example, as shown in FIGS. 8A-8B, first struts 19C′ of the plurality of struts 19C may have a first length, and second struts 19C″ of the plurality of struts 19C may have a second length 19C″ shorter than the first length. The second struts 19C″ may each be disposed circumferentially between the first struts 19C′. Although not illustrated in FIGS. 8A-8B, the struts 19C can include contact pads 24 at distal end portions thereof. In other embodiments, the struts 19C can include hooks 105 at distal end portions thereof.

Without being limited by theory, the different lengths may enable the system 100C to be supported against the vessel 37 at a plurality of longitudinal locations along the length of the vessel 37, which can improve localization and positioning. For example, in the expanded configuration of the struts 19C′, 19C″, the first struts 19C′ can engage with the vessel wall 37 at a location distal the location at which the second struts 19C″ engages with the vessel wall 37, such that the first and second struts 19C′, 19C″ engage with the vessel wall 37 at offset longitudinal positions. Engagement at offset longitudinal positions of the vessel wall 37 can beneficially improve stabilization of the pump 2 along multiple planes, and can also provide a resisting moment with multiple planes of contact. Moreover, the differing lengths of the struts 19C′, 19C″ can improve collapsibility of the struts by allowing the sheath 28 to separately engage the struts 19C′ and 19C″. For example, due to the differing lengths (and/or curvature) of the struts 19C′, 19C″, the sheath 28 may first engage a first set of struts (e.g., struts 19C″ in some embodiments) to cause the first set of struts to begin collapsing. During or after collapse of the first set of struts, the sheath 28 may subsequently engage a second set of struts (e.g., struts 19C′ in some embodiments) to cause the second set of struts to collapse. Dividing the collapse of the struts 19C′, 19C″ into two or more stages can beneficially reduce the amount of force used to collapse the respective struts 19C′, 19C″.

It should be appreciated that any of the support structures disclosed herein can comprise struts having different lengths. For example, in some embodiments, the plurality of struts (e.g., struts 19 or 19A) includes a first plurality of struts and a second plurality of struts. When the plurality of struts are in an expanded configuration, first contact elements (e.g., contact pads 24 or hooks 105) of the first plurality of struts can be configured to engage with the blood vessel wall at a first longitudinal position and second contact elements (e.g., contact pads 24 or hooks 105) of the second plurality of struts can be configured to engage with the blood vessel wall at a second longitudinal position that is spaced from the first longitudinal position. In some embodiments, the struts in the first plurality can have a different length from the struts in the second plurality. Additionally or alternatively, the struts in the first plurality can have a different radius of curvature (or departure angle) from the struts in the second plurality.

FIG. 9 is a schematic side view of a plurality of struts 19D according to various embodiments. In some embodiments, as shown in FIG. 9, a first set of struts 19D′ may have an elongate portion with a first radius of curvature, and a second set of struts 19D″ may have an elongate portion with a second radius of curvature different than (e.g., less than) the first. In the arrangement of FIG. 9, the first struts 19D′ have a steeper takeoff angle relative to the longitudinal axis L as compared with the second struts 19D″. An angle between a longitudinal axis of the pump 2 and of a portion of the second struts 19D″ adjacent to a base portion to which the struts are connected can be greater than a corresponding angle for the first struts 19D′, as shown in FIG. 9. The steeper takeoff angle of the first struts 19D′ may cause the sheath 28 to engage with and initiate collapse of the first struts 19D′ before engagement with the second struts 19D″. As explained above, staging, staggering or sequencing the collapse of the struts 19D′, 19D″ can beneficially reduce the force used to collapse the struts so as to improve operation of the pump 2. Staging, staggering, or sequencing the collapse of the struts can modulate the force profile over the length of motion of the sheath 28 over the struts 19 as felt from initial movement prior to collapsing, to the initial collapsing adjacent to the base 36, to final and full collapsing of the struts 19 by advancing the sheath adjacent to or beyond the distal ends of the struts. Staging, staggering, or sequencing can reduce the maximum force required over the length of motion of the sheath 28 over the struts 19. Moreover, the different curvature of the struts 19D′, 19D″ may also allow the distal ends of the struts 19D′, 19D″ to engage the vessel wall 37 at offset longitudinal positions, which, as explained above, can improve stabilization of the pump 2 due to, e.g., multiple planes or rings of contact with the vessel wall 37.

FIG. 9 thus illustrates embodiments where the struts 19D′, 19D″ may have approximately the same length along the longitudinal direction from proximal to distal ends in the retracted state but which may expand to contact a vessel wall at offset longitudinal positions, e.g., as may be defined by two spaced apart planes disposed transverse to, e.g., perpendicular to the longitudinal axis of the pump 2. The struts 19D′, 19D″, individually or in groups defining contact planes, can at least intermittently contact the vessel wall over a range of positions along the vessel wall that is two times, three times, four times, five times, six times, up to ten time, or up to one hundred times greater than the contact length of a contact pad or other vessel wall contact surface of the struts. It will be appreciated that dispersed contact areas of these sorts can also be provided by struts that have different lengths in the retracted state, as in FIGS. 8A-8B. In some embodiments, the contact element 104 at the second free end 39 of a strut 19D may be curled or coiled so that curled portion will contact the vessel wall 37. As a nonlimiting example, the second free end 39 of the strut 39D may be curled or coiled (e.g., at an angle in a range of approximately 270° to) 360°.

The contact area of the contact element 104 of a strut 19-19D may be designed so that endothelialization over longer durations does not impede or prevent removal of the device or increase the potential for trauma to the vessel wall 37 when the intravascular device (e.g., pump 2) is removed. In general, single-ended contact geometries can be pulled out more easily from under any endothelialization. In contrast, non-single ended contact geometries may increase the potential for trauma to the vessel wall 37 when the device is removed. In some embodiments with hooks 105, the strut 19B can be shaped so the action of advancing the sheath 28 to collapse the plurality of struts 19B will move the struts 19B in such a way as to pull the hooks 105 from the vessel wall 37 like a dart from a dartboard or in the opposite direction from which it was inserted. In some embodiments with contact pads 24, 24A, the pads 24, 24A may be tapered so they can be pulled out from under endothelialized tissue by translating the intravascular device (e.g., pump 2). Raising the edges of the contact pad 24, 24A (e.g., a “sled”-type design) may also discourage restrictive endothelialization.

The amount of radial force that presses the contact area at the second free end 39 of a strut 19-19D against the blood vessel wall 37 can be altered by varying the number of struts 19-19D, material of the struts 19-19D, and/or the geometry of the struts 19-19D and contact pads 24-24A. Important geometric factors may include, but are not limited to, the length of the strut 19-19D, cross-section of the strut 19-19D, attachment angle of the strut 19-19D to the pump housing 35, and curvature of the strut 19-19D. In general, a strut 19-19D will have a spring function, such that the more the strut 19-19D is compressed by the vessel wall 37, the higher the radial force of the strut 19-19D on the vessel wall 37. The design and shape forming of the strut may be selected to reduce this dependence so that the radial force provided by the strut 19-19D is relatively independent of the radius to which the strut is compressed. Equalization of such spring forces among a plurality of struts 19-19D can provide a centering positioning effect.

In some embodiments, a strut 19-19D may be designed for intermittent contact and have zero radial force unless it is in contact with the vessel wall 37. As a nonlimiting example, the plurality of struts 19-19D may have different lengths and/or geometries (e.g., FIGS. 8A-8B). The different lengths and/or geometries may arrange the struts 19C so that not all struts 19C touch the vessel wall 37 at the same time in some embodiments as shown in FIGS. 8A-8B. Further, is some example the struts 19-19D may be utilized with devices that exert forces on the struts 19-19D during operation (e.g., a gyroscopic effect), which may result in changes in forces exerted on the struts 19-19D. Because of the spring-like nature of the struts 19-19D, collapse or release in such situations can be facilitated. Note that each strut 19-19D in a plurality of struts may have a different geometry or contact region design.

In some embodiments, the struts 19-19D can have knees 102 as explained above. A knee 102 in a strut may function to keep part of the strut 19A-19D away from the inner wall of the sheath 28 when the plurality of struts 19A-19D are collapsed within the sheath 28. For example, the knee 102 may function to keep a hook 105 away from the inner wall of the sheath 28 so that the hook 105 does not contact the sheath 28 and create particulates through abrasion, cutting, or gouging. The knee 102 can comprise an inflection zone disposed between the first end 38 and the second end 39, the second end 39 resiliently deflectable toward and away from the longitudinal axis L of the pump housing 35. A frec state of the strut can space the second end 39 thereof away from the longitudinal axis L of the pump housing 35. The second end 39 of the strut can be configured to engage the blood vessel wall 37 (e.g., to at least intermittently contact the vessel wall 37). The inflection zone can comprise an S-connection between a first span of the strut and a second span of the strut. The first span and the second span can be disposed along parallel trajectories.

Minimizing the diameter of the sheath 28 used to implant or retrieve an intravascular device (such as the pump 2) can be important. An advantage of the embodiments disclosed herein is that the plurality of struts 19-19D can be collapsed to a diameter equal to or smaller than the diameter of the pump 2 itself so that a large sheath is not required due to the presence of the plurality of struts 19-19D.

In some embodiments, a plurality of struts 19-19D may be designed to contact the vessel wall 37 in multiple transverse planes (for example, at multiple longitudinal positions) along the central axis of the vessel. In some embodiments, a plurality of struts 19-19D may be attached to the pump 2 in one transverse plane, but the struts 19-19D can have different geometries and can contact the vessel wall 37 in multiple transverse planes along the central axis of the vessel. In some embodiments the plurality of struts 19-19D may be attached to the pump 2 in more than one transverse plane along the central or longitudinal axis L of the pump 2. As a nonlimiting example, there may be a set of struts 19-19D at cach end of the pump 2 (e.g., at proximal and distal ends of the pump 2).

In some embodiments, a plurality of struts 19-19D may be directly integrated into the pump 2 such that the shroud 16 and struts 19-19D are monolithically formed in a single piece. In other embodiments, the plurality of struts 19-19D may be coupled or connected to the pump 2 instead and may comprise one or more separate piece(s). As a nonlimiting example, the struts 19-19D may be attached a ring that is attached to the pump 2.

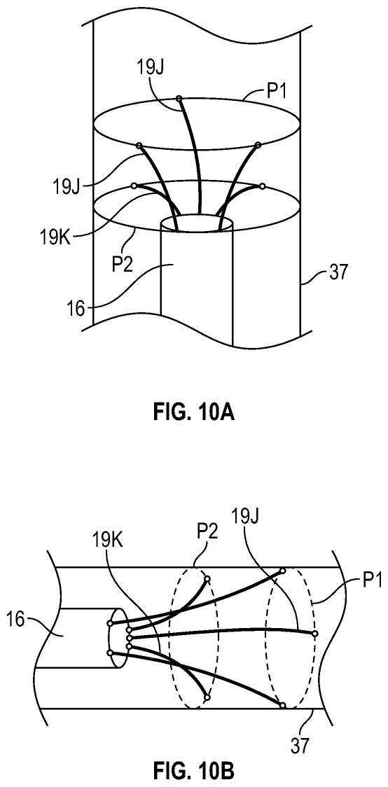

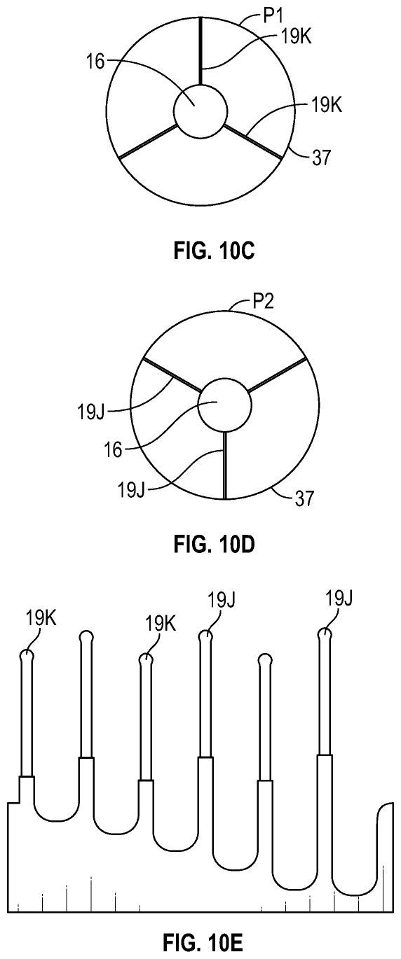

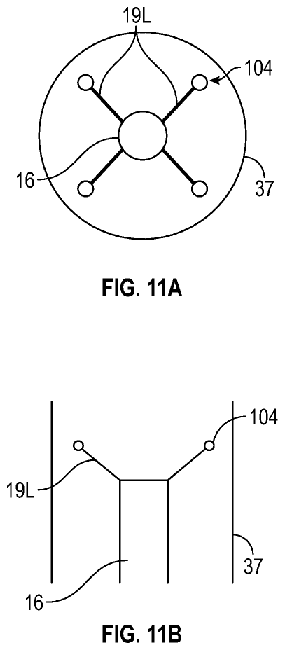

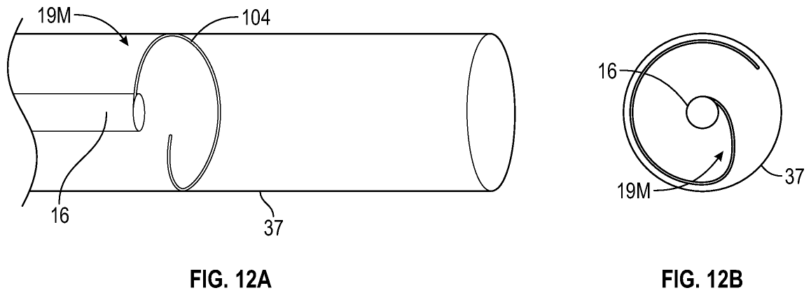

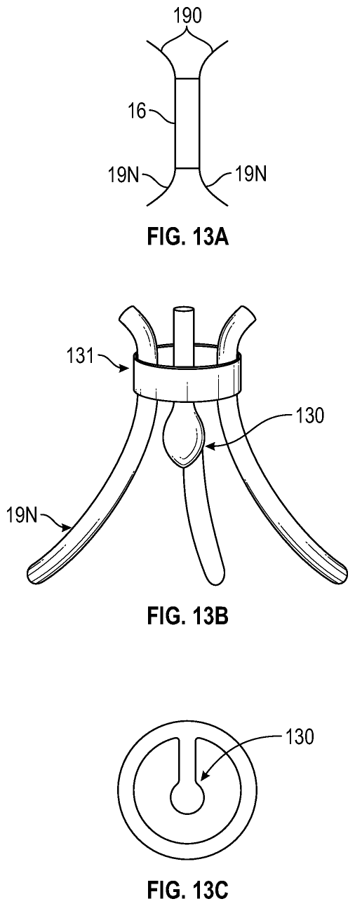

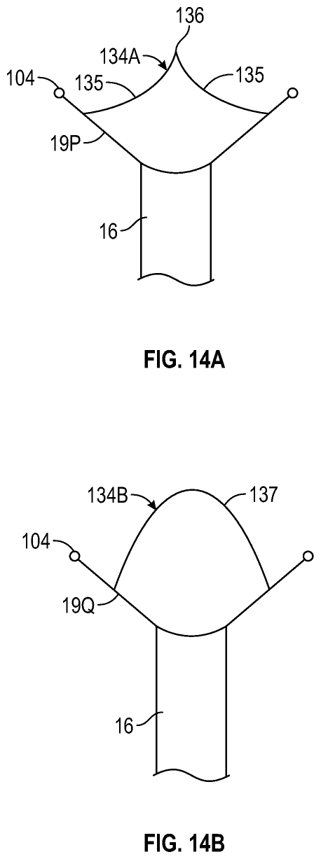

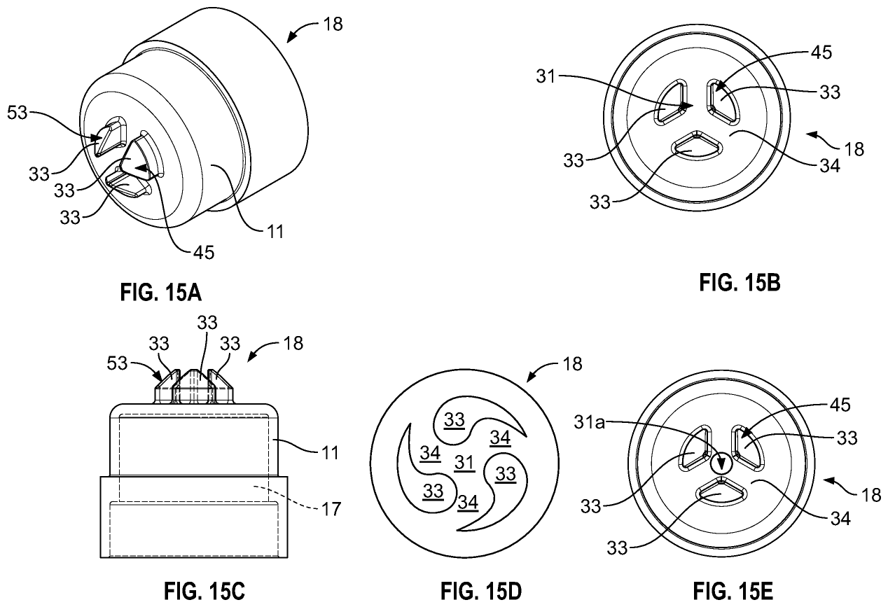

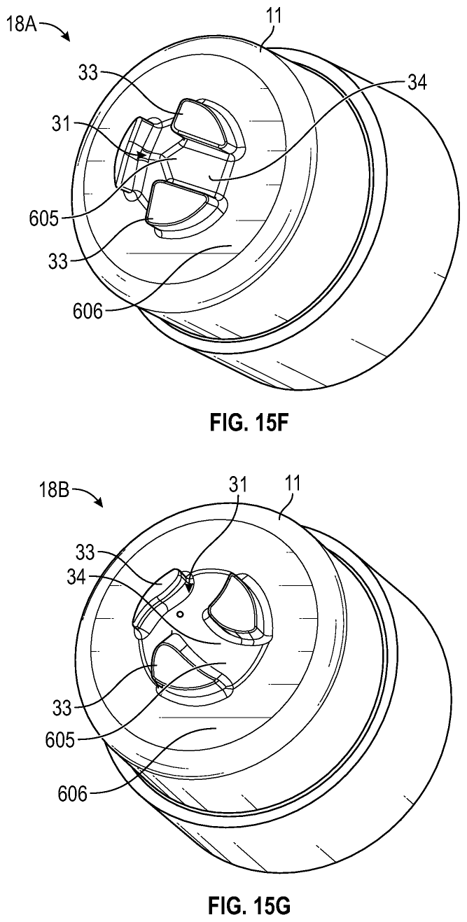

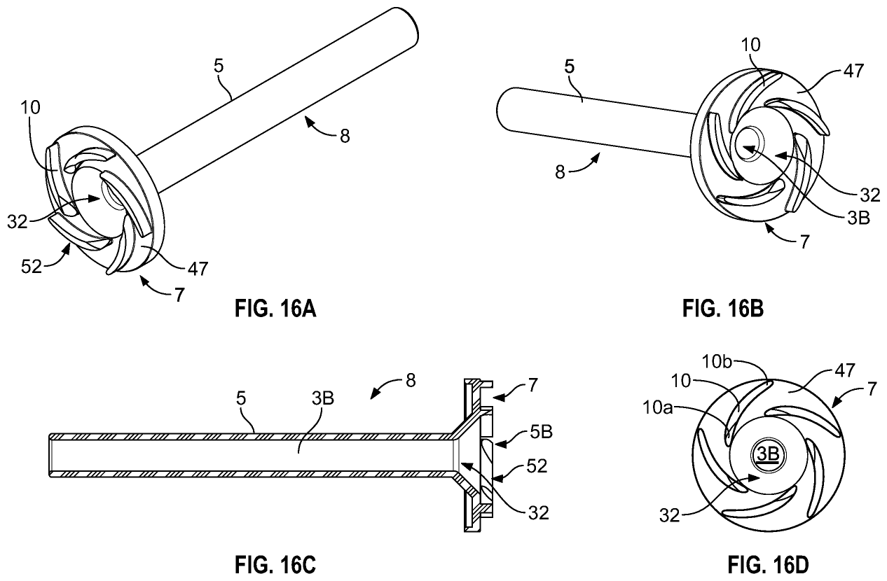

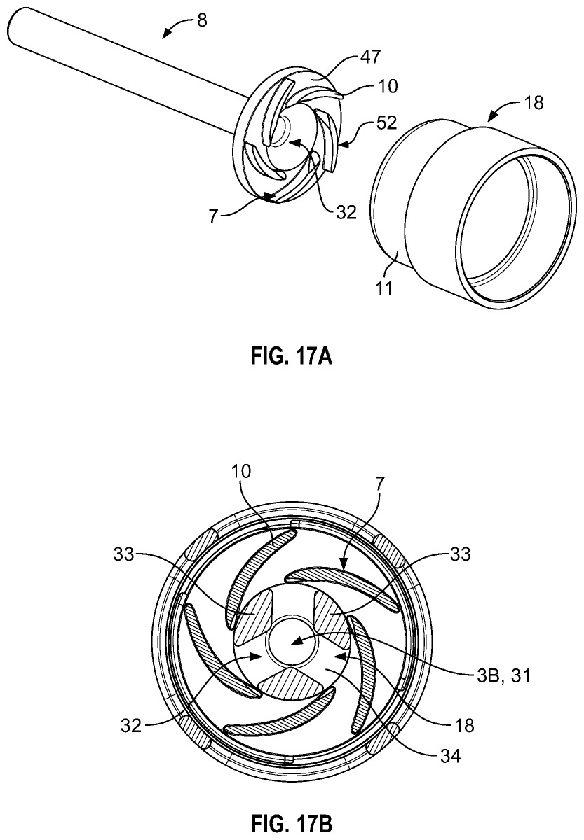

FIGS. 10A-10D illustrate an example of a first plurality of struts 19J extending from the shroud 16 and a second plurality of struts 19K extending from the shroud 16. The struts 19J, 19K can extend distally and radially outwardly in the expanded configuration. As shown in FIGS. 10A-10D the first struts 19J can at least intermittently contact a vessel wall 37 at a first plane P1, and the second struts 19K can at least intermittently contact the vessel wall 37 at a second plane P2. The first plane P1 can be disposed distal the second plane P2. By having contact at multiple planes P1, P2, the struts 19J, 19K can provide improved stabilization and/or localization at multiple locations. FIG. 10E illustrates a two-dimensional pattern of the struts 19J, 19K shown in FIGS. 10A-10D.

FIGS. 11A-11B illustrate a plurality of struts 19L extending from the shroud 16, according to another embodiment. Each strut can extend radially and distally outward from the pump housing or shroud 16. Each strut 19L can have a contact element 104 at a distal end thereof. The contact element can be configured to at least intermittently contact a wall 37 of a blood vessel. As explained herein, the struts 19L can have an expanded diameter in an expanded configuration. As illustrated in FIGS. 11A-11B, the expanded diameter of the struts 19L can be less than a diameter of the blood vessel 37. During operation of the pump 2, the contact elements 104 of the struts 19L can serve as bumpers to maintain the pump 2 in a generally central location within the vessel 37.

FIGS. 12A-12B illustrates a support structure comprising a strut 19M shaped in a curved or coiled shape. The strut 19M can have a contact element 104 at a distal end thereof. The contact element 104 can be configured to at least intermittently contact a wall 37 of a blood vessel. As shown, the strut 19M can be at least partially revolved about the longitudinal axis of the pump. For example, the strut 19M can be disposed about the longitudinal axis in a helical profile. In various embodiments, the strut 19M comprises a coiled spring. The coiled spring structure of the strut 19M can resiliently and at least intermittently engage with the vessel wall 37 and can serve as a bumper to maintain the pump 2 in a generally central location with the vessel. The strut 19M can comprise a shape memory metal alloy, such as nitinol, in various embodiments. The strut 19M can comprise other suitable materials as well.

FIGS. 13A-13C illustrate a pump having a shroud 16 with a first plurality of struts 19N extending proximally from the shroud 16 and a second plurality of struts 19O extending distally from the shroud 16. The struts 19N, 19O can be shaped in any suitable manner disclosed herein. Having struts 19N, 19O at opposing ends of the shroud 16 can provide a redundant mechanism and/or improved control for stabilizing and/or localizing the pump within the blood vessel. However, with the proximally-extending struts 19N, it may be challenging to collapse the struts 19N with the sheath 28, as the sheath 28 may catch on the struts 19N. Accordingly, a slip ring 131 can be provided over the struts 19N at or near the shroud 16. To collapse the struts 19N, the clinician can insert a retrieval device to grab a snare feature 130. The clinician can move the snare feature 130 proximally to pull the struts 19N together an collapse the struts 19N.

FIGS. 14A-14B illustrate struts 19P, 19Q having a brace 134A, 134B extending between the struts 19P, 19Q. Each strut 19P, 19Q can have a contact element 104 at a distal end thereof. The contact element 104 can be configured to at least intermittently contact a wall 37 of a blood vessel. Beneficially, the brace 134A or 134B can provide improved radial resistance for the support structures. The braces 134A, 134B can extend between and mechanically connect to the two adjacent struts 19P or 19Q.

In FIG. 14A, the brace 134A can include a multi-segment brace. For example, the brace 134A can include a first distally-extending segment 135 extending from the first strut 19P and a second distally-extending segment 135 extending from the second strut 19P. The first and second distally-extending segments 135 can be joined at a connection location 136. By contrast, in FIG. 14B, the brace 134B can comprise an arch brace having a curved profile 137 extending between the first and second struts 19Q. The braces 134A, 134B can improve the radial support strength of the struts 19P, 19Q.

### Tether

In some embodiments, one or more tethers may be a component of the localization and positioning system 100-100C. Devices, such as the pump 2, that utilize a cable or lead for power or infusion can use that cable or lead as a tether. For example, as shown herein, the power lead 20, 20A can serves as the tether in the illustrated embodiments. The tether (e.g., power lead 20, 20A) can have an anchor point outside the blood vessel and/or the patient, and can limit translation of the intravascular device (e.g. away from that anchor point). As explained herein, for example, the connector 23 at the proximal end 21 of the system 1 can connect to a console (which can serve as the anchor point in some embodiments) outside of the patient's body. In some embodiments, the arteriotomy and path through the skin of the patient can serve as the anchor point for the tether. Sutures may be used to anchor the tether (e.g., power lead 20, 20A) adjacent to the proximal end 21 in some procedures.

### Propulsion

One nonlimiting example of intravascular devices that may be used with the disclosed embodiments is the blood pump 2A, as shown in, e.g., FIGS. 7A-7E. As shown in FIG. 7A, and as explained above, the sheath 28 can be inserted percutaneously to a treatment location in a blood vessel, such as the descending aorta. In some embodiments, as shown in FIG. 7B, after placement of the sheath 28, the pump 2A can be pushed distally within the sheath 28 by way of a stiffening member or guidewire (not shown) that can be disposed within the central lumen 55. In other embodiments, the pump 2A can be pre-loaded in the sheath 28, and the sheath 28 and pump 2A can be advanced together to the treatment location. As shown in FIGS. 7C-7D, relative motion can be provided between the sheath 28 and the pump 2A to urge the pump 2A out of the sheath 28. The support structure including the struts 19-19D can self-expand and contact the inner wall of the vessel 37. The struts used in the support structure of the pump 2A shown in FIGS. 7A-7E can include any of the struts 19-19D described herein. For example, in some embodiments, such as that shown in FIG. 7C, a mesh 147 can extend or span between adjacent struts at a location near the distal end of the shroud 16. The mesh 147 can extend partially along length(s) of the struts, e.g., within a range of 10% to 70% of a length of the strut(s). The struts 19A of FIG. 7D are shown with the contact pads 24. The struts of FIG. 7E are shown with the hooks 105.

Once the struts are deployed, the impeller 6 can be activated to pump blood. Some blood pumps 2A discharge blood in jets 34 or exert significant forces during operation. These pumps 2A may generate a reaction (or propulsive) force 133 on the pump 2A in the opposite direction of the pump discharge, e.g. when pumping down a propulsive force 133 may result upwardly as shown in FIG. 7D. Some embodiments may be designed to take advantage of this propulsive force 133 as a component of the localization system 100-100C. As a nonlimiting example, the struts 19-19D may provide a geometry that causes an increase in the spring-like forces as a result of the propulsive force 133, e.g., the propulsive force 133 may further compress the struts 19-19D and increase the spring force. In various embodiments, a longitudinal component of the thrust force 133 along the longitudinal axis L can be opposed by tension in the tether (e.g., the power lead 20, 20A). A transverse component of the thrust force 133 directed transverse to the longitudinal axis L (e.g., along the radial axis R) can be opposed by strain energy stored in at least one of the elongate struts 19-19D upon deflection of the strut(s) 19-19D. As explained herein, when the procedure is complete, the clinician can provide further relative motion between the sheath 28 and the pump 2A to collapse the struts 19-19D into the sheath 28 (see FIG. 1H).

Beneficially, in various embodiments disclosed herein, the power lead 20, 20A can serve as a tether that is sufficiently strong so as to oppose loads applied in opposite directions at opposite ends thereof. In some pumps, the thrust from the pump 2 may be too strong such that, if the proximal end of the tether is not sufficiently anchored and/or if the power lead 20, 20A is not sufficiently strong, the pump 2 can move through the blood vessel. In such a situation, the pump 2 may stretch the tether, and/or the tether may not be sufficiently anchored. Beneficially, the embodiments disclosed herein can utilize the elongate hollow member and conductor wires which can be sufficiently strong such that, when anchored outside the blood vessel, a longitudinal component of the thrust force generated by the impeller directed along the longitudinal axis of the pump can be adequately opposed by the tether. Thus, in various embodiments, the tether (e.g., power lead 20, 20A) can be configured to maintain a position of the pump 2 within the blood vessel without requiring contact between the pump 2 and a blood vessel wall 37 of the blood vessel.

In some embodiments, the struts of the support structure need not contact the wall 37 during operation of the blood pump 2, and the tether can serve to adequately position the pump 2. In some procedures, the strut(s) may at least intermittently contact the blood vessel wall 37 (e.g., the struts may only intermittently contact the wall 37). In such arrangements, the strut(s) may intermittently come into contact with the wall 37 and move away from the vessel wall 37 throughout the procedure. Accordingly, the embodiments disclosed herein need not require constant contact between the support structure of the pump and the vessel wall 37. Indeed, in such embodiments, the struts may comprise short and/or stubby struts that may serve as bumpers that atraumatically, e.g., resiliently, engage with the vessel wall 37 intermittently as the pump 2 moves towards the wall 37, and pushes the pump 2 back towards a central location of the vessel. In some embodiments, the struts may be omitted such that the tether and thrust force establish the position of the pump in operation. In other embodiments, however, the struts may be shaped or configured to maintain substantially constant contact with the vessel wall 37 when in the deployed configuration during use of the pump 2. In still other embodiments, the pump 2 may not include struts, such that the tether may serve the positioning and/or localization function without struts.

### Example Designs

The various design features discussed above may be mixed and combined in any fashion desired. Nonlimiting examples described herein below illustrate one possible embodiment that combines the design elements described above and are not an indication of the bounds of potential combinations.

The systems and methods discussed herein are used to provide localization and positioning of a device, such as an intravascular pump 2, 2A. A plurality of struts 19-19D with contact elements 104 project out from a ring attached to the inlet end of the pump 2. The embodiments of FIGS. 1A-1H and 6A-3G show four struts 19-19B, but any number of struts may be used. For example, as shown in FIGS. 8A-8B, in some embodiments more than four struts (e.g., six struts 19C) can be used. The contact pads 24, 24A are shown as circular, but any shaped contact pads 24, 24A may be used. The strut geometry is designed to provide radial force within a set range at the strut contact pads 24, 24A for vessels within a certain diameter range. The struts 19-19D can also be designed to reduce or minimize the force required for the sheath 28 to collapse the struts 19-19D.

The circular contact pads 24, 24A can be designed to slide on the inner artery wall 37 rather than cause any trauma. With this tuning of the radial force, the plurality of expanded struts 19-19D provides consistent positioning of the inlet port 27-27B of the pump 2, 2a in the center of the vessel lumen and resists, but does not strictly prevent, translation and rotation of the pump 2, 2a. This feature allows safe translation of the pump 2, 2a whether intentional (to move the pump 2, 2A to a preferred location) or unintentional (e.g., if the power lead is yanked).

Providing limited localization is sufficient because in some embodiments the propulsive force 133 of the pump 2, 2A tends to move it in a superior direction, and/or this movement may be limited by the tether effect of the pump's power lead 20, 20A. One advantage of this embodiment is providing stable long-term localization, while allowing instantaneous movement of the pump 2, 2A with minimal or reduced risk of trauma to the vessel wall 37. This embodiment, for example, is compatible with a greater freedom-of-motion for the patient who is free to sit up, bend at the waist, and/or make other similar motions.

In some embodiments, the strut geometry may be altered so that the struts 19-19D only make intermittent contact with the vessel wall 37. In such an embodiment, the propulsive force 133 acting against the tether (e.g., power lead 20, 20A) provides localization and the struts 19-19D maintain positioning of the port 27-27B of the pump 2, 2A in the center of the lumen of the vessel.

### Advantages

The systems and methods discussed herein, including without limitation the embodiment described in detail and illustrated in the drawings, has a number of advantages. Many of these advantages are described above. The following are only additional non-limiting examples of advantages, some of which arise from the combination of various design elements.
a. Struts 19-19D (including struts 19C′, 19C″, 19D′, 19D″) designed to not increase the diameter of the pump 2 when the struts 19-19D are in the collapsed configuration.
b. Struts 19-19D (including struts 19C′, 19C″, 19D′, 19D″) with knees 102 and hooks 105, such that the knees 102 prevent the hooks 105 from contacting the inner surface of the sheath 28 during implantation or retrieval of the pump 2.
c. Atraumatic contact pads 24, 24A designed to resist, but not eliminate translation or rotation of the intravascular device (e.g., pump 2, 2A) that
   i. Work in conjunction with a tether (e.g., power lead 20, 20A) and propulsive force 133; and/or
   ii. Become more resistant to translation over time due to desired endothelialization.
d. Intermittent contact positioning (centering) with struts 19-19D (including struts 19C′, 19C″, 19D′, 19D″) with long-term localization effected by the propulsive force 133 working against a tether (e.g., power lead 20, 20A).

### V. Examples of Segmented Cone Bearings

As shown in FIGS. 1A and 1C, the drive unit 9 can comprise a drive magnet 17 and a drive bearing 18 between the drive magnet 17 and the impeller assembly 4. The drive bearing 18 can provide a magnetic coupling and a fluid bearing interface between the drive magnet 17 and a rotor assembly that comprises the driven or rotor magnet (not shown) and an integrated rotor core that includes the impeller shaft 5 and a secondary impeller 7. In various embodiments, the drive bearing 18 can comprise a segmented cone bearing. Cone bearings can comprise a convex (e.g., generally conical) shaped member 45 seated inside a generally concave (e.g., conical) opening 32 or cavity of the rotor assembly 46. The concave opening 32 can serve as a concave bearing surface sized and shaped to mate with the convex member 45. The concave opening 32 can comprise an angled concave cavity sized to receive the convex member 45. The drive unit 9 can comprise a convex member sized to fit within the angled cavity of the concave opening 32.

The bearing interface region of this bearing design can be formed by the matching surfaces of the conical or convex member 45 and the conical or concave opening 32 and the space between them. A cone bearing can provide both axial and radial confinement. The axial confinement from a single cone bearing can be in one direction only. Cone bearings with steep slopes provide relatively more radial confinement, and cone bearings with shallower slopes provide relatively more axial confinement. In some embodiments, the conical shaped member 45 can be modified to reduce hemolysis and/or clotting. In some embodiments, the conical member 45 can be truncated by a cylinder coaxial to the axis of the cone (or axis of rotation) to remove base portions of the cone. In some embodiments, the conical member 45 can be truncated by a plane perpendicular to the axis of the cone (creating a frustrum or a frustoconical surface). In other embodiments, the conical member 45 can be truncated by both a cylinder and a cone. In some embodiments, the surface of the conical opening 32 may be modified in a similar manner in conjunction with the conical member 45 or instead of the conical member 45. One or the other or both of the surfaces of the conical member 45 and conical opening 32 may also be modified by holes, gaps, channels, grooves, bumps, ridges, and/or projections. Each of the surfaces of the conical member 45 and conical opening 32 may also be formed as part of other components of the pump with any overall shape.

Given the general possibility of holes, grooves, channels, or gaps in either the conical member 45 and/or conical opening 32, either of their surfaces comprise of a plurality of separate bearing surfaces in the plane of the generally conical shape defining the member 45 or opening 32. In such a manner the opening 32 and/or the conical member 45 of the bearing pair may be formed by a plurality of separate surfaces or a segmented surface. The plurality of separate surfaces or the segmented surface that make up either the conical member 45 or conical opening 32 of the bearing pair may extend from the same component or part, or may extend from distinct components or parts. Grooves and gaps in either the conical member 45 and/or conical opening 32 may be created by removing material from a single generally conical surface or by using a plurality of separate surfaces.

In some embodiments of a modified cone bearing, the conical member 45 of the bearing pair can comprise a convex bearing surface having a segmented frustoconical shape formed from a plurality of distally-extending segments 33 (FIGS. 15A-15D). The distally-extending segments 33 can extend distally from the drive unit cover 11. The segments 33 can be spaced apart circumferentially to define at least one channel 34 between adjacent segments 33. Three segments 33 are shown in FIGS. 15A-15D, but any suitable number of segments 33 may be utilized. As shown, the segments 33 can be separate components arising from a common part with gaps or channels 34 between them, but the segments 33 may also be separated by shallow or deep grooves. The gaps, grooves or channels 34 may follow any path. In the illustrated embodiment, the channel(s) 34 extend radially outward from a central recess or hollow 31 (also referred to herein as a void) at a location proximal a proximal end portion 5B of the impeller shaft 5. In some embodiments, the width and depth of any groove or channel 34 may vary along its path. In some embodiments, two or more channels 34 may join or separate. In certain embodiments, two or more channels 34 may join to form the central hollow area 31 coaxial with the axis of the conical surfaces and/or with the longitudinal axis of rotation L. In some embodiments, the conical opening 32 of the bearing pair can be a continuous (e.g., no gaps, channels, or grooves), generally conical surface. The relative angles of the cone bearings (e.g., the segments 33) and spacing between segments 33 can be selected to provide a desired flow profile through the channel(s) 34 described herein. For example, increased spacing between the segments 33 can provide increased flow through the channels 34. Together, the segmented conical member 45 of the drive bearing 18 with channels 34 between the segments 33 and the continuous conical opening 32 can serve as a “segmented cone bearing”.

The channels 34 between the segments 33 allow interrupted contact between bearing surfaces. This interrupted contact provides, without limitation, benefits for reduced hemolysis. For example, in embodiments in which the conical opening 32 is part of the rotating member (e.g., the impeller shaft 5), the channels 34 between the segments 33 can ensure that at least one point throughout the length or height of the conical opening 32 on the rotating member 5 is intermittently exposed by the conical opening 32 and not continuously covered by the bearing pair. This design promotes exchange of a lubricating layer blood over the entire bearing interface. The channels 34 also generate pressure changes that contribute to lubricating layer formation and dispersal as described above for the sleeve bearing 15, 15A, 15B.

In some embodiments, additional features may promote blood flow through the central hollow 31 and channels 34 of the segmented cone bearing. In some embodiments blood may flow in through the channels 34 and exit via the central hollow 31. In other embodiments blood may flow into the central hollow 31 (e.g., from the secondary flow pathway 3B of the impeller shaft 5) and exit via the channels 34. This net flow of blood through the central hollow 31 and channels 34 may serve to ensure the volume of blood in the channels 34 and central hollow 31 is constantly flowing to provide a source of fresh blood for lubricating layer exchange, to carry away heat, and/or to reduce the time that blood is exposed to conditions within the bearing region that may increase the potential for hemolysis or thrombus formation. Accordingly, in various embodiments, a concave bearing surface (which can comprise or be defined by the concave opening 32) can include a fluid port to deliver blood proximally along the second flow pathway 3B. The convex bearing surface (which can comprise the convex member 45) can including a void (e.g., the central hollow 31), which can be disposed on the longitudinal axis L. The one or more channels 34 can extend radially outward from the void or central hollow 31. The void can be in fluid communication with the fluid port (e.g., an interface between the flow tube 5 and the conical opening 32) so as to direct blood radially outward along at least one channel 34.

As shown in FIG. 17A, the segments 33 of the convex member 45 can be shaped to fit within the concave bearing surface comprising the concave opening 32. In some embodiments, as shown in FIGS. 16A-17B, a direct secondary flow pathway 3B (for example through the flow tube of the impeller shaft 5 shown in FIGS. 16B-16D and 17B) may provide proximally-flowing blood into the central hollow 31. In some embodiments a secondary or second impeller 7 may be used to drive the secondary flow of blood through the bearing region, e.g., through the second flow pathway 3B, the central hollow 32, and radially outwardly through the channel(s) 34. The primary impeller 6 of the pump and/or the additional secondary impeller 7 may assist in drawing the blood proximally and directing the blood radially outwardly along the channel(s) 34. FIGS. 16A-16D show the secondary impeller 7 that draws blood out through the channels 34 of the segmented cone bearing. As explained herein, the secondary impeller 7 and impeller shaft 5 can form an integrated rotor core. The secondary impeller 7 can have a plurality of vanes 10 as explained herein to assist in directing blood radially outward through the channel(s) 34 of the drive bearing 18.

Keeping the segmented cone bearing elements or segments 33 near the central longitudinal axis L of the pump can have several advantages. For example, in the illustrated embodiment, the bearing elements 33 can be more directly exposed to the blood flow from the flow tube of the impeller shaft 5 along the second flow pathway 3B. Further, the bearing elements 33 can have a smaller radius where the linear speed of the rotating member is lower. Placing the bearing elements or segments 33 near the axis L of the pump allows the vanes 10 of the secondary impeller 7 to be placed at a greater radius where the linear speed of the rotating member or shaft 5 is higher.

FIG. 15D shows an embodiment in which the channels 34 between the segments 33 follow a curved path from the central hollow 31. The channels 34 can be configured to increase flow and reduce shear forces on the blood. In some embodiments, the depth of the channels 34 may be varied to form a central flow diverter 31a as shown in, e.g., FIG. 15E. The flow diverter 31a may comprise a distally-extending projection (e.g., a cylindrical projection, a conical projection, a pyramidal projection, etc.) disposed in a central region of the bearing between the segments 33. In the illustrated embodiment, the flow diverter 31a can comprise a symmetrical flow diverter. The flow diverter 31a may aid blood coming from the flow tube or lumen of the shaft 5 to transition from axial flow to radial flow to exit through the channels 34. The flow diverter may optionally be manufactured as one or more separate pieces that are then attached in the central hollow 31 and/or channels 34. In some embodiments, the flow diverter 31a may comprise a generally right cylindrical shape extending distally from the bearing 18. In other embodiments, the flow diverter 31a can have a tapered, for example, conical, profile.

The interface between the segments 33 of the conical member 45 and concave, e.g., conical, opening 32 of the segmented cone bearing can be lubricated by blood. Depending on geometry, materials used, and operating conditions, this lubrication may be hydrodynamic lubrication, elastohydrodynamic lubrication, boundary lubrication, or mixed lubrication. The channels 34 between the segments 33 of the conical member 45 of the bearing pair may promote fluid exchange so that a portion of the blood that makes up the lubricating layer between a region of the conical opening 32 of the bearing pair over one segment 33 of the conical member of the bearing pair is replaced by fresh blood in the lubricating layer that forms between that same region of the conical opening 32 of the bearing pair and the next segment 33 of the conical member of the bearing pair during rotation. The width and depth of the channels 34 can be altered to encourage this exchange. In various embodiments, the height and lateral spacing of the segments 33 can be selected to provide a desired channel depth and width. For example, a width of the channels 34 can be in a range of 0.02″ to 0.06″, in a range of 0.03″ to 0.05″, or in a range of 0.035″ to 0.045″ (for example, about 0.04″ in some embodiments). The surfaces of the segments 33 of conical member of the bearing pair along the channels 34 form the leading and trailing edges (as seen by a region of the conical opening 32 of the bearing pair) of the segments 33 of the conical member of the bearing pair. The distance of the leading and trailing edges from the conical opening 32 may also be modified to encourage fluid exchange. For example, the edges may be beveled or rounded or the distance of the leading and trailing edges may taper away or towards the surface of the conical opening 32.

The surfaces of the segments 33 of the conical member 45 of the bearing pair may also be modified to diverge from a perfect conical surface to promote formation of a lubricating layer. For example, one or more surfaces of the segments 33 of the conical member 45 of the bearing pair may be shaped so the normal distance to the surface of the conical opening 32 of the bearing pair decreases from the leading edge to the trailing edge. Such a surface contour may encourage creation of fluid wedges between the segments 33 of the conical member 45 and the conical opening 32 of the bearing pair for improved lubrication. In another embodiment, the surfaces of the segments 33 of the conical member 45 and conical opening 32 of the bearing pair may be smooth and well matched to allow a relatively thin lubricating layer of relatively uniform thickness to form. It should be appreciated that although conical member 45 and conical opening 32 are described as having a generally conical shape in some embodiments, the member 45 and opening 32 may generally be considered convex member 45 and concave opening 32. The shapes of the convex member and the concave opening 32 may be any suitable mating shapes.

The flow of blood driven by the secondary impeller 7 from the central hollow 31 through the channels 34 provides fresh blood for exchange of the lubricating layers and carries away heat in the bearing region. Both functions are important to reducing the potential for thrombus formation in the segmented cone bearing.

The segments 33 of the conical member 45 of the bearing pair and the conical opening 32 of the bearing pair may each be made of any suitable blood compatible bearing material. As a non-limiting example, the segments 33 of the conical member of the bearing pair may be made out of titanium or stainless steel and/or the conical opening 32 of the bearing pair may be made out of PEEK or polyethylene.

By making one side of the bearing pair relatively hard and the other side of the bearing pair relatively soft, the bearing pair may initially undergo boundary or mixed lubrication where surface asperities are worn to the point where the surfaces of the conical member and conical opening are smooth and well-matched enough for hydrodynamic or elastohydrodynamic lubrication to dominate. Having one side of the bearing pair be relatively softer may increase the range over which elastohydrodynamic lubrication is present. In some embodiments, the continuous, conical opening 32 of the bearing pair will be softer and the segmented, conical member of the bearing pair will be harder. This arrangement may help preserve special geometric features of the segments 33 on the conical member of the bearing pair. In some embodiments, the continuous, conical opening 32 of the bearing pair will be harder and the segmented, conical member 45 of the bearing pair will be softer. This arrangement may help preserve the surface of the opening 32 as a surface of rotation about the longitudinal axis L. In other variations the conical opening 32 and the conical member 45 can be of similar or even the same hardness which can provide the advantage of dimensional and shape stability throughout the operation of the pump 2.

In cases where hydrodynamic lubrication dominates, the normal distance between the segments 33 on the conical member of the bearing pair and the conical opening 32 of the bearing pair may be small enough to exclude red blood cells. In these cases, exchange of the lubricating layer may be less important as long as heat is still transferred away. Given sufficient exclusion of red blood cells, a continuous (e.g., without channels or grooves) conical member 45 of the bearing pair may still demonstrate low potential for thrombus formation as long as heat can be transferred away quickly enough. In some embodiments, this may be accomplished by eliminating or covering the channels 34 to form a continuous conical surface. Blood flow through the covered channels 34 may transfer sufficient heat from the bearing pair.

The segmented bearing embodiments described above provide an additional advantage of enhancing the flexibility of the portion of the pump 2 in the vicinity of the pump head 50. The impeller assembly 4 can be coupled with the drive unit 9 in a manner that permits some motion between the impeller assembly 4 and the cover 11. For example, the pump 2 may be delivered through tortuous or curving vasculature or may be inserted from outside the patient to inside a blood vessel in tight bends. The impeller assembly 4 can tip toward one or more of the segments 33 and away from one or more segments at the conical opening 32 such that proximal end face of the impeller assembly is at a non-parallel angle to the distal face of the cover 11. The motion may be significant compared to a mounting of the impeller assembly 4 on a shaft rotatably supported in a drive unit. The tipping of the impeller assembly 4 can occur with a flexing of the shroud 16, which may be flexed in high bending stress maneuvers. In some embodiments, the shroud 16 is made of an elastic material, such as nitinol, such that the pump head 50 can flex and elastically return to an undeflected state without elongation.

The secondary impeller 7 can be disposed proximal the impeller 6. In some embodiments, as shown in FIGS. 16A-17B, the secondary impeller 7 can comprise a flange 47 extending non-parallel (e.g., radially outward along the radial axis R) from the proximal end portion 5B of the impeller shaft 5 and a plurality of vanes 10 on a proximally-facing surface of the flange 47. The flange 47 can extend non-parallel and radially outward from the impeller shaft 5. In some embodiments, the flange 47 may not extend radially beyond the shroud 16. In some embodiments, the flange 47 may not extend radially beyond an adjacent portion of the impeller assembly 4, e.g., may not extend radially beyond an integrated streamlined fairing 13, discussed below. In some of these embodiments, the flange 47 can comprise a section of the combined rotor surface that lies in a plane perpendicular to the longitudinal axis L. As shown in FIGS. 16A-16C and 17A, the vanes 10 can extend proximally from the flange 47 and can have a curved profile circumferentially about the longitudinal axis L. The vanes 10 can be disposed in the space between the proximal face of the flange 47 and the distal end of the drive unit 9. The concave opening 32 can comprise an angled cavity extending inwardly and distally relative to the generally proximally-facing surface of the flange 47. The rotor magnet 12 can be disposed adjacent a distally-facing surface of the flange 47. Each of the vanes 10 can have an inner end 10a disposed at or near the concave opening 32 and an outer end 10b extending radially and circumferentially outward from the inner end 10a along the flange 47. The flange 47 can be coupled to or formed with the proximal end of the impeller shaft 5. In some embodiments, for example, the flange 47 can be monolithically formed with (e.g., seamlessly formed with) the impeller shaft 5. In other embodiments, the flange 47 and impeller shaft 5 can be separate components that are mechanically connected to one another (e.g., welded or otherwise coupled). In some embodiments, the vanes 10 can be monolithically formed with the proximally-facing surface of the flange 47. In other embodiments, the vanes 10 can be mechanically connected to the proximally-facing surface of the flange 47.

As shown in FIG. 16D, the vanes 10 can extend circumferentially about the longitudinal axis L in a manner such that adjacent vanes 10 circumferentially overlap. For example, the radially outer end 10b of one vane can circumferentially overlap with, and be disposed radially outward from, the radially inner end 10a of an adjacent vane. The vanes 10 can be prevented from contacting the drive unit 9 by the thrust bearing aspect of the segmented cone bearing. As the impeller assembly 4 rotates, the vanes 10 can pump blood radially out of the channels 34 in the segmented cone bearing and thereby increase net flow through the flow tube of the impeller shaft 5 and segmented cone bearing. As shown, blood can exit the flow tube of the impeller shaft 5 at a location proximal the primary impeller 6 and be driven radially out of the channels 34 by the vanes 10. In the illustrated embodiment, five (5) vanes 10 are used, but it should be appreciated that fewer than five or more than five vanes 10 can be used.

As shown in FIGS. 16A, 16C, and 17A, the secondary impeller 7 can have a proximal end 52 at a proximal edge of the vanes 10. Further as shown in FIGS. 15A and 15C, the drive unit 9 can have a distal end 53 at a distal end of the distally-projecting segments 33. As explained above, the distally projecting convex segments 33 can be received within the concave opening 32 of the rotor assembly 46. When the convex segments 33 are mated within the concave opening 32, the distal end of the drive unit 9 is distal the proximal end of the second impeller 7 (e.g., distal the proximal-most end of the rotor assembly).

FIGS. 15F and 15G illustrate additional examples of drive units 18A, 18B, according to various embodiments. Unless otherwise noted, components of FIGS. 15F-15G may be the same as or generally similar to like-numbered components of FIGS. 15A-15E. As above, the bearings 18A, 18B can include a plurality of distally-projecting segments 33 extending from a base 606 of the drive bearing 18A or 18B, the plurality of distally-projecting segments 33 spaced apart circumferentially to define at least one channel 34 between adjacent segments 33. As shown, the drive bearing 18A or 18B can comprise a curved and/or ramped surface 605 angled distally (and radially inwardly) from the base 606 and defining a portion of the at least one channel 34, e.g., the ramped surface 605 may converge inwardly. The ramped or curved surface 605, when considered in combination with the opposing features on the secondary impeller 7, can effectively create a converging or diverging channel 34 for blood egress. The converging or diverging channels 34 may function in multiple planes simultaneously if desired. Furthermore, the boundaries of the projections 33 can be varied to control the mean flow vector defining the exiting blood flow. The channel 34 can be defined as desired to vary the degree of flow vector and channeling desired. Further, the distally-extending projections 33 can extend distal the curved or ramped surface 605. Beneficially, the curved or ramped surface 605 can assist in guiding the flow of blood out of the secondary impeller 7.

Embodiments described herein are included to demonstrate particular aspects of the present disclosure. It should be appreciated by those of ordinary skill in the art that the embodiments described herein merely represent exemplary embodiments (e.g., non-limiting examples) of the disclosure. Those of ordinary skill in the art should, in light of the present disclosure, appreciate that many changes can be made in the specific embodiments described, including various combinations of the different elements, components, steps, features, or the like of the embodiments described, and still obtain a like or similar result without departing from the spirit and scope of the present disclosure. From the foregoing description, one of ordinary skill in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosure.

Prior work is detailed in U.S. Pat. No. 8,012,079 and U.S. Pat. Pub. No. 2017/0087288, which are both fully incorporated by reference herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example+5%, +10%, +15%, etc.). For example, “about 1” includes “1.” Phrases preceded by a term such as “substantially,” “generally,” and the like include the recited phrase and should be interpreted based on the circumstances (e.g., as much as reasonably possible under the circumstances). For example, “substantially spherical” includes “spherical.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y and at least one of Z to each be present.

Although certain embodiments and examples have been described herein, it should be emphasized that many variations and modifications may be made to the humeral head assembly shown and described in the present disclosure, the elements of which are to be understood as being differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, it will be understood by those skilled in the art that the scope of the inventions extends beyond the specifically disclosed embodiments to any and all embodiments having equivalent elements, modifications, omissions, combinations or sub-combinations of the specific features and aspects of the embodiments (e.g., of aspects across various embodiments), adaptations and/or alterations, and uses of the inventions as would be appreciated by those in the art based on the present disclosure. The limitations in the claims are to be interpreted fairly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

1. A blood flow assist system comprising:
a blood pump;
an elongate power lead having a distal end portion connected to the blood pump and a proximal end portion opposite the distal end portion, the power lead comprising:
a lumen extending distally from the proximal end portion of the power lead along a longitudinal axis of the blood flow assist system;
one or more electrical contacts disposed on an outer surface of the lead at the proximal end portion of the elongate power lead; and
a recess extending into the proximal portion of the power lead in a direction transverse to the longitudinal axis, the recess configured to receive a locking pin to releasably connect the power lead to an external device.

2. The blood flow assist system of claim 1, wherein the lumen is an inner lumen and further comprising a plurality of outer lumens disposed around the inner lumen, the plurality of outer lumens extending along the longitudinal axis.

3. The blood flow assist system of claim 2, further comprising a plurality of elongate conductors, each elongate conductor of the plurality of elongate conductors extending through a corresponding outer lumen of the plurality of outer lumens.

4. The blood flow assist system of claim 3, wherein the one or more electrical contacts comprises a plurality of electrical contacts spaced apart along the longitudinal axis on the outer surface of the lead, wherein each elongate conductor of the plurality of elongate conductors is electrically connected to a corresponding electrical contact of the plurality of electrical contacts.

5. The blood flow assist system of claim 4, wherein each electrical contact of the plurality of electrical contacts comprises a ring, wherein adjacent electrical contacts are spaced apart by an insulating material.

6. The blood flow assist system of claim 1, wherein the elongate lead comprises an insulating material along an outer surface thereof, the insulating material comprising polyurethane.

7. The blood flow assist system of claim 1, wherein the one or more electrical contacts are disposed distal the recess.

8. The blood flow assist system of claim 1, wherein the recess is disposed distal a proximal end of the elongate power lead.

9. The blood flow assist system of claim 1, wherein a thickness of the elongate power lead is not uniform along a length of the elongate power lead.

10. The blood flow assist system of claim 1 further comprising a delivery system to deliver the blood pump to a target location in a patient.

11. The blood flow assist system of claim 10, wherein the delivery system comprises:
a proximal handle having a lumen therethrough, the lumen sized and shaped to receive the proximal end portion of the elongate power lead, the proximal handle comprising a lead retention device connectable to the elongate power lead, the proximal handle further comprising a lead release assembly configured to release the elongate power lead from the lead retention device;
a delivery catheter extending distally from the proximal handle;
a transfer stop disposed about the delivery catheter distal the proximal handle, the transfer stop comprising a transfer stop lock having a locked configuration and an unlocked configuration, the delivery catheter slidable relative to the transfer stop in the unlocked configuration and slidably locked relative to the transfer stop in the locked configuration; and
a distal handle disposed about the delivery catheter distal the transfer stop, the distal handle comprising a handle lock having a locked configuration and an unlocked configuration, the delivery catheter slidable relative to the distal handle in the unlocked configuration and slidably locked relative to the distal handle in the locked configuration, the distal handle comprising a cavity configured to house the at least a distal portion of the blood pump.

12. The blood flow assist system of claim 1, further comprising a retrieval system configured to remove the blood pump from a patient.

13. The blood flow assist system of claim 12, wherein the retrieval system comprises:
a retrieval dilator having a retrieval dilator hub and a clamping member at a proximal portion of the retrieval dilator, the retrieval dilator having a lumen sized and shaped to receive the elongate power lead therethrough, the clamping member having a clamped configuration in which the clamping member clamps against the elongate power lead and an unclamped configuration in which the elongate power lead is slidable relative to the clamping member;
a retrieval sheath having a retrieval sheath hub at a proximal portion of the retrieval sheath, the retrieval sheath having a lumen sized and shaped to receive the retrieval dilator therethrough; and
a retrieval handle having a lumen sized and shaped to receive the retrieval dilator therethrough such that the retrieval dilator extends through the retrieval handle and the retrieval sheath during a retrieval procedure, the retrieval handle having a distal connector configured to connect to the retrieval sheath hub and a proximal connector configured to connect to the retrieval dilator hub.

14. A percutaneous medical system comprising:
a medical device;
an elongate lead having a distal end portion connected to the medical device and a proximal end portion opposite the distal end portion, the lead comprising:
a lumen extending distally from the proximal end portion of the lead along a longitudinal axis of the system; and
a recess extending into the proximal end portion of the lead in a direction transverse to the longitudinal axis, the recess configured to receive a locking pin to releasable connect the lead to an external device.

15. The percutaneous medical system of claim 14, wherein the elongate lead comprises an elongate power lead configured to convey current to the medical device.

16. The percutaneous medical system of claim 15, further comprising one or more electrical contacts disposed on an outer surface of the lead at the proximal end portion of the elongate power lead.

17. The percutaneous medical system of claim 14, further comprising a plurality of outer lumens disposed around the lumen, the plurality of outer lumens extending along the longitudinal axis.

18. The percutaneous medical system of claim 17, further comprising a plurality of elongate conductors, each elongate conductor of the plurality of elongate conductors extending through a corresponding outer lumen of the plurality of outer lumens.

19. The percutaneous medical system of claim 18, wherein each elongate conductor of the plurality of elongate conductors is electrically connected to a corresponding electrical contact on exposed on an outer surface of the lead.

20. The percutaneous medical system of claim 19, wherein each electrical contact comprises a ring, wherein adjacent electrical contacts are spaced apart by an insulating material.

21. The percutaneous medical system of claim 14, wherein the elongate lead comprises an insulating material along an outer surface thereof, the insulating material comprising polyurethane.

22. The percutaneous medical system of claim 14, wherein a thickness of the elongate lead is not uniform along a length of the elongate lead.

23-235. (canceled)
